(19) United States
(12) Patent Application Publication
(10) Pub. No.: US 2011/0281854 A1
(43) Pub. Date: Nov. 17, 2011

(54) MORPHOLINE COMPOUNDS

(75) Inventors: Agustin Casimiro-Garcia, Mystic, CT (US); Kentaro Futatsugi, Niantic, CT (US); David W. Piotrowski, Waterford, CT (US)

(73) Assignee: Pfizer Inc

(21) Appl. No.: 13/104,430

(22) Filed: May 10, 2011

Related U.S. Application Data
(60) Provisional application No. 61/333,511, filed on May 11, 2010.

(51) Int. Cl. A61K 31/5415; A61K 31/5383; C07D 413/04; A61K 31/5377; A61P 9/00; A61P 13/12; A61P 3/10; A61P 29/00; A61P 25/04; C07D 498/04; A61P 1/16

(52) U.S. Cl. 514/224.2; 544/105; 514/230.5; 544/131; 514/235.5; 544/52

(57) ABSTRACT

Mineralocorticoid receptor antagonists (MRa), pharmaceutical compositions containing such inhibitors and the use of such inhibitors to treat, for example, diabetic nephropathy and hypertension in mammals, including humans.

This application claims priority from U.S. Provisional Application 61/333,511 filed May 11, 2010.

### BACKGROUND OF THE INVENTION

This invention relates to compounds that are mineralocorticoid receptor antagonists (MRa), pharmaceutical compositions containing such antagonists and the use of such inhibitors to treat for example, diabetic nephropathy and hypertension.

Hypertension affects about 20% of the adult population in developed countries. In the adult population aged 60 years or older, this percentage increases to about 60% to 70%. Hypertension also is associated with an increased risk of other physiological complications including stroke, myocardial infarction, atrial fibrillation, heart failure, peripheral vascular disease and renal impairment. Although a number of anti-hypertensive drugs are available in various pharmacological categories, the efficacy and safety of such drugs can vary from patient to patient. There are a variety of physiological conditions associated with hypertension and one exemplary condition is diabetic nephropathy.

Mineralocorticoid receptor antagonists are one class of drugs that can be used to treat hypertension and/or related physiological complications (Jewell, C. W., et al., Cardiovascular & Hematological Agents in Medicinal Chemistry (2006) Vol. 4, pgs. 129-153). Mineralocorticoids, such as aldosterone, are involved in regulating salt and water balance in mammals. Activation of the mineralocorticoid receptor can induce hypertension and cause other detrimental cardiovascular and physiological effects. Two mineralocorticoid receptor antagonists, spironolactone (ALDACTONE™) and eplerenone (INSPRA™), are presently available and indicated for the treatment of hypertension and heart failure (Baxter, J. D., et al., Molecular and Cellular Endocrinology (2004) Vol. 217, pgs. 151-165).

WO 2008/053300 describes certain pyrazoline compounds as mineralocorticoid receptor antagonists.

WO 2006/015259 discloses bicyclic heterocyclic compounds including certain benzo[1,4]oxazin-3-one compounds that modulate the activity of steroid hormone nuclear receptors including the mineralocorticoid receptor (MR).

WO 2008/130616 discloses certain diaryl morpholines as CB1 modulators.

The present invention is particularly directed to mineralocorticoid receptor antagonists that are non-steroidal compounds. Use of a non-steroidal mineralocorticoid receptor antagonist potentially provides certain advantages over a steroidal mineralocorticoid receptor antagonist including, e.g., further improvement in selectivity with respect to the sex hormone receptors; less complex and costly chemical synthesis; and the like.

There remains a need for pharmaceutical agents that have MRa activity and are useful in the treatment, prevention or diminution of the manifestations of the maladies described herein.

### SUMMARY OF THE INVENTION

The present invention is directed to a compound of the Formula I,

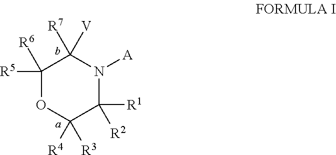

a prodrug thereof, or a pharmaceutically acceptable salt of said compound or of said prodrug;
wherein R¹ and R², are each independently H, (C₁-C₄)alkyl or cyclo(C₃-C₆)alkyl said (C₁-C₄)alkyl optionally mono-substituted with (C₁-C₄)alkoxy or cyano or optionally substituted with one to nine fluoros and said cyclo(C₃-C₆)alkyl optionally substituted with one to six fluoros;
wherein R³, R⁴, R⁵ and R⁶ are each independently H, phenyl, (C₁-C₄)alkyl, cyclo(C₃-C₆)alkyl, or cyclooxa(C₃-C₆)alkyl, said (C₁-C₄)alkyl optionally mono-substituted with (C₁-C₄)alkoxy or cyano or optionally substituted with one to nine fluoros and said cyclo(C₃-C₆)alkyl optionally substituted with one to six fluoros;
wherein at least three of R¹, R², R³, R⁴, R⁵ and R⁶ are H;
or wherein R³ and R⁴ can be linked together to form a three to six membered ring optionally having one oxygen, said ring optionally fused to phenyl;
wherein V is H, phenyl, naphthyl, (C₁-C₄)alkyl or cyclo(C₃-C₆)alkyl, said phenyl, naphthyl, (C₁-C₄)alkyl or cyclo(C₃-C₆)alkyl may optionally be mono-, di- or tri-substituted with R⁵ with the proviso that if V is H, then at least two of R¹, R², R³, R⁴, R⁵, R⁶ or R⁷ are not H;
R⁷ is H or wherein when V is phenyl V is optionally linked together with R⁷ to form a fused nine to ten membered carbobicyclic ring;
or wherein when V is phenyl it is optionally linked together with R⁵ to form the tricyclic moiety

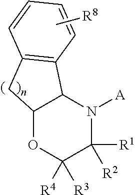

R⁸ is H, halo, (C₁-C₄)alkyl, cyclo(C₃-C₆)alkyl or (C₁-C₄)alkoxy, said (C₁-C₄)alkyl optionally substituted with from one to nine fluoros;
n is 1 or 2;
wherein A is

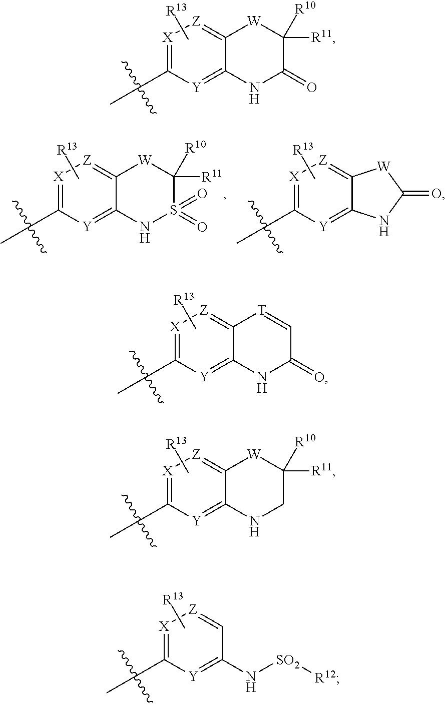

### T is CH or N;

X, Y and Z are independently CH or N;

### W is CH₂, O, S or NH;

R¹⁰ and R¹¹ are independently H or fluoro;
R¹² is (C₁-C₄)alkyl or cyclo(C₃-C₆)alkyl said (C₁-C₄)alkyl or cyclo(C₃-C₆)alkyl optionally substituted with one to nine fluoros; and
R¹³ is H, (C₁-C₄)alkyl, halo or cyano.

Yet another aspect of this invention is directed to a method for treating cardiovascular conditions, renal conditions, liver conditions, inflammatory conditions, pain, retinopathy, neuropathy, insulinopathy, diabetic nephropathy, edema, endothelial dysfunction or baroreceptor dysfunction in a mammal (including a human being either male or female) by administering to a mammal in need of such treatment a cardiovascular conditions, renal conditions, liver conditions, inflammatory conditions, pain, retinopathy, neuropathy, insulinopathy, diabetic nephropathy, edema, endothelial dysfunction or baroreceptor dysfunction treating amount of a compound of Formula I, a prodrug thereof, or a pharmaceutically acceptable salt of said compound or of said prodrug. A preferred method is wherein diabetic nephropathy is treated.

Also provided herein are compositions comprising a pharmaceutically effective amount of one or more of the compounds described herein and a pharmaceutically acceptable vehicle, carrier or excipient.

This invention is also directed to pharmaceutical combination compositions comprising: a therapeutically effective amount of a composition comprising

a first compound, said first compound being a Formula I compound, a prodrug thereof, or a pharmaceutically acceptable salt of said compound or of said prodrug;

a second compound, said second compound being an anti-hypertensive agent; and/or optionally

a pharmaceutical vehicle, diluent or carrier.

Preferably the second compound is a loop diuretic and it is especially preferred that it is torsemide.

All patents and patent applications referred to herein are hereby incorporated by reference.

Other features and advantages of this invention will be apparent from this specification and the appendant claims which describe the invention.

### BRIEF DESCRIPTION OF THE DRAWINGS

FIG. 1 is a characteristic x-ray powder diffraction pattern showing a crystalline form of Example 1, 6-((2R,5R)-2-methyl-5-phenylmorpholino)-2H-pyrido[3,2-b][1,4]oxazin-3(4H)-one (Vertical Axis: Intensity (CPS); Horizontal Axis: Two theta (degrees)).

FIG. 2 is an X-ray crystal structure (ORTEP drawing) of Example 1, 6-((2R,5R)-2-methyl-5-phenylmorpholino)-2H-pyrido[3,2-b][1,4]oxazin-3(4H)-one.

FIG. 3 is a characteristic X-ray powder diffraction pattern showing a crystalline form of Example 2, 2-((2R,5R)-2-methyl-5-phenylmorpholino)-6H-pyrimido[5,4-b][1,4]oxazin-7(8H)-one (Vertical Axis: Intensity (CPS); Horizontal Axis: Two theta (degrees)).

### DETAILED DESCRIPTION OF THE INVENTION

A preferred group of compounds, designated the A Group, contains those compounds having the Formula I as shown above wherein:

the morpholine C^{a} is (R);
the morpholine C^{b} is (R); and

### A is

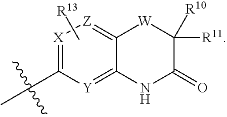

A group of compounds which is preferred among the A Group of compounds designated the B Group, contains those compounds wherein

V is phenyl;

### W is O;

### X is CH;

### Y is N;

### Z is CH;

R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

A group of compounds which is preferred among the B Group of compounds designated the C Group, contains those compounds wherein

R³ is (C₁-C₄)alkyl or cyclo(C₃-C₆)alkyl;
R⁸ is H, halo, (C₁-C₄)alkyl, cyclo(C₃-C₆)alkyl or (C₁-C₄)alkoxy;

### R¹⁰ and R¹¹ are H; and

### R¹³ is H.

A group of compounds which is preferred among the C Group of compounds designated the D Group, contains those compounds wherein

R³ is (C₁-C₄)alkyl; and
R⁸ is H, halo or (C₁-C₄)alkyl.

A preferred group of compounds, designated the E Group, contains those compounds having the Formula I as shown above wherein

the morpholine C^{a} is (R);
the morpholine C^{b} is (R);

### A is

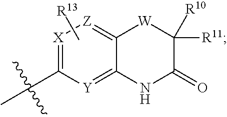

V is phenyl;

### W is O;

### X is CH;

### Y is CH;

### Z is CH;

R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

A preferred group of compounds, designated the F Group, contains those compounds having the Formula I as shown above wherein

the morpholine C^{a} is (R);
the morpholine C^{b} is (R);

### A is

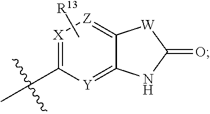

V is phenyl;

### W is O;

R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

A preferred group of compounds, designated the G Group, contains those compounds having the Formula I as shown above wherein

the morpholine C^{a} is (R);
the morpholine C^{b} is (R);

### A is

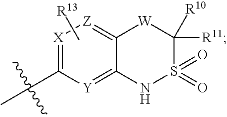

V is phenyl;

### W is O;

R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

A preferred group of compounds, designated the H Group, contains those compounds having the Formula I as shown above wherein

the morpholine C^{a} is (R);
the morpholine C^{b} is (R);

### A is

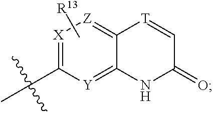

V is phenyl;
R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

A preferred group of compounds, designated the I Group, contains those compounds having the Formula I as shown above wherein

the morpholine C^{a} is (R);
the morpholine C^{b} is (R);

### A is

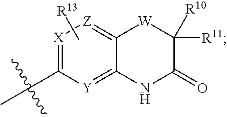

V is phenyl;

### W is O;

### X is N;

### Y is CH;

### Z is CH;

R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

A preferred group of compounds, designated the J Group, contains those compounds having the Formula I as shown above wherein

the morpholine C^{a} is (R);
the morpholine C^{b} is (R);

### A is

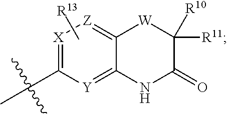

V is phenyl;

### W is O;

### X is N;

### Y is N;

### Z is CH;

R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

A preferred group of compounds, designated the K Group, contains those compounds having the Formula I as shown above wherein
6-((2R,5R)-2-methyl-5-phenylmorpholino)-2H-pyrido[3,2-b][1,4]oxazin-3(4H)-one,
6-((2R,5R)-5-(4-fluorophenyl)-2-methylmorpholino)-2H-pyrido[3,2-b][1,4]oxazin-3(4H)-one,
(R)-6-(2,2-dimethyl-5-phenylmorpholino)-2H-pyrido[3,2-b][1,4]oxazin-3(4H)-one,
2-((2R,5R)-2-methyl-5-phenylmorpholino)-6H-pyrimido[5,4-b][1,4]oxazin-7(8H)-one,
6-((2R,5R)-2-methyl-5-phenylmorpholino)-2H-benzo[b][1,4]oxazin-3(4H)-one,
7-((2R,5R)-2-methyl-5-phenylmorpholino)-1H-pyrido[3,4-b][1,4]oxazin-2(3H)-one,
2-((2R,5R)-4-(3-oxo-3,4-dihydro-2H-pyrido[3,2-b][1,4]oxazin-6-yl)-5-phenylmorpholin-2-yl)acetonitrile,
6-((2R,5R)-5-(2-fluorophenyl)-2-methylmorpholino)-2H-pyrido[3,2-b][1,4]oxazin-3(4H)-one,
6-(cis-2,6-dimethylmorpholino)-2H-pyrido[3,2-b][1,4]oxazin-3(4H)-one,
6-((2R,5R)-5-(3-fluorophenyl)-2-methylmorpholino)-2H-pyrido[3,2-b][1,4]oxazin-3(4H)-one,
2-((2R,5R)-5-(2-fluorophenyl)-2-methylmorpholino)-6H-pyrimido[5,4-b][1,4]oxazin-7(8H)-one,
7-((2R,5R)-5-(2-fluorophenyl)-2-methylmorpholino)-1H-pyrido[3,4-b][1,4]oxazin-2(3H)-one,
6-((2R,5R)-5-(2,4-difluorophenyl)-2-methylmorpholino)-2H-pyrido[3,2-b][1,4]oxazin-3(4H)-one,
6-((2S,5R)-2-(fluoromethyl)-5-phenylmorpholino)-2H-pyrido[3,2-b][1,4]oxazin-3(4H)-one,
6-((2S,3R,6R)-2,6-dimethyl-3-phenylmorpholino)-2H-pyrido[3,2-b][1,4]oxazin-3(4H)-one and
6-((2R,5R)-5-(2-chlorophenyl)-2-methylmorpholino)-2H-pyrido[3,2-b][1,4]oxazin-3(4H)-one.

An especially preferred compound is 6-(2-methyl-5-phenylmorpholino)-2H-pyrido[3,2-b][1,4]oxazin-3(4H)-one.

An especially preferred compound is 6-((2R,5R)-2-methyl-5-phenylmorpholino)-2H-pyrido[3,2-b][1,4]oxazin-3(4H)-one or a pharmaceutically acceptable salt thereof.

An especially preferred compound is the compound of Formula II

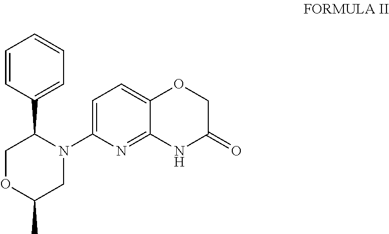

Pharmaceutically acceptable salts of the compounds of Formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts Properties, Selection, and Use by Stahl and Wermuth (Wiley-VCH, 2002).

The compounds of the invention may exist in both unsolvated and solvated forms. The term ‘solvate’ is used herein to describe a molecular complex comprising the compound of the invention and one or more pharmaceutically acceptable solvent molecules, for example, ethanol. Such solvent molecules are those commonly used in the pharmaceutical art, which are known to be innocuous to the recipient, e.g., water, ethanol, ethylene glycol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO. The term “hydrate” refers to the complex where the solvent molecule is water. The solvates and/or hydrates preferably exist in crystalline form.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the invention include compounds of Formula I as hereinbefore defined, polymorphs, and isomers thereof (including optical, geometric and tautomeric isomers) as hereinafter defined and isotopically-labelled compounds of Formula I.

The compounds of the present invention may be administered as prodrugs. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as ‘prodrugs’. [Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).]

Prodrugs can, for example, be produced by replacing appropriate functionalities present in the compounds of Formula I with certain moieties known to those skilled in the art as ‘pro-moieties’ as described, for example, in “Design of Prodrugs” by H Bundgaard (Elsevier, 1985).

Some examples of such prodrugs include:
(i) where the compound of Formula I contains a carboxylic acid functionality (—COOH), an ester thereof, for example, replacement of the hydrogen with (C₁-C₈)alkyl;
(ii) where the compound of Formula I contains an alcohol functionality (—OH), an ether thereof, for example, replacement of the hydrogen with (C₁-C₆)alkanoyloxymethyl; and
(iii) where the compound of Formula I contains a primary or secondary amino functionality (—NH₂ or —NHR where R≠H), an amide thereof, for example, replacement of one or both hydrogens with (C₁-C₁₀)alkanoyl.

In addition, certain compounds of Formula I may themselves act as prodrugs of other compounds of Formula I.

Compounds of Formula I containing an asymmetric carbon atom can exist as two or more stereoisomers. Where a compound of Formula I contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

The present invention includes all pharmaceutically acceptable isotopically-labelled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labelled compounds of Formula (I) can generally be prepared by conventional techniques known to those skilled in the art or by processes analogous to those described in the accompanying Examples and Preparations using an appropriate isotopically-labelled reagents in place of the non-labelled reagent previously employed.

References herein to “treatment” include curative, palliative and prophylactic treatment.

As used herein, the expressions “reaction-inert solvent” and “inert solvent” refer to a solvent or a mixture thereof which does not interact with starting materials, reagents, intermediates or products in a manner which adversely affects the yield of the desired product.

By “pharmaceutically acceptable” is meant the carrier, diluent, excipients, and/or salt must be compatible with the other ingredients of the Formulation, and not deleterious to the recipient thereof.

The term “pharmaceutically effective amount”, as used herein, refers to an amount of the compound of Formula I sufficient to treat, prevent onset of or delay or diminish the symptoms and physiological manifestations of the indications described herein.

The term “room temperature or ambient temperature” means a temperature between 18 to 25° C., “HPLC” refers to high pressure liquid chromatography, “MPLC” refers to medium pressure liquid chromatography, “TLC” refers to thin layer chromatography, “MS” refers to mass spectrum or mass spectroscopy or mass spectrometry, “NMR” refers to nuclear magnetic resonance spectroscopy, “DCM” refers to dichloromethane, “DMSO” refers to dimethyl sulfoxide, “DME” refers to dimethoxyethane, “EtOAc” refers to ethyl acetate, “MeOH” refers to methanol, “Ph” refers to the phenyl group, “Pr” refers to propyl, “trityl” refers to the triphenylmethyl group, “ACN” refers to acetonitrile, “DEAD” refers to diethylazodicarboxylate, and “DIAD” refers to diisopropylazodicarboxylate.

Alkyl, alkenyl and alkynyl groups and the alkyl portions of alkoxy groups discussed herein include straight or branched groups having the number of carbon atoms indicated including, for example, methyl, methoxy, ethyl, styrene, propyl, isopropyl, isopropyloxy, allyl, n-butyl, t-butyl, isobutyl, pentyl, isopentyl, and 2-methylbutyl groups. The terms halo or halogen refer to F, Cl, Br or I.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth. In general the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section.

Specific synthetic schemes for preparation of the compounds of Formula I are outlined below.

As an initial note, in the preparation of the Formula I compounds it is noted that some of the preparation methods useful for the preparation of the compounds described herein may require protection of remote functionality (e.g., primary amine, secondary amine, carboxyl in Formula I precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

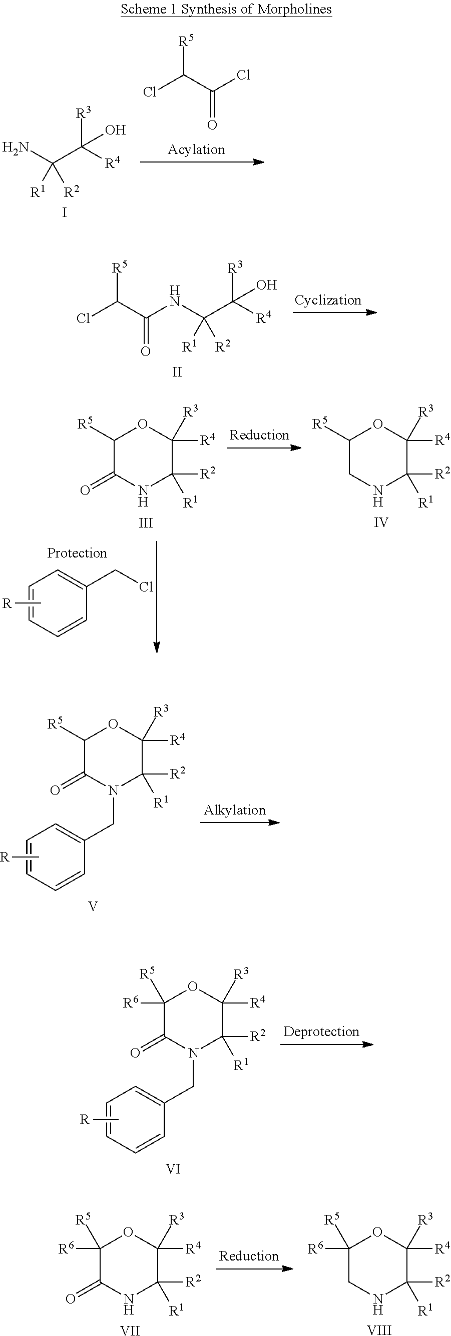

According to Scheme 1 the Formula VIII compounds wherein R¹, R², R³, R⁴, R⁵ and R⁶ are as defined above may be prepared from the Formula I compound by acylation, cyclization, protection, alkylation, deprotection and reduction.

For example, the Formula II compound may be conveniently prepared by combining the Formula I compound and a 2-halo acid chloride in an aprotic solvent such as dichloromethane or tetrahydrofuran in the presence of an organic base like triethylamine at a temperature of about 0° C. to about 60° C., typically less than 30° C., for about 30 minutes to about 24 hours.

Then the Formula II compound is treated with either potassium t-butoxide in a protic solvent such as t-butanol, or sodium hydride in an aprotic solvent such as tetrahydrofuran, at a temperature of about 20° C. to about 50° C., typically ambient, for about thirty minutes to about to about three hours to form the corresponding Formula III cyclic ether.

The Formula IV compound may be prepared by treating the Formula III cyclic ether with a reducing agent such as sodium bis(2-methoxyethoxy)aluminum hydride (Red-Al) or lithium aluminum hydride in an aprotic solvent such as toluene or tetrahydrofuran at a temperature of about −25° C. to about 25° C., typically about 5° C. for about twenty minutes to about two hours followed by stirring at ambient temperature for about six to about eighteen hours.

The Formula V protected amine may be prepared from the corresponding Formula III compound by treatment with an appropriate protecting agent. The Formula III compound, in an anhydrous solvent such as anhydrous DMF, is treated with a strong base such as sodium hydride at ambient temperature for about five minutes to about one hour. The resulting solution is combined with a benzyl halide at a temperature of about −25° C. to about 25° C., typically 0° C., followed by stirring at ambient temperature for about one to about eight hours.

The resulting Formula V compound is converted to the Formula VI compound by an alkylation reaction. The Formula V compound is treated with a strong non-nucleophilic base such as lithium diisopropylamide (LDA) in an anhydrous solvent such as tetrahydrofuran. Then the reaction is cooled to a temperature of about −100° C. to about −50° C. for about 10 minutes to about two hours. The resulting mixture is combined with the appropriate R⁶halide and allowed to warm to ambient over about two to about eighteen hours to achieve the desired Formula VI compound.

The Formula VI compound is deprotected using either hydrogenation or oxidizing conditions. The Formula VI compound is hydrogenated at elevated pressure, for example, a pressure of about 50 psi of hydrogen using a palladium catalyst such as 10% palladium on carbon in a protic solvent such as methanol in a Parr shaker at a temperature of about 10° C. to about 50° C., typically ambient, for about one hour to about eight hours to form the corresponding Formula VII substituted alpha oxo-morpholine. Alternatively, the Formula VI compound is treated with an oxidizing agent such as Ceric Ammonium Nitrate (CAN) in a solvent such as acetonitrile/water at a temperature of about 10° C. to about 50° C., typically ambient, for about one hour to about eight hours to form the corresponding Formula VII compound.

The Formula VII substituted morpholine compound can be prepared from the corresponding Formula V compound by reduction. For example, the Formula VII compound is treated with lithium aluminum hydride (LAH) in an anhydrous polar solvent such as tetrahydrofuran at a temperature of about 40° C. to about 70° C., typically reflux, for about one hour to about eight hours.

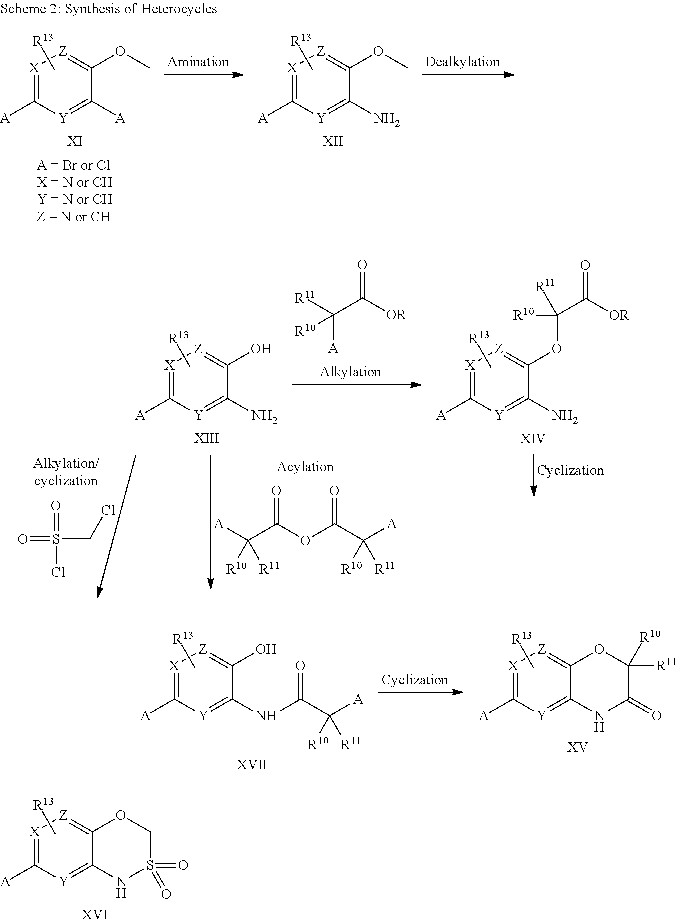

According to Scheme 2 the Formula XV compounds wherein R¹⁰, R¹¹, and R¹³ are as defined above may be prepared from the Formula XI compound by amination, deprotection, alkylation or acylation, and cyclization.

Thus, the Formula XII amine compounds wherein R¹³ is as defined above may be prepared from the corresponding Formula XI halo compound by reaction with ammonium hydroxide in an aprotic solvent such as dioxane at a temperature of about 80° C. to about 120° C., typically about 100° C., for about six hours to about twenty-four hours in a sealed reaction vessel.

The Formula XIII hydroxyl compound may be conveniently prepared from the corresponding Formula XII methoxy compound by dealkylation with an agent such as boron tribromide in a polar aprotic solvent such as methylene chloride at a temperature of about 15° C. to about 40° C., typically at ambient, for about two hours to about twelve hours.

Then the Formula XIII compound is combined with an alkyl haloacetate and a base such as potassium carbonate in an anhydrous solvent such as DMF at a temperature of about 15° C. to about 40° C., typically at ambient, for about two hours to about twelve hours to form the corresponding Formula XIV ether.

The Formula XV oxazinone compound may be conveniently prepared from the corresponding Formula XIV amine by cyclization with a base such as potassium carbonate in an anhydrous solvent such as DMF at a temperature of about 40° C. to about 80° C., typically about 60° C. for about two hours to about twelve hours.

In addition, according to Scheme 2 the Formula XIII compound may also be converted to the Formula XVI compound in two steps. First, alkylation with chloromethanesulfonyl chloride in the presence of a base such as pyridine in an anhydrous solvent such as tetrahydrofuran at a temperature of about 15° C. to about 40° C., typically at ambient, for about six hours to about twenty-four hours. This is followed by cyclization with a base such as potassium carbonate in a protic solvent such as methanol at a temperature of about 25° C. to about 80° C., typically about 60° C. for about two hours to about twelve hours to form the corresponding Formula XVI ether.

In addition, according to Scheme 2 the Formula XIII compound may also be converted to the Formula XVII compound by alkylation with a 2-halo substituted anhydride such as 2-chloro-2,2-difluoroacetic anhydride in the presence of a base such as triethylamine in an polar aprotic solvent such as dichloromethane at a temperature of about −15° C. to about 20° C., typically at 0° C., for about 10 minutes to about one hour followed by additional treatment at a temperature of about 15° C. to about 40° C., typically at ambient, for about one hour to about eight hours.

Then the Formula XVII compound is cyclized, in a protic solvent such as t-butanol, by treatment with a solution of a strong non-nucleophilic base such as potassium t-butoxide in a protic solvent such as t-butanol at a temperature of about 25° C. to about 100° C., typically ambient, for about three hours to about to about sixteen hours to form the corresponding Formula XV oxazinone.

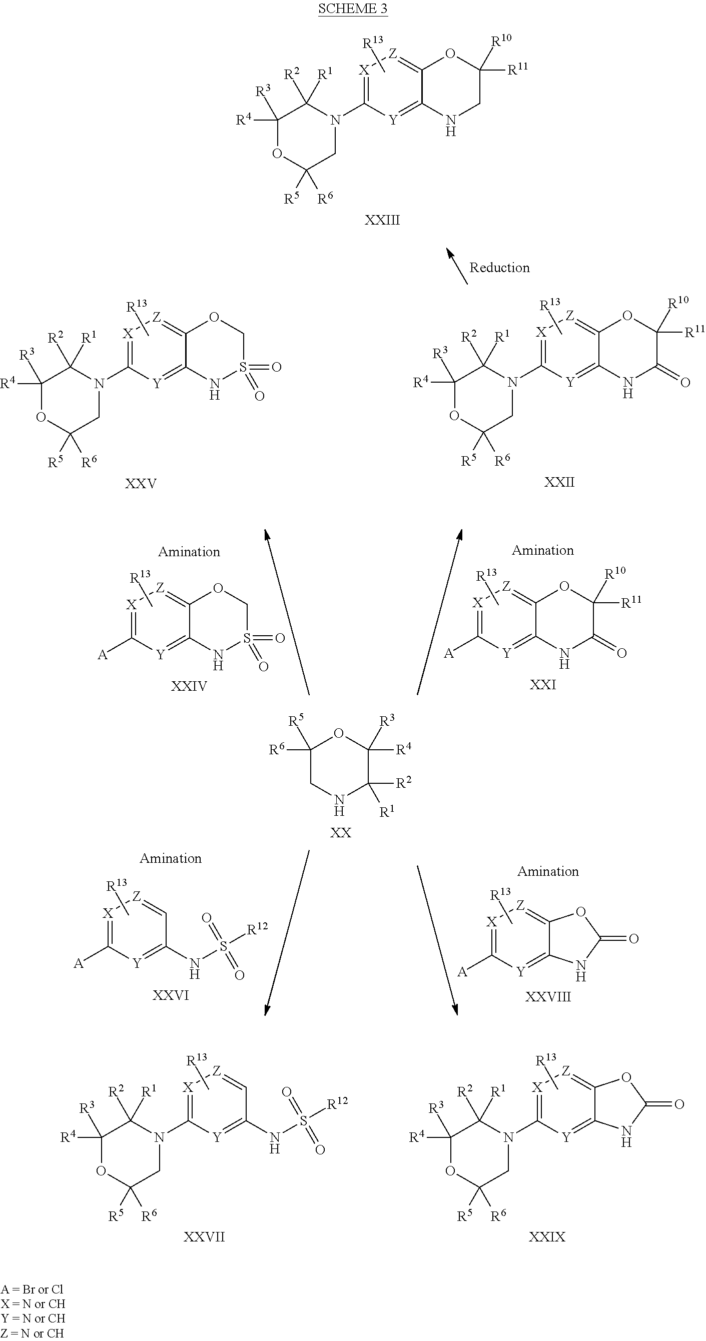

According to Scheme 3 the Formula XXII compounds wherein R¹, R², R³, R⁴, R⁵, R⁶, R¹⁰, R¹¹ and R¹³ are as defined above may be prepared from the Formula XXI compound by amination with a Formula XX compound.

The Formula XXII compound may be prepared by amination using the Buchwald-Hartwig cross coupling. Under these conditions, an organometallic catalyst such as tris(dibenzylideneacetone)dipalladium(0) (known as Pd₂(dba)₃) or Pd(OAc)₂ and a phosphine ligand such as 5-(diisopropylphosphino)-1′,3′,5′5-triphenyl-1′H-1,4′-bypyrazole (known as iPr-BiPPyPhos) are combined in a protic solvent such as t-amyl alcohol at a temperature of about 15° C. to about 40° C., typically ambient, for about 10 minutes to about two hours in a sealed container under nitrogen. The Formula XX compound, the Formula XXI compound and a polar aprotic solvent such as hexamethylphosphoramide (HMPA) or dimethylsulfoxide are added to the above mixture. Then a base such as solid lithium t-butoxide and/or a solution of lithium t-butoxide in a protic solvent such as t-amyl alcohol are added to the mixture at a temperature of about 25° C. to about 100° C., typically about 60° C. for about six hours to about 18 hours to form the corresponding Formula XXII compound.

By analogous means the Formula XXV, Formula XXVII and Formula XXIX compounds may be prepared by combining the Formula XX compound with the Formula XXIV, Formula XXVI and Formula XXVIII compounds respectively.

Alternatively, the Formula XXII compound may be prepared by a nucleophilic aromatic substitution by reacting the Formula XXI compound with the Formula XX amine in a polar aprotic solvent such as N-methylpyrrolidinone under microwave irradiation at a temperature of about 150° C. to about 225° C., typically about 100° C., for about 30 minutes to about three hours to form the corresponding Formula XXII compound.

The Formula XXIII compound can be conveniently prepared from the corresponding Formula XXII compound by reduction. For example, the Formula XXII compound is treated with lithium aluminum hydride (LAH) in an anhydrous aprotic solvent such as tetrahydrofuran at a temperature of about 40° C. to about 70° C., typically reflux for about one hour to about eight hours to form the corresponding Formula XXIII compound.

The starting materials and reagents for the above described Formula I compounds, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Cis/trans isomers may be separated by conventional techniques well known to those skilled in the art, for example, chromatography and fractional crystallization.

Mixtures of stereoisomers may be separated by conventional techniques known to those skilled in the art. [see, for example, “Stereochemistry of Organic Compounds” by E L Eliel (Wiley, New York, 1994).]

Conventional techniques for the preparation/isolation of individual enantiomers include chiral synthesis from a suitable optically pure precursor.

Alternatively, the racemate (or a racemic precursor) may be reacted with a suitable optically active compound, for example, an alcohol, or, in the case where the compound of Formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Pharmaceutically acceptable salts of compounds of Formula I may be prepared by one or more of three methods:
(i) by reacting the compound of Formula I with the desired acid or base;
(ii) by removing an acid- or base-labile protecting group from a suitable precursor of the compound of Formula I or by ring-opening a suitable cyclic precursor, for example, a lactone or lactam, using the desired acid or base; or
(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

The compounds of this invention may also be used in conjunction with other pharmaceutical agents (e.g., antihypertensive and antidiabetic agents) for the treatment of the disease/conditions described herein.

The compounds of the present invention may be used in combination with antihypertensive agents and such antihypertensive activity is readily determined by those skilled in the art according to standard assays (e.g., blood pressure measurements). Exemplary antihypertensive agents include renin inhibitors (e.g., aliskiren), aldosterone synthase inhibitors, calcium channel blockers, angiotensin converting enzyme inhibitors (ACE inhibitors), angiotensin II receptor antagonists (ARB antagonists), Beta-adrenergic receptor blockers (beta- or β-blockers), Alpha-adrenergic receptor blockers (alpha- or α-blockers), vasodilators such as cerebral vasodilators, coronary vasodilators, peripheral vasodilators and diuretics.

In one embodiment, one or more compounds of Formulae I or II may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™, ESIDRIX™ or HYDRODIURIL™), hydrochlorothiazide (such as MICROZIDE™ or ORETIC™), benzthiazide, hydroflumethiazide (such as SALURON™), bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™); (d) quinazoline-type diuretics such as quinethazone; and (e) potassium-sparing diuretics such as triamterene (such as DYRENIUM™), and amiloride (such as MIDAMOR™ or MODURETIC™).

In another embodiment, one or more compounds of Formulae I or II may be co-administered with a loop diuretic. In still another embodiment, the loop diuretic is selected from furosemide and torsemide. In still another embodiment, one or more compounds of Formulae I or II may be co-administered with furosemide. In still another embodiment, one or more compounds of Formulae I or II may be co-administered with torsemide which may optionally be a controlled or modified release form of torsemide.

In another embodiment, one or more compounds of Formulae I or II may be co-administered with a thiazide-type diuretic. In still another embodiment, the thiazide-type diuretic is selected from the group consisting of chlorothiazide and hydrochlorothiazide. In still another embodiment, one or more compounds of Formulae I or II may be co-administered with chlorothiazide. In still another embodiment, one or more compounds of Formulae I or II may be co-administered with hydrochlorothiazide.

In another embodiment, one or more compounds of Formulae I or II may be co-administered with a phthalimidine-type diuretic. In still another embodiment, the phthalimidine-type diuretic is chlorthalidone.

The compounds of the present invention may be used in combination with antidiabetic agents and such anti-diabetic activity is readily determined by those skilled in the art according to standard assays known in the art. Examples of such antidiabetic agents include an acetyl-CoA carboxylase-2 (ACC-2) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4, exenatide (Byetta™), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381(2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist, 11 Beta HSD and a glucokinase activator. Preferred anti-diabetic agents are metformin, glucagon-like peptide 1 (GLP-1) agonists (Byetta), and DPP-IV inhibitors (e.g., sitagliptin, vildagliptin, alogliptin and saxagliptin).

The compounds of the present invention may be used in combination with cholesterol modulating agents (including cholesterol lowering agents) such as a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, an ion-exchange resin, an antioxidant, an ACAT inhibitor or a bile acid sequestrant.

The compounds of the present invention can be used in combination with anti-obesity agents. Such anti-obesity activity is readily determined by those skilled in the art according to standard assays known in the art. Suitable anti-obesity agents include phenylpropanolamine, ephedrine, pseudoephedrine, phentermine, β₃ adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor (CB-1) antagonists (e.g., rimonabant described in U.S. Pat. No. 5,624,941 (SR-141,716A), purine compounds, such as those described in US Patent Publication No. 2004/0092520; pyrazolo[1,5-a][1,3,5]triazine compounds, such as those described in U.S. Non-Provisional patent application Ser. No. 10/763,105 filed on Jan. 21, 2004; and bicyclic pyrazolyl and imidazolyl compounds, such as those described in U.S. Provisional Application No. 60/518,280 filed on Nov. 7, 2003), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

The compounds of this invention may also be used in combination with a lipase inhibitor. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art.

In combination therapy treatment, both the compounds of this invention and the other drug therapies are administered to mammals (e.g., humans, male or female) by conventional methods.

The Formula I compounds of this invention, their prodrugs and the salts of such compounds and prodrugs are all adapted to therapeutic use as agents that mediate the mineralocorticoid receptor (MR) in mammals, particularly humans. For example, these compounds act as mineralocorticoid receptor antagonists (MRa) and thus are useful for the treatment of the various conditions (e.g., those described herein) in which such action is implicated.

It is believed that the mineralocorticoids, such as aldosterone, are involved in regulating salt and water balance in mammals. Activation of the mineralocorticoid receptor can induce hypertension and cause other detrimental cardiovascular and physiological effects. Accordingly, MR antagonists help to reduce hypertension and associated physiological effects.

Given the positive correlation between activation of the mineralocorticoid receptor with the development of cardiovascular and associated disease/conditions, Formula I compounds of this invention, their prodrugs and the salts of such compounds and prodrugs, by virtue of their pharmacologic action, are useful for the prevention, arrestment and/or regression of hypertension and its associated disease states. These include cardiovascular disorders (e.g., angina, cardiac ischemia and myocardial infarction) and other associated complications e.g., diabetic nephropathy.

The disease/conditions that can be treated in accordance with the present invention include, but are not limited to, cardiovascular conditions, renal conditions, liver conditions, vascular conditions, inflammatory conditions, pain, retinopathy, neuropathy (such as peripheral neuropathy), insulinopathy, edema, endothelial dysfunction, baroreceptor dysfunction and the like.

Cardiovascular conditions include, but are not limited to, hypertension, heart failure (such as congestive heart failure), diastolic dysfunction (such as left ventricular diastolic dysfunction, diastolic heart failure, and impaired diastolic filling), systolic dysfunction (such as systolic heart failure), arrhythmia, ischemia, hypertrophic cardiomyopathy, sudden cardiac death, myocardial and vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, fibrinoid necrosis of coronary arteries, stroke, and the like.

Renal conditions include, but are not limited to, glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, macroalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions affecting glomeruli and microvessels), and the like.

Liver conditions include, but are not limited to, liver cirrhosis, liver ascites, hepatic congestion, and the like.

Vascular conditions include, but are not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, and the like.

Inflammatory conditions include, but are not limited to, arthritis (for example, osteoarthritis), inflammatory airways diseases (for example, chronic obstructive pulmonary disease (COPD)), and the like.

Pain includes, but is not limited to, acute pain, chronic pain (for example, arthralgia), and the like.

Edema includes, but is not limited to, peripheral tissue edema, hepatic congestion, splenic congestion, liver ascites, respiratory or lung congestion, and the like.

Insulinopathies include, but are not limited to, insulin resistance, Type I diabetes mellitus, Type II diabetes mellitus, glucose sensitivity, pre-diabetic state, syndrome X, and the like.

In one embodiment, the condition is selected from the group consisting of cardiovascular conditions, renal conditions, and liver conditions.

In another embodiment, the condition is a cardiovascular condition.

In another embodiment, the condition is a cardiovascular condition selected from the group consisting of hypertension, heart failure (particularly heart failure post myocardial infarction), left ventricular hypertrophy, and stroke.

In another embodiment, the condition is hypertension.

In another embodiment, the condition is heart failure.

In another embodiment, the condition is left ventricular hypertrophy.

In another embodiment, the condition is stroke.

In another embodiment, the condition is a renal condition.

In another embodiment, the condition is diabetic nephropathy.

In another embodiment, the condition is Type II diabetes mellitus.

The compounds of Formula I can have improved solubility and selectivity across related nuclear hormone receptors including progesterone, androgen and glucocorticoid.

The utility of the Formula I compounds of the invention, their prodrugs and the salts of such compounds and prodrugs as medical agents in the treatment of the above described disease/conditions in mammals (e.g. humans, male or female) is demonstrated by the activity of the compounds of this invention in conventional in vitro and in vivo assays described below. The in vivo assays (with appropriate modifications within the skill in the art) may be used to determine the activity of other agents as well as the compounds of this invention. Such assays also provide a means whereby the activities of the Formula I compounds of this invention, their prodrugs and the salts of such compounds and prodrugs (or the other agents described herein) can be compared to each other and with the activities of other known compounds. The results of these comparisons are useful for determining dosage levels in mammals, including humans, for the treatment of such diseases.

The following protocols may of course be varied by those skilled in the art.

### Radioligand Binding Assay

To measure the affinity of test compound in the present invention for MR, and therefore have the capacity to modulate MR activity, radioligand displacement assays were performed. Test compound affinity was expressed as IC₅₀ value, defined as the concentration of test compound required to decrease [³H]aldosterone binding by 50%.

MR binding assays were performed in a final volume of 50 μL containing 1 nM of MR (GST-LBD fusion; expressed in SF9 insect cells), and 1 nM [³H]aldosterone (PerkinElmer, NET419) plus varying concentrations of test compound or vehicle.

Briefly, assays were prepared at 4° C. in 384-well plate (Costar, 3657) containing 1 μl of test compound in DMSO (or DMSO as vehicle). Assays were initiated by addition of 24 μL of 2 nM [³H]aldosterone followed by 25 μL of 2 nM GST-MR in binding-wash buffer (50 mM HEPES (pH 7.5), 50 mM KCl, 2 mM EDTA, 10% glycerol and 5 mM DTT).

The mixture was incubated at 4° C. for 4 hrs, then was transferred to a 384-well glass fiber filtration plate (Millipore, MZFCN0W50) previously treated with 0.5% PEI. The mixture was suctioned dry with vacuum and immediately washed three times with 100 μL of 4° C. binding-wash buffer. The plates were allowed to air dry overnight at room temperature, 7 μL of Ready Safe Liquid Scintillant (Beckman, 141349) was added to each well, and the amount of receptor-ligand complex was determined by liquid scintillation counting using a 1450 Microbeta Trilux (Wallac).

Radioligand binding filtration format assays for progesterone receptor (PR) and glucocorticoid receptor (GR) were performed essentially as described for MR. Full length PR (Invitrogen, P2835) or GR-LBD (Invitrogen, PV4690) were used at 8 nM final concentration. [³H]progesterone (Perkin Elmer, NET381) or [³H]dexamethasone (PerkinElmer, NET467), 5 nM final concentration, were substituted for radiolabeled aldosterone.

### Cell-Based Reporter Assay

To measure the ability of test compound in the present invention to modulate the activity of MR (agonize, antagonize, partially agonize, partially antagonize), bioassays were performed that measured the modulation of reporter gene expression. Cells were transiently transfected with a luciferase reporter gene under the control of a Gal4 response element (Gal4-RE-luc) and a plasmid containing the Gal4 DNA binding domain fused to the MR ligand binding domain (Gal4-MR-LBD). Agonists can bind to and activate the MR-LBD, which activates the expression of the luciferase reporter gene through interaction with the Gal4 response element. Cells were treated with a submaximal level of ligand (˜EC₈₀) in the presence or absence of compounds. Antagonists can compete for binding to the NHR-LBD and decrease the agonist-induced transcriptional activity of the reporter gene. Therefore, measurement of luciferase activity allowed quantitative determinations of the reporter transcription in the presence of either agonists or competitive antagonists.

Briefly, human liver cells (Huh7, ATCC) were transfected using FuGENE™ 6 Transfection Reagent according to the manufacturer's instructions (Roche Molecular Biochemicals, 11814443001). Approximately 24 hours after transfection, the cells were harvested in phenol red-free RPMI1640 media containing 10% charcoal-and-dextran stripped serum (HyClone, SH30068.03), and plated in 45 μl at 10,000 cells per well in white tissue culture 384-microplates (Greiner bio-one 781080). Test compounds were prepared at 200-fold final concentrations in 100% DMSO and diluted 20-fold in assay buffer containing aldosterone at ten-times EC₈₀ (concentration required for 80% of full activation for MR). To test for receptor antagonism, cells were incubated for approximately 3 hours and then treated with 5 μL of the test compound aldosterone mixture at final EC₈₀ (concentration required for 80% of full activation for MR) plus test compound. The final concentration of DMSO in the test plate was 0.5%. Following an overnight incubation with compound, 25 μL of Steady-Glow™ lysis buffer with luciferase substrate (Promega Corporation, E2550) was added directly to the cells. After a 30-minute incubation to completely lyse the cells, the microplates were counted in an Envision™ Multilabel Reader (Perkin Elmer) in single photon counting mode. In antagonist mode, compound efficacy was expressed as IC₅₀ value, defined as the concentration of test compound required to decrease the EC₈₀ aldosterone signal by 50%.

| Example | MR IC₅₀ (μM) |
|---|---|
| 1 | 0.0444 |
| 2 | 0.266 |
| 3 | 0.157 |
| 4 | 0.306 |
| 5 | 0.174 |
| 6 | 0.977 |
| 7 | 0.0636 |
| 8 | 0.0334 |
| 9 | 0.137 |
| 10 | 0.105 |
| 11 | 0.0899 |
| 12 | 0.151 |
| 13 | 0.407 |
| 14 | 1.1 |
| 15 | 1.22 |
| 16 | 1.97 |
| 17 | 3.05 |
| 18 | 2.71 |
| 19 | 5.61 |
| 20 | 3 |
| 21 | 0.583 |
| 22 | 0.885 |
| 23 | 0.284 |
| 24 | 1.77 |
| 25 | 0.798 |
| 26 | 3.37 |
| 27 | 1.23 |
| 28 | 0.024 |
| 29 | 0.055 |
| 30 | 9.56 |
| 31 | 0.913 |
| 32 | 0.111 |
| 33 | 0.0539 |
| 34 | 0.146 |
| 35 | 0.104 |
| 36 | 1.74 |
| 37 | 1.44 |
| 38 | 4.97 |
| 39 | 0.0867 |
| 40 | 0.0979 |
| 41 | 5.23 |
| 42 | 0.527 |
| 43 | 0.395 |
| 44 | 1.23 |
| 45 | 0.349 |
| 46 | 0.252 |
| 47 | 1.63 |
| 48 | 1.38 |
| 49 | 0.358 |
| 50 | 0.272 |
| 51 | 0.537 |
| 52 | 0.278 |
| 53 | 7.45 |
| 54 | 2.8 |
| 55 | 0.485 |
| 56 | 5.33 |
| 57 | 4.69 |
| 58 | 2.99 |
| 59 | 0.703 |
| 60 | 9.56 |
| 61 | 0.646 |
| 62 | 7.91 |
| 63 | 4.27 |
| 64 | 0.449 |
| 65 | 0.631 |
| 66 | 0.451 |
| 67 | 0.135 |
| 68 | 2.28 |
| 69 | 0.204 |
| 70 | 0.628 |
| 71 | 1.93 |
| 72 | 8.75 |
| 73 | 6.34 |
| 74 | 3.42 |
| 75 | 3.08 |

Cell-based reporter assays measuring the ability of test compound to modulate the activity of PR and GR were performed in an identical manner as described for MR except that cells were transfected with plasmid encoding the appropriate Gal4-HNR-LBDs. Progesterone (50 nM) and dexamethasone (100 nM) were used as agonists, respectively. Androgen receptor assays were performed by transfecting AR Gal4-LBD in a 96-well format (Corning, 3596) using 30,000 cells/well in a volume of 100 μL. Test compound and dihydrotestosterone (10 nM) were added in a 3-fold concentrated stock in 50 μL volume and Steady-Glow™ lysis buffer was added in 50 μL volume.

### Cell-Based Phenotypic Assay

To measure the ability of test compound in the present invention to antagonize the activity of PR, bioassays were performed that measured the functional effects on endogenousely expressed PR in T47D mammary carcinoma cells. In this system, PR activation induces alkaline phosphatase (AP) expression and this effect can be inhibited by antagonists.

Briefly, T47D cells (ATCC, HTB-133) were plated at 15,000 cells/well in 45 μL assay media consisting of phenol free RPMI (Gibco, 11835), 10% charcoal-stripped FBS (Hyclone SH30068-03), 2 mM Glutamine, 10 mM HEPES, and 1 mM sodium pyruvate in white tissue culture 384-microplates (Greiner bio-one 781080)). Test compounds were prepared at 200-fold final concentrations in 100% DMSO and diluted 20-fold in assay buffer containing progesterone at ten-times EC₈₀ (concentration required for 80% of full activation for PR. To test for receptor antagonism, cells were incubated for approximately 3 hours and then treated with 5 μL of the test compound progesterone mixture. The final concentration of DMSO in the test plate was 0.5%. After an overnight incubation, cells were washed in PBS and lysed by freeze thaw. Alkaline phosphatase activity was quantitated after addition of 10 μL/well TROPIX CSPD Ready-to-use Emerald II reagent (Applied Biosystems, T2212), according to the manufacturers instructions. Compound efficacy was expressed as IC₅₀ value, defined as the concentration of test compound required to decrease the response of 5 nM progesterone by 50%.

### Assessment of Urinary Na⁺/K⁺ Excretion

To determine the effect of MR antagonism on electrolyte balance, urinary Na⁺/K⁺ excretion was quantified in rats. All procedures were conducted in accordance with Institutional Animal Care and Use Committee guidelines and regulations at Pfizer Inc. (Groton, Conn.).

Female Wistar rats (400 g) were obtained from Charles River, Wilmington Mass. Rats were housed on a 12 hour light/dark cycle, and were provided food and water ad libitum. Prior to the onset of the study, rats were acclimated to metabolism cages for urine collection. On the day of the study, rats (n=7/group) were dosed by oral gavage with either vehicle (2% polyvinyl pyrrolidone/0.025% sodium lauryl sulfate) or test compound in a total volume of 5 mL/Kg. Following dosing, urine was collected from 0 hr (when the dose was administered) to 2 hrs, from 2 hrs to 4 hrs, from 4 hrs to 6 hrs and from 6 hrs to 8 hrs. Urine volume was measured and samples were assayed for Na⁺ and K⁺ measurement using a Siemens Advia 1800 chemistry analyzer and the Log 10*(Na⁺/K⁺) was calculated.

Administration of the compounds of this invention can be via any method which delivers a compound of this invention systemically and/or locally. These methods include oral routes, parenteral, intraduodenal routes, buccal, intranasal etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

For administration to human patients, an oral daily dose of the compounds herein may be in the range 1 mg to 500 mg depending, of course, on the mode of and frequency of administration, the disease state, and the age and condition of the patient, etc. An oral daily dose is in the range of 3 mg to 250 mg may be used. A further oral daily dose is in the range of 5 mg to 180 mg. The total daily dose may be administered in single or divided doses and may, at the physician's discretion, fall outside of the typical ranges given herein.

For convenience, the compounds of the present invention can be administered in a unit dosage form. If desired, multiple doses per day of the unit dosage form can be used to increase the total daily dose. The unit dosage form, for example, may be a tablet or capsule containing about 0.01, 0.05, 0.1, 0.5, 1, 5, 10, 15, 20, 25, 30, 35, 40, 45, 50, 55, 60, 65, 70, 75, 80, 85, 90, 95, 100, 125, 150, 175, 200, 250 or 500 mg of the compound of the present invention. In one embodiment, the unit dosage form contains from about 0.01 mg to about 500 mg of the compound of the present invention. In another embodiment, the unit dosage form contains from about 0.05 mg to about 250 mg of the compound of the present invention. In another embodiment, the unit dosage form contains from about 0.1 mg to about 200 mg of the compound of the present invention. In another embodiment, the unit dosage form contains from about 0.5 mg to about 150 mg of the compound of the present invention.

These compounds may also be administered to animals other than humans, for example, for the indications detailed above. The precise dosage administered of each active ingredient will vary depending upon any number of factors, including but not limited to, the type of animal and type of disease state being treated, the age of the animal, and the route(s) of administration.

A dosage of the combination pharmaceutical agents to be used in conjuction with the Formula I compounds is used that is effective for the indication being treated. Such dosages can be determined by standard assays such as those referenced above and provided herein. The combination agents may be administered simultaneously or sequentially in any order.

These dosages are based on an average human subject having a weight of about 60 kg to 70 kg. The physician will readily be able to determine doses for subjects whose weight falls outside this range, such as infants and the elderly.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

The present invention further comprises use of a compound of Formulae I or II for use as a medicament (such as a unit dosage tablet or unit dosage capsule). In another embodiment, the present invention comprises the use of a compound of Formulae I or II for the manufacture of a medicament (such as a unit dosage tablet or unit dosage capsule) to treat one or more of the conditions previously identified in the above sections discussing methods of treatment. In one embodiment, the condition is hypertension. In another embodiment the condition is diabetic nephropathy.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The compounds described herein may be administered as a formulation comprising a pharmaceutically effective amount of a compound of Formula I, in association with one or more pharmaceutically acceptable excipients. The term “carrier” or “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solid dosage form such a tablet, capsule, or a solution or suspension suitable for oral, parenteral, intradermal, subcutaneous, or topical application. Excipients can include, by way of illustration and not limitation, diluents, disintegrants, binding agents, adhesives, wetting agents, polymers, lubricants, glidants, stabilizers, substances added to mask or counteract a disagreeable taste or odor, flavors, dyes, fragrances, and substances added to improve appearance of the composition. Acceptable excipients include (but are not limited to) stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutically acceptable materials. Examples of excipients and their use may be found in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

The compounds herein may be formulated for oral, buccal, intranasal, parenteral (e.g., intravenous, intramuscular or subcutaneous) or rectal administration or in a form suitable for administration by inhalation. The compounds of the invention may also be formulated for sustained delivery.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, 20th Edition (Lippincott Williams & Wilkins, 2000).

Pharmaceutical compositions according to the invention may contain 0.1%-95% of the compound(s) of this invention, preferably 1%-70%. In any event, the composition or Formulation to be administered will contain a quantity of a compound(s) according to the invention in an amount effective to treat the disease/condition of the subject being treated, e.g., (hypertension, diabetic nephropathy).

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Also, as the present invention has an aspect that relates to the treatment of the disease/conditions described herein with a combination of active ingredients which may be administered jointly, the invention also relates to combining separate pharmaceutical compositions in a single dosage form, such as (but not limited to) a single tablet or capsule, a bilayer or multilayer tablet or capsule, or through the use of segregated components or compartments within a tablet or capsule.

The compounds of this invention either alone or in combination with each other or other compounds generally will be administered in a convenient formulation. The following formulation examples only are illustrative and are not intended to limit the scope of the present invention.

In the formulations which follow, “active ingredient” means a compound of this invention.

The active ingredient may be delivered as a suspension or nanosuspension in an aqueous vehicle such as 0.5% methylcellulose in water or 2% polyvinyl pyrrolidone/0.025% sodium lauryl sulfate in water.

The active ingredient may be delivered as a solution in an aqueous or non-aqueous vehicle, with or without additional solvents, co-solvents, excipients, or complexation agents selected from pharmaceutically acceptable diluents, excipients, vehicles, or carriers.

The active ingredient may be formulated as a solid dispersion or as a self emulsified drug delivery system (SEDDS) with pharmaceutically acceptable excipients.

The active ingredient may be formulated as an immediate release or modified release tablet or capsule. Alternatively, the active ingredient may be delivered as the active ingredient alone within a capsule shell, without additional excipients.

### General Experimental Procedures

All chemicals, reagents and solvents were purchased from commercial sources when available and used without further purification. Proton nuclear magnetic spectroscopy (¹H NMR) was recorded with 400 and 500 MHz Varian spectrometers. Chemical shifts are expressed in parts per million downfield from tetramethylsilane. The peak shapes are denoted as follows: s, singlet; d, doublet; t, triplet; q, quartet; m, multiplet; br s, broad singlet. Mass spectrometry (MS) was performed via atmospheric pressure chemical ionization (APCI) or electron scatter (ES) ionization sources. Silica gel chromatography was performed primarily using a medium pressure Biotage or ISCO systems using columns pre-packaged by various commercial vendors including Biotage and ISCO. Microanalyses were performed by Quantitative Technologies Inc. and were within 0.4% of the calculated values. The terms “concentrated” and “evaporated” refer to the removal of solvent at reduced pressure on a rotary evaporator with a bath temperature less than 60° C. The abbreviation “min” and “h” stand for “minutes” and “hours” respectively.

The X-ray powder diffraction pattern was generated using a Bruker D5005 diffractometer equipped with a Cu radiation source, fixed slits (divergence 1.0 mm, anti-scatter 0.6 mm, and receiving 0.6 mm) and a Sol-X detector. Data was collected in the Theta-2 Theta goniometer configuration from a samples prepared on “0”-background quartz sample holders at the Cu wavelength Kα₁=1.54056 and Kα₂=1.54439 (relative intensity 0.5) from 4.0 to 40.0 degrees 2-Theta using a step size of 0.040 degrees and a step time of 1.0 second. X-ray tube voltage and amperage were set at 40 kV and 40 mA respectively. Data were collected and analyzed using Bruker DIFFRAC Plus software. Experiments were conducted at room temperature conditions.

### Preparative HPLC Method A:

### Column: Waters Sunfire C₁₈ 19×100 mm, 5 μm

Mobile Phase A: 0.05% trifluoroacetic acid in water (v/v)
Mobile phase B: 0.05% trifluoroacetic acid in acetonitrile (v/v)
Gradient: as specified in Example
Flow rate: 25.0 mL/min

### Preparative HPLC Method B:

### Column: Waters Sunfire C₁₈ 19×100 mm, 5 μm

Mobile Phase A: 0.05% formic acid in water (v/v)
Mobile phase B: 0.05% formic acid in acetonitrile (v/v)
Gradient: as specified in Example
Flow rate: 25.0 mL/min

### Preparative HPLC Method C:

### Column: Waters XBridge C₁₈ 19×100 mm, 5 μm

Mobile Phase A: 0.03% ammonium hydroxide in water (v/v)
Mobile phase B: 0.05% ammonium hydroxide in acetonitrile (v/v)
Gradient: as specified in Example
Flow rate: 25.0 mL/min

### Preparative HPLC Method D:

### Column: Phenomenex Gemini C₁₈ 250×21.2 mm, 5 μm

Solvent: acetonitrile/ammonium hydroxide (pH10)
Gradient: as specified in Example
Flow rate: 25.0 mL/min

### Analytical LCMS Method A:

### Column: Waters Atlantis C₁₈ 4.6×50 mm, 5 μm

Gradient: 95% water/acetonitrile linear gradient to 5% water/acetonitrile over 4 min; hold at 5% water/95% acetonitrile to 5.0 min.
Modifier: 0.05% trifluoroacetic acid
Flow rate: 2.0 mL/min

### Analytical LCMS Method B:

### Column: Waters XBridge C₁₈ 4.6×50 mm, 5 μm

Gradient: 95% water/acetonitrile linear gradient to 5% water/acetonitrile over 4 min; hold at 5% water/95% acetonitrile to 5.0 min.
Modifier: 0.03% ammonium hydroxide
Flow rate: 2.0 mL/min

### Analytical LCMS Method C:

### Column: Welch XB C₁₈ 2.1×50 mm, 5 μm

Gradient: Gradient: 1% A linear gradient to 100% B over 4 min.
Mobile Phase A: 0.0375% trifluoroacetic acid in water
Mobile Phase B: 0.01875% trifluoroacetic acid in acetonitrile
Flow rate: 0.8 mL/min

### EXAMPLES

### Preparation 1

### (±)-cis-2-methyl-5-phenylmorpholine

To a 0° C. solution of (±)-cis-2-methyl-5-phenylmorpholin-3-one (U.S. Pat. No. 7,629,338, 433 mg, 2.26 mmol) in tetrahydrofuran (15 mL) was added lithium aluminum hydride (2 M solution in THF, 2.26 mL, 4.53 mmol). The reaction mixture was stirred at reflux for 4 h. The reaction was quenched with water (40 mL) and extracted with dichloromethane (3×100 mL). The organic layers were combined, dried over magnesium sulfate, filtered and concentrated under vacuum. The residue was filtered through silica gel, eluting with 10% methanol in dichloromethane. The filtrate was concentrated to provide the title compound (288 mg, 63%) as an oil. ¹H NMR (400 MHz, CHLOROFORM-d) δ ppm 1.37 (2H, m), 1.42 (3H, m), 2.86 (1H, m), 3.04 (1H, dt, J=12.0, 3.0 Hz), 3.96 (3H, m), 4.14 (1H, m), 7.38 (1H, m), 7.45 (2H, m), 7.60 (1H, m)

### Preparation 2

### (2R,5R)-2-methyl-5-phenylmorpholine

### Step 1: (2R,5R)-2-methyl-5-phenylmorpholin-3-one

A solution of 2-chloro-N—((R)-2-hydroxy-1-phenylethyl)propanamide (U.S. Pat. No. 7,629,338, 60 g, 260 mmol) in t-butanol (540 mL) was added to a stirred suspension of potassium t-butoxide (59.1 g, 527 mmol) in t-butanol (920 mL) at room temperature. The reaction mixture was stirred for 1 h. The pH of the reaction mixture was adjusted to 4 by adding aqueous hydrogen chloride (1N, 140 mL). The mixture was concentrated to remove the t-butanol. Ethyl acetate (1000 mL) and water (500 mL) were added. After the layers were separated, the organic layer was washed with saturated aqueous sodium chloride (250 mL), dried over sodium sulfate, filtered and concentrated to provide a solid. The solid was completely dissolved in hot heptanes/ethyl acetate. The product precipitated upon cooling to room temperature overnight. The solid was filtered and dried to yield the title compound (33.75 g, 67%). ¹H NMR (400 MHz, CHLOROFORM-d) δ ppm 1.54 (3H, d, J=7.0 Hz), 3.84 (1H, ddd, J=11.9, 4.5, 0.8 Hz), 4.00 (1H, dd, J=11.9, 4.1 Hz), 4.34 (1H, q, J=7.0 Hz), 4.62 (1H, m), 7.34 (5H, m)

### Step 2: (2R,5R)-2-methyl-5-phenylmorpholine

A solution of (2R,5R)-2-methyl-5-phenylmorpholin-3-one (32 g, 167.3 mmol) in toluene (600 mL) was added to an ice cooled solution of sodium bis(2-methoxyethoxy) aluminum hydride (65% wt in toluene, 300 mL, 1000 mmol). The reaction mixture was stirred at 5° C. for 1 h and stirred at room temperature overnight. Aqueous sodium hydroxide (2 M, 700 mL, 1390 mmol) was added to the reaction mixture, allowing the temperature to rise to 45° C. The solution was diluted with toluene (100 mL) and the layers were separated. The organic layer was washed with aqueous potassium carbonate (10%, 100 mL), dried over sodium sulfate, filtered, and concentrated to afford the title compound (31.0 g, 100%) as an oil. ¹H NMR (400 MHz, DMSO-d₆) δ ppm 1.13 (3H, d, J=6.4 Hz), 2.52 (1H, dd, J=12.2, 5.6 Hz), 2.61 (1H, br s), 2.75 (1H, m), 3.59 (1H, m), 3.70 (2H, m), 3.81 (1H, m), 7.18 (1H, m), 7.29 (2H, m), 7.45 (2H, m).

### Preparation 3

### (2S,5R)-2-methyl-5-phenylmorpholine

### Step 1: (R)-4-(4-methoxybenzyl)-5-phenylmorpholin-3-one

To a 0° C. solution of (R)-5-phenylmorpholin-3-one (U.S. Pat. No. 7,629,338, 1 g, 5.64 mmol) in anhydrous N,N-dimethylformamide (5 mL) was added sodium hydride (60% dispersion in oil, 239 mg, 5.98 mmol). The mixture was stirred at room temperature for 15 min and then cooled to 0° C. before p-methoxybenzyl chloride (0.830 mL, 5.98 mmol) was added. The reaction mixture was stirred at room temperature for 4 h, diluted with ethyl acetate and washed with water. The aqueous layer was extracted with ethyl acetate. The combined organic layers were washed with saturated aqueous sodium chloride, dried over magnesium sulfate, filtered, and concentrated. The crude residue was purified by silica gel column chromatography (gradient: 20-50% ethyl acetate/heptanes) to provide the title compound (1.4 g, 83%) as a white solid. ¹H NMR (400 MHz, DMSO-d₆) δ ppm 3.33 (1H, d, J=14.8 Hz), 3.70 (3H, s), 3.73 (1H, m), 3.93 (1H, dd, J=11.9, 3.7 Hz), 4.23 (2H, m), 4.38 (1H, m), 5.16 (1H, d, J=14.8 Hz), 6.85 (2H, m), 7.04 (2H, m), 7.25 (2H, m), 7.34 (3H, m)

### Step 2: (2S,5R)-4-(4-methoxybenzyl)-2-methyl-5-phenylmorpholin-3-one

To a solution of diisopropylamine (1.1 mL, 7.7 mmol) in tetrahydrofuran (10 mL) at −78° C. was added N-butyllithium (2.5 M in hexanes, 3 mL, 7.7 mmol). The solution was stirred at 0° C. for 15 min and then cooled to −78° C. A solution of (R)-4-(4-methoxybenzyl)-5-phenylmorpholin-3-one (1.84 g, 6.2 mmol) in tetrahydrofuran (10 mL) was added. After stirring at −78° C. for 30 min, methyl iodide (0.56 mL, 8.67 mmol) was added. The reaction mixture was warmed up to room temperature overnight. The reaction mixture was poured into aqueous hydrochloric acid (1 N) and the mixture was extracted with ethyl acetate 3 times. The combined organic layers were dried over sodium sulfate, filtered and concentrated. The residue was purified by silica gel column chromatography (gradient: 0-60% ethyl acetate in heptanes) to provide the title compound (1.69 g, 87%) containing 15% of the cis diastereoisomer. ¹H NMR (400 MHz, CHLOROFORM-d) δ ppm 1.59 (d, J=7.4 Hz, 3H), 3.39 (d, J=14.4 Hz, 1H), 3.67 (dd, J=12.2, 7.9 Hz, 1H), 3.81 (s, 3H), 4.04 (dd, J=12.2, 4.6 Hz, 1H), 4.41-4.48 (m, 2H), 5.44 (d, J=14.4 Hz, 1H), 6.80-6.84 (m, 2H), 6.96-7.02 (m, 2H), 7.17-7.22 (m, 2H), 7.35-7.44 (m, 3H)

### Step 3: (2S,5R)-2-methyl-5-phenylmorpholin-3-one

To a solution of (2S,5R)-4-(4-methoxybenzyl)-2-methyl-5-phenylmorpholin-3-one (1.69 g, 1.57 mmol) in 50% acetonitrile/water (48 mL) was added ammonium cerium (IV) nitrate (6.04 g, 10.9 mmol). The reaction mixture was stirred at room temperature for 4 h, poured into aqueous hydrochloric acid (1 N) and extracted with ethyl acetate (2×100 mL). The combined organic layers were dried over sodium sulfate, filtered and concentrated. The residue was purified by silica gel column chromatography (eluent: 50% ethyl acetate/heptanes) to provide the title compound (502 mg, 48.4%) as a solid. ¹H NMR (400 MHz, CHLOROFORM-d) δ ppm 1.54 (d, J=6.8 Hz, 3H), 3.53 (dd, J=11.9, 10.0 Hz, 1H), 4.03-4.09 (m, 1H), 4.24-4.31 (m, 1H), 4.82 (dd, J=10.0, 4.5 Hz, 1H), 6.04 (br s, 1H), 7.30-7.44 (m, 5H)

### Step 4: (2S,5R)-2-methyl-5-phenylmorpholine

The title compound was prepared from (2S,5R)-2-methyl-5-phenylmorpholin-3-one by the general method used for Preparation 1 to give the title compound (382 mg, 82%) as an oil. ¹H NMR (400 MHz, CHLOROFORM-d) δ ppm 1.20 (d, J=6.3 Hz, 3H), 2.75 (dd, J=11.5, 10.2 Hz, 1H), 3.06 (dd, J=11.5, 2.3 Hz, 1H), 3.43-3.52 (m, 1H), 3.67-3.73 (m, 1H), 3.73-3.78 (m, 1H), 3.84-3.92 (m, 2H), 7.25-7.42 (m, 5H).

### Preparation 4

### (2R,5R)-5-(4-fluorophenyl)-2-methylmorpholine

The title compound was prepared by the method described in Preparation 2, Step 1 and Preparation 1 to give the title compound (54.6 g, 90%) as a yellow oil. ¹H NMR (400 MHz, CHLOROFORM-d) δ ppm 1.29 (d, 3H), 2.72 (dd, 1H), 2.90 (dd, 1H), 3.88-3.77 (m, 3H), 4.00 (dd, 1H), 7.01 (dt, 2H), 7.46 (dt, 2H)

### Preparation 5

### (±)-trans-5-(4-fluorophenyl)-2-methylmorpholine

The title compound was prepared by the general method used for Preparation 3 and Preparation 1, Step 2. ¹H NMR (400 MHz, CHLOROFORM-d) δ ppm 1.20 (3H, d, J=6.44 Hz), 2.74 (1H, dd, J=11.71, 10.15 Hz), 3.05 (1H, dd, J=11.71, 2.34 Hz), 3.42 (1 H, dd, J=10.83, 10.05 Hz), 3.62-3.74 (1H, m), 3.77-3.90 (2H, m), 6.97-7.06 (2H, m), 7.32-7.40 (2H, m)

### Preparation 6

### (±)-cis-5-(2-fluorophenyl)-2-methylmorpholine

The title compound was prepared from 2-amino-2-(2-fluorophenyl)ethanol by the general method used for Preparation 2. ¹H NMR (400 MHz, CHLOROFORM-d) δ ppm 1.24 (d, 3H), 1.77 (brs, 1H), 2.64 (dd, 1H), 2.82 (dd, 1H), 3.84 (m, 1H), 3.96 (dd, 1H), 4.10 (dd, 1H), 4.20 (t, 1H), 7.04 (m, 1H), 7.12 (dt, 1H), 7.24 (m, 1H), 7.78 (dt, 1H)

### Preparation 7

### (±)-cis-5-(3-fluorophenyl)-2-methylmorpholine

The title compound was prepared from 2-amino-2-(3-fluorophenyl)ethanol by the general method used for Preparation 2. ¹H NMR (400 MHz, CHLOROFORM-d) δ ppm 1.28 (d, 3H), 2.71 (dd, 1H), 2.89 (dd, 1H), 3.89-3.79 (m, 3H), 4.02 (dd, 1H), 6.99-6.92 (m, 1H), 7.33-7.22 (m, 3H).

### Preparation 8

### (R)-2,2-dimethyl-5-phenylmorpholine

The title compound was prepared from (2R,5R)-2-methyl-5-phenylmorpholin-3-one (Preparation 2, Step 1) by the general method used for Preparation 3. ¹H NMR (400 MHz, CHLOROFORM-d) δ ppm 1.21 (3H, s), 1.43 (3H, s), 2.86 (2H, m), 3.62 (2 H, m), 3.83 (1H, m), 7.27 (1H, m), 7.32 (2H, m), 7.40 (2H, m).

### Preparation 9

### (2S,5R)-2-(methoxymethyl)-5-phenylmorpholine

### Step 1: (2S,5R)-4-benzyl-2-(methoxymethyl)-5-phenylmorpholine

To a solution of ((2S,5R)-4-benzyl-5-phenylmorpholin-2-yl)methanol (European Journal of Organic Chemistry (2007), (13), 2100; 100 mg, 0.353 mmol) in N,N-dimethylformamide (2 mL) at 0° C. was added sodium hydride (17 mg, 60% dispersion in oil, 0.424 mmol). The solution was stirred at 0° C. for 30 min. Methyl iodide (0.068 mL, 1.06 mmol) was added. The solution was stirred overnight at room temperature. To the reaction mixture was added ethyl acetate. The mixture was extracted with saturated aqueous ammonium chloride and saturated aqueous sodium chloride. The organic layer was dried over sodium sulfate, filtered, concentrated and purified by column chromatography to afford the title compound (62 mg, 59%). ¹H NMR (400 MHz, CHLOROFORM-d) δ ppm 2.39 (1H, dd, J=12.1, 3.7 Hz), 2.73 (1H, dd, J=12.1, 3.1 Hz), 2.98 (1H, d, J=13.7 Hz), 3.39 (3H, s), 3.49 (2H, m), 3.71 (2H, m), 3.82 (1H, d, J=8.6 Hz), 3.99 (2H, m), 7.22 (2H, m), 7.25 (2H, s), 7.32 (4H, m), 7.47 (2H, m).

### Step 2: (2S,5R)-2-(methoxymethyl)-5-phenylmorpholine

A mixture of (2S,5R)-4-benzyl-2-(methoxymethyl)-5-phenylmorpholine (350 mg, 1.18 mmol), methanol (10 mL), p-toluenesulphonic acid (452 mg, 2.35 mmol) and 10% palladium on carbon (50% water wet, 251 mg, 0.118 mmol) was hydrogenated in a Parr shaker for 1 hour at 50 psi hydrogen. The mixture was filtered through Celite and concentrated. The residue was dissolved in dichloromethane and extracted with 4.3% aqueous sodium hydrogen carbonate. The layers were separated and the organic layer was washed with saturated aqueous sodium chloride, dried over sodium sulfate, filtered, and concentrated to provide the title compound (193 mg, 79%) as a light yellow solid. ¹H NMR (400 MHz, DMSO-d₆) δ ppm 2.71 (2H, dd, J=12.2, 4.4 Hz), 2.80 (1H, m), 3.24 (3 H, s), 3.49 (1H, m), 3.58 (2H, m), 3.71 (3H, m), 7.22 (1H, m), 7.29 (2H, m), 7.42 (2 H, m).

### Preparation 10

### (2R,5R)-5-(2,4-difluorophenyl)-2-methylmorpholine

The title compound was prepared by the general method used for Preparation 2, Step 1 and Preparation 1. ¹H NMR (400 MHz, CHLOROFORM-d) δ ppm 7.77 (1H, m), 6.82 (2H, m), 4.15 (1H, m), 4.08 (1H, dd), 3.95 (1H, dd), 3.82 (1H, m), 2.81 (1H, dd), 2.62 (1H, m), 1.23 (3H, d).

### Preparation 11

### (±)-cis-5-(2-methoxyphenyl)-2-methylmorpholine

The title compound was prepared by the general method used for Preparation 2, Step 1 and Preparation 1. ¹H NMR (400 MHz, CHLOROFORM-d) δ ppm 7.71 (1H, dd), 7.25 (1H, dt), 6.95 (1H, t), 6.87 (1H, d), 4.16 (2H, m), 4.00 (1H, dd), 3.83 (4H, m), 2.75 (1H, dd), 2.58 (1H, dd), 2.21 (1H, brs), 1.22 (3H, d).

### Preparation 12

### (4aR,9aS)-2-methyl-2,3,4,4a,9,9a-hexahydroindeno[2,1-b][1,4]oxazine

The title compound was prepared from (1R,2S)-1-amino-2,3-dihydro-1H-inden-2-ol and 2-chloropropionyl chloride by the general method used for Preparation 2, Step 1 and Preparation 1 to give the title compound (3.23 g, 73%) as an oil. ¹H NMR (400 MHz, CHLOROFORM-d) δ ppm 7.33 (2H, m), 7.21 (6H, m), 4.44 (1H, q), 4.32 (1H, t), 4.16 (1H, d), 4.04 (1H, d), 3.78 (1H, m), 3.52 (1H, m), 3.28 (1H, q), 3.00-2.81 (4H, m), 2.62-2.39 (2H, m), 2.16 (2H, s), 1.19 (3H, d), 0.96 (3H, d).

### Preparation 13

### (±)-cis-5-(4-fluorophenyl)-2-methylmorpholine

The title compound was prepared from 2-amino-2-(4-fluorophenyl)ethanol by the method described in Preparation 1 to give the title compound (367 mg, 76.7%) as an oil. ¹H NMR (400 MHz, CHLOROFORM-d) δ ppm 1.31 (d, J=6.4 Hz, 3H), 2.72 (dd, J=12.0, 6.0 Hz, 1H), 2.92 (dd, J=12.0, 3.2 Hz, 1H), 3.78-3.83 (m, 1H), 3.83-3.89 (m, 2H), 4.01 (dd, J=11.3, 5.3 Hz, 1H), 7.03 (t, J=8.8 Hz, 2H), 7.45-7.52 (m, 2H)

### Preparation 14

### (2R,5R)-2-cyclopropyl-5-phenylmorpholine

The title compound was prepared from 2-bromo-2-cyclopropylacetyl chloride (WO 2008027284) and (R)-2-amino-2-phenylethanol by the general method from Preparation 2, Step 1 and Preparation 1. ¹H NMR (400 MHz, CHLOROFORM-d) δ ppm 0.20 (1H, m), 0.40 (1H, m), 0.57 (2H, m), 1.46 (1H, m), 1.74 (1H, br s), 2.83 (1H, m), 3.00 (2H, m), 3.76 (1H, dd, J=11.4, 3.7 Hz), 3.88 (1H, m), 4.06 (1H, dd, J=11.4, 6.4 Hz), 7.25-7.36 (3H, m), 7.48-7.51 (2H, m).

### Preparation 15

### 2,3-dihydrospiro[indene-1,3′-morpholine]

The title compound was prepared from (1-amino-2,3-dihydro-1H-inden-1-yl)methanol and 2-chloroacetyl chloride using general method from Preparation 2, Step 1 and Preparation 1. ¹H NMR (400 MHz, CHLOROFORM-d) δ ppm 1.61 (1H, br s), 1.83-1.93 (1H, m), 2.68-2.77 (1H, m), 2.81-2.92 (2H, m), 2.93-3.02 (1H, m), 3.12-3.20 (1H, m), 3.48-3.54 (2H, m), 3.70-3.78 (1H, m), 3.83-3.89 (1H, m), 7.17-7.24 (3H, m), 7.41-7.46 (1H, m)

### Preparation 16

### (2S,5R)-2-(fluoromethyl)-5-phenylmorpholine

### Step 1: ((2S,5R)-5-phenylmorpholin-2-yl)methanol

The title compound was prepared by the general method used for Preparation 9, Step 2. ¹H NMR (400 MHz, METHANOL-d₄) δ ppm 2.85 (2H, d, J=5.3 Hz), 3.67 (2H, m), 3.82 (3H, m), 4.00 (1H, m), 4.83 (2H, s), 7.23 (1H, m), 7.32 (2H, m), 7.47 (2H, m).

### Step 2: (2S,5R)-2,2,2-trichloroethyl 2-(hydroxymethyl)-5-phenylmorpholine-4-carboxylate

2,2,2-trichloroethyl chloroformate (0.63 g, 0.41 mL) in tetrahydrofuran (5 mL) was added to a mixture of ((2S,5R)-5-phenylmorpholin-2-yl)methanol (700 mg, 1.92 mmol), tetrahydrofuran (50 mL) and aqueous sodium hydroxide (1 M, 10 mL, 10 mmol). The mixture was stirred at room temperature overnight and concentrated. The residue was extracted with ethyl acetate (2×50 mL). The organic layers were combined, extracted with aqueous hydrochloric acid (1 M) and saturated aqueous sodium chloride, dried over magnesium sulfate, filtered, and concentrated. The material obtained was purified by silica gel column chromatography (gradient: 0 to 100% ethyl acetate in heptanes) to afford the title compound (220 mg, 53.6%). ¹H NMR (400 MHz, CHLOROFORM-d) δ ppm 2.04 (1H, m), 3.01 (1H, m), 3.55 (1H, m), 3.69 (2H, m), 3.94 (2H, m), 4.47 (1H, dd, J=19.3, 12.1 Hz), 4.80 (2H, m), 5.21 (1H, m), 7.33 (3H, m), 7.48 (2H, d, J=7.6 Hz).

### Step 3: (2S,5R)-2,2,2-trichloroethyl 2-(fluoromethyl)-5-phenylmorpholine-4-carboxylate-trichloromethyl 2-(fluoromethyl)-5-phenylmorpholine-4-carboxylate

A mixture of ((2S,5R)-2,2,2-trichloroethyl 2-(hydroxymethyl)-5-phenylmorpholine-4-carboxylate (200 mg, 0.564 mmol) and dichloroethane (2 mL) was cooled to 0° C. and bis-(2-methoxyethyl)aminosulfur trifluoride (50% solution in toluene, 0.62 mL, 1.7 mmol) was added. The reaction solution was stirred at 0° C. for 2 h and at room temperature overnight. The mixture was then partitioned between dichloromethane (50 mL) and an aqueous sodium hydroxide (1 N, 20 mL). The organic layer was extracted with saturated aqueous sodium chloride, dried over sodium sulfate, filtered and concentrated. The residue was purified by silica gel column chromatography (gradient: 0 to 100% ethyl acetate in heptanes to afford the title compound (96 mg, 46%). ¹H NMR (400 MHz, CHLOROFORM-d) δ ppm 3.05 (1H, m), 3.82 (1H, m), 4.01 (2H, m), 4.39 (1 H, d, J=4.3 Hz), 4.48 (2H, m), 4.83 (2H, m), 5.24 (1H, m), 7.33 (3H, m), 7.53 (2H, m).

### Step 4: (2S,5R)-2-(fluoromethyl)-5-phenylmorpholine

A mixture of (2S,5R)-2,2,2-trichloroethyl 2-(fluoromethyl)-5-phenylmorpholine-4-carboxylate-trichloromethyl 2-(fluoromethyl)-5-phenylmorpholine-4-carboxylate (90 mg, 0.24 mmol), acetic acid (2 mL) and zinc powder (640 mg, 4.9 mmol) was stirred at 60° C. overnight and concentrated. The residue was diluted in methanol (10 mL) and filtered through celite. The filtrate was concentrated and dissolved in methanol (1 mL) and loaded onto a Waters Oasis MCX SPE cartridge and eluted with methanol and ammonia in methanol (2 M) to afford the title compound (30 mg, 27%). ¹H NMR (400 MHz, CHLOROFORM-d) δ ppm 2.01 (2H, m), 3.03 (2H, m), 3.86 (1H, m), 3.99 (2H, m), 4.70 (1H, m), 7.30 (3H, m), 7.48 (2H, m)

### Preparation 17

### (±)-cis-5-(2-chlorophenyl)-2-methylmorpholine

The title compound was prepared from 2-amino-2-(2-chlorophenyl)ethanol by the general method used for Preparation 2, Step 1 and Preparation 1. 1H NMR (400 MHz, CHLOROFORM-d) δ ppm 1.22 (3H, d), 2.00 (1H, s), 2.58 (1H, d) 2.77 (1H, dd), 3.84 (1H, m), 4.05 (2H, m), 4.23 (1H, q), 7.25 (2H, m), 7.35 (1H, d), 7.92 (1H, d)

### Preparation 18

### (2S,3R,6R)-2,6-dimethyl-3-phenylmorpholine

The title compound was prepared from (1R,2S)-1-amino-1-phenylpropan-2-ol (Tetrahedron: Asymmetry 2006, 17 (3), 372) by the general method used for Preparation 2. ¹H NMR (400 MHz, DMSO-d₆) δ ppm 0.87 (3H, d, J=6.6 Hz), 1.09 (3H, d, J=6.2 Hz), 2.44 (2H, m), 3.51 (1H, m), 3.62 (1H, m), 3.94 (1H, m), 7.23 (4H, m), 7.47 (1H, m).

### Preparation 19

### (R)-7-phenyl-2,5-dioxa-8-azaspiro[3.5]nonane

### Step 1: (R)-(4-benzyl-5-phenylmorpholine-2,2-diyl)dimethanol

To a 0° C. mixture of ((2S,5R)-4-benzyl-5-phenylmorpholin-2-yl)methanol (European Journal of Organic Chemistry (2007), (13), 2100; 14.2 g, 50.2 mmol), dichloromethane (120 mL), triethylamine (35 mL, 0.25 mol) and dimethylsulfoxide (53 mL, 0.75 mol) was added sulfur trioxide pyridine complex (12.0 g, 75.2 mmol) in 4 portions. The mixture was stirred at 0° C. for 1 h and at room temperature for 16 h. Additional sulfur trioxide pyridine complex (4.0 g, 25.1 mmol) was added and stirring continued for 2 h. Water was added to the reaction and the layers were separated. The aqueous layer was extracted with dichloromethane. The combined organics were extracted with water (2×) and saturated aqueous sodium chloride, dried over magnesium sulfate, filtered and concentrated to provide (5R)-4-benzyl-5-phenylmorpholine-2-carbaldehyde as an oil. To a solution of (5R)-4-benzyl-5-phenylmorpholine-2-carbaldehyde (14.10 g, 50.2 mmol) in ethanol (350 mL) was added paraformaldehyde (30.1 g, 1.00 mol) at room temperature. The mixture was heated to 50° C. and a solution of sodium ethoxide in ethanol (21%, 33 mL, 0.10 mmol) was added. The reaction mixture was stirred at 50° C. overnight and cooled to room temperature. Saturated aqueous ammonium chloride was cautiously added followed by ethyl acetate and water. The layers were separated. The aqueous phase was extracted with ethyl acetate. The combined organics were extracted with saturated aqueous ammonium chloride, water and saturated aqueous sodium chloride. The organic layer was dried over magnesium sulfate, filtered and concentrated. The residue obtained was purified by silica gel column chromatography (eluent: 50% ethyl acetate in heptane) to provide the title compound (5.78 g, 37% over 2 steps) as an orange oil. ¹H NMR (300 MHz, CHLOROFORM-d) δ ppm 2.33 (1H, d), 2.79 (2H, m), 3.49 (3H, m), 3.80 (3H, m), 4.09 (2H, m), 7.35 (10H, m).

### Step 2: (R)-8-benzyl-7-phenyl-2,5-dioxa-8-azaspiro[3.5]nonane

To a −5° C. solution of (R)-(4-benzyl-5-phenylmorpholine-2,2-diyl)dimethanol (4.87 g, 15.6 mmol) in tetrahydrofuran (97 mL) was added n-butyllithium (2.01 M in hexanes, 7.7 mL, 15.0 mmol). The reaction was stirred at 0° C. for 30 min. A solution of p-toluenesulfonyl chloride (2.99 g, 15.7 mmol) in tetrahydrofuran (30 mL) was added, maintaining the temperature below 5° C. The reaction was stirred at room temperature for 1.25 h then quenched by the addition of saturated aqueous ammonium chloride. Ethyl acetate and saturated aqueous ammonium chloride were added and the layers separated. The aqueous phase as extracted with ethyl acetate. The combined organics were extracted with saturated aqueous sodium chloride, dried under magnesium sulfate, filtered and concentrated to afford ((5R)-4-benzyl-2-(hydroxynnethyl)-5-phenylmorpholin-2-yl)methyl 4-methylbenzenesulfonate (7.58 g) as an oil. To a −5° C. solution of ((5R)-4-benzyl-2-(hydroxymethyl)-5-phenylmorpholin-2-yl)methyl 4-methylbenzenesulfonate (7.58 g, 16.2 mmol) in tetrahydrofuran (97 mL) was added n-butyl lithium (2.01 M in hexanes, 12.0 mL, 15.0 mmol). The reaction was warmed up to room temperature and then heated to 60° C. for 18 h. Additional n-butyl lithium (4.00 mL, 8.00 mmol) was added three more times. The reaction was cooled to room temperature and quenched the addition of saturated aqueous ammonium chloride. The mixture was extracted with ethyl acetate (3×). The combined organic layer was extracted with water and saturated aqueous sodium chloride, dried under magnesium sulfate, filtered and concentrated. The residue was purified by silica gel column chromatography (gradient: 15-20% ethyl acetate in heptanes) to afford the tile compound (2.91 g, 63% over 2 steps) as a white solid. ¹H NMR (400 MHz, CHLOROFORM-d) δ ppm 2.21 (1H, d), 2.89 (1H, d), 3.25 (1H, d), 3.47-3.35 (2H, m), 3.69 (1H, dd), 3.83 (1H, d), 4.26 (1H, d), 4.52 (1H, d), 4.59 (1H, d), 4.74 (1H, dd), 7.46-7.22 (10H, m).

### Step 3: (R)-7-phenyl-2,5-dioxa-8-azaspiro[3.5]nonane

The title compound was prepared from (R)-8-benzyl-7-phenyl-2,5-dioxa-8-azaspiro[3.5]nonane by the general method used for Preparation 9, Step 2. ¹H NMR (300 MHz, CHLOROFORM-d) δ ppm 3.05 (1H, d), 3.41-3.29 (1H, m), 3.50 (1H, d), 3.72 (1H, dd), 3.87 (1H, dd), 4.37 (1H, d), 4.56 (1H, d), 4.74 (2H, s), 7.42-7.23 (5 H, m).

### Preparation 20

### 3′,4′-dihydro-2′H-spiro[morpholine-2,1′-naphthalene]

The title compound may be prepared from 1-(aminomethyl)-1,2,3,4-tetrahydronaphthalen-1-ol and 2-chloroacetyl chloride using general method from Preparation 2, Step 1 and Preparation 1. ¹H NMR (400 MHz, DMSO-d₆) δ ppm 1.69 (2H, m), 1.78 (1H, m), 2.72 (3H, m), 3.01 (1H, m), 3.13 (2H, m), 3.34 (1H, d, J=13.1 Hz), 3.80 (1H, m), 3.97 (1H, m), 7.07 (1H, m), 7.18 (2H, m), 7.58 (1H, m).

### Preparation 21

### Spiro[chroman-4,2′-morpholine]

The title compound may be prepared from 4-(aminomethyl)chroman-4-ol and 2-chloroacetyl chloride using general method from Preparation 2, Step 1 and Preparation 1. ¹H NMR (400 MHz, DMSO-d₆) δ ppm 2.13 (1H, m), 2.79 (1H, m), 3.13 (2H, m), 3.19 (1H, m), 3.50 (1H, d, J=13.1 Hz), 3.83 (1H, m), 3.94 (1H, m), 4.16 (2H, m), 6.75 (1H, dd, J=8.2, 1.4 Hz), 6.90 (1H, m), 7.18 (1H, m), 7.52 (1H, dd, J=7.9, 1.7 Hz), 9.70 (1H, m).

### Preparation 22

### 2-chloro-6H-pyrimido[5,4-b][1,4]oxazin-7(8H)-one and 2-bromo-6H-pyrimido[5,4-b][1,4]oxazin-7(8H)-one

### Step 1: 4-amino-2-chloropyrimidin-5-ol and 4-amino-2-bromopyrimidin-5-ol

A mixture of 2-chloro-5-methoxypyrimidin-4-amine (WO 2007/077961; 10.0 g, 62.5 mmol), dichloromethane (600 mL) and boron tribromide (20 mL) was stirred at room temperature overnight. Methanol was added until the solution was homogenous. The solution was concentrated to give a mixture of the title compounds (8.0 g, 89%) as a yellow solid, which was used for the next step without further purification. ¹H NMR (400 MHz, DMSO-d₆): δ ppm 5.21 (s, 3H), 7.50 (s, 1H).

### Step 2: ethyl 2-(4-amino-2-chloropyrimidin-5-yloxy)acetate and ethyl 2-(4-amino-2-bromopyrimidin-5-yloxy)acetate

A mixture of 4-amino-2-chloropyrimidin-5-ol and 4-amino-2-bromopyrimidin-5-ol (3.5 g, 24 mmol), N,N-dimethylformamide (50 mL), potassium carbonate (1.66 g, 12 mmol) and ethyl bromoacetate (4.0 g, 24 mmol) was stirred at room temperature overnight. The mixture was diluted with water (50 mL) and extracted with ethyl acetate (5×100 mL). The organic layers were combined, extracted with water (3×30 mL) and aqueous saturated sodium chloride, dried over sodium sulfate and concentrated. The residue was solidified from petroleum ether/ethyl acetate to give a mixture of the title compounds (3.0 g, 55%) as a solid. ¹H NMR (400 MHz, DMSO-d₆): δ ppm 1.21 (t, 3H), 4.21-4.14 (m, 2H), 4.83 (s, 2H), 7.6 (s, 1H).

### Step 3: 2-chloro-6H-pyrimido[5,4-b][1,4]oxazin-7(8H)-one and 2-bromo-6H-pyrimido[5,4-b][1,4]oxazin-7(8H)-one

A mixture of ethyl 2-(4-amino-2-chloropyrimidin-5-yloxy)acetate and ethyl 2-(4-amino-2-bromopyrimidin-5-yloxy)acetate) (3.0 g, 13 mmol), N,N-dimethylformamide (35 mL) and potassium carbonate (0.9 g, 6.5 mmol) was stirred at 60° C. overnight. The mixture was diluted with water (30 mL) and extracted with ethyl acetate (8×50 mL). The organic layers were combined, extracted with water (3×20 mL), saturated aqueous sodium chloride, dried over sodium sulfate, filtered and concentrated. The mixture was separated by preparative HPLC (Column: Kromasil Eternity-5-C₁₈ 30×150 mm; gradient: 5% acetonitrile/water to 20% acetonitrile/water over 12 min, hold 100% acetonitrile 2 min; modifier 0.225% formic acid; wavelength 220 nm) and evaporated to afford 2-chloro-6H-pyrimido[5,4-b][1,4]oxazin-7(8H)-one (60 mg) as a white solid and 2-bromo-6H-pyrimido[5,4-b][1,4]oxazin-7(8H)-one (263 mg) as a white solid.

2-chloro-6H-pyrimido[5,4-b][1,4]oxazin-7(8H)-one: ¹H NMR (400 MHz, DMSO-d₆): δ ppm 4.76 (s, 2H), 8.22 (s, 1H).

2-bromo-6H-pyrimido[5,4-b][1,4]oxazin-7(8H)-one: ¹H NMR (400 MHz, DMSO-d₆): δ ppm 4.75 (s, 2H), 8.17 (s, 1H).

### Preparation 23

### 7-bromo-1H-4,2,1-benzoxathiazine 2,2-dioxide

A solution of 2-amino-4-bromophenol (4.079 g, 21.69 mmol), tetrahydrofuran (50 mL) and chloromethanesulfonyl chloride (2.15 mL, 23.9 mmol) was stirred at room temperature for 30 min. Pyridine (1.93 mL, 23.9 mL) was added and the reaction mixture was stirred at room temperature for 18 h. The mixture was poured into aqueous hydrochloric acid (2 N, 150 mL) and extracted with ethyl acetate (4×50 mL). The combined organic layers were washed with water, dried over magnesium sulfate and filtered through silica gel. The filtrate was concentrated and to the residue obtained was added methanol (80 mL) and potassium carbonate (6 g, 43.4 mmol). The mixture was stirred at reflux for 4 h, at room temperature 48 h and at reflux for 5 h. The reaction mixture was concentrated, quenched with aqueous hydrogen chloride (2 N, 120 mL) and extracted with ethyl acetate (3×55 mL). The combined organic layers were dried over magnesium sulfate, filtered through silica gel, and solvent concentrated. The residue was triturated with diethyl ether/heptanes (˜4:1) and the solid obtained filtered to provide the title compound (1.296 g, 22.6%). ¹H NMR (400 MHz, DMSO-d₆) δ ppm 5.25 (s, 2H), 6.97 (d, J=2.1 Hz, 1H), 7.05 (d, J=8.6 Hz, 1H), 7.14-7.20 (m, 1H), 10.87 (br s, 1H).

### Preparation 24

### 6-chloro-2,2-difluoro-2H-pyrido[3,2-b][1,4]oxazin-3(4H)-one

### Step 1: 2-chloro-N-(6-chloro-3-hydroxypyridin-2-yl)-2,2-difluoroacetamide

A mixture of 2-amino-6-chloro-3-hydroxypyridine (175 mg, 1.21 mmol), triethylamine (340 μL, 2.40 mmol), dichloromethane (12 mL) and 2-chloro-2,2-difluoroacetic anhydride (210 μL, 1.21 mmol) was stirred at 0° C. for 30 min, then at room temperature for 3.5 h. The mixture was concentrated and the residue purified by silica gel chromatography (gradient 0-30% ethyl acetate/heptanes) to provide the title compound as a solid (184 mg, 59%). ¹H NMR (400 MHz, CHLOROFORM-d) δ ppm 7.21 (1H, d, J=8.4 Hz), 7.39 (1H, d, J=8.6 Hz), 8.61 (1H, br s), 8.95 (1H, s).

### Step 2: 6-chloro-2,2-difluoro-2H-pyrido[3,2-b][1,4]oxazin-3(4H)-one

A mixture of 2-chloro-N-(6-chloro-3-hydroxypyridin-2-yl)-2,2-difluoroacetamide (125 mg, 0.486 mmol), t-amyl alcohol (4.8 mL) and potassium t-butoxide in t-butanol (1 N, 1 mL, 1.0 mmol) was stirred at 60° C. overnight. The reaction was cooled to room temperature and concentrated. The residue was dissolved in aqueous hydrochloric acid (1 N, 10 mL) and extracted with ethyl acetate. The organic layer was dried over magnesium sulfate, filtered, and concentrated. The crude residue was purified by silica gel chromatography (gradient 0-30% ethyl acetate/heptanes) to give the title compound (25 mg, 23%) as a solid. ¹H NMR (400 MHz, CHLOROFORM-d) δ ppm 7.12 (1H, d, J=8.4 Hz), 7.48 (1H, d, J=8.4 Hz), 8.29 (1H, br s).

### Preparation 25

### 2-chloro-4-methyl-6H-pyrimido[5,4-b][1,4]oxazin-7(8H)-one

### Step 1: 2,4-dichloro-5-methoxy-6-methylpyrimidine

A mixture of ethyl 2-methoxy-3-oxobutanoate (WO 2006/105222, 500 mg, 3.42 mmol), urea (225 mg, 3.76 mmol), p-toluenesulfonic acid (10 mg) and hexane (20 mL) was refluxed using a Dean-Stark trap for 6 h. The mixture was concentrated and aqueous sodium hydroxide (10%, 10 mL) was added. The mixture was stirred at 95° C. for 30 min. After cooling to room temperature the mixture was acidified with concentrated hydrogen chloride. The mixture was concentrated, dissolved in methanol (16 mL), filtered, and concentrated again to provide 5-methoxy-6-methylpyrimidine-2,4(1H,3H)-dione (400 mg, 75%) as a solid. A mixture of 5-methoxy-6-methylpyrimidine-2,4(1H,3H)-dione (13 g, 83.2 mmol) and phosphoryl chloride (91 mL, 0.98 mol) was stirred at reflux 1 h. The mixture was concentrated. The residue obtained was poured into water and stirred 1 h. The mixture was extracted with ethyl acetate (3×100 mL). The organic layers were combined and concentrated to provide the title compound (8 g, 50%) as a solid. ¹H NMR (400 MHz, CHLOROFORM-d) δ ppm 2.53 (s, 3H), 3.88 (s, 3H).

### Step 2: 2-chloro-5-methoxy-6-methylpyrimidin-4-amine

A mixture of 2,4-dichloro-5-methoxy-6-methylpyrimidine (7 g, 36.3 mmol), dioxane (25 mL) and ammonium hydroxide (28%, 15 mL) was stirred at 100° C. in a sealed reaction vessel. The mixture was cooled to room temperature, diluted with water (20 mL) and extracted with ethyl acetate (5×50 mL). The combined organic layer was concentrated to provide the title compound (3.5 g, 56%) as a solid. ¹H NMR (400 MHz, CHLOROFORM-d) δ ppm 2.19 (s, 3H), 3.61 (s, 3H), 7.26 (br s, 2H).

### Step 3: 2-chloro-4-methyl-6H-pyrimido[5,4-b][1,4]oxazin-7(8H)-one

The title compound was prepared from 2-chloro-5-methoxy-6-methylpyrimidin-4-amine by the general method used for Preparation 22. ¹H NMR (400 MHz, CHLOROFORM-d) δ ppm 2.26 (s, 3H), 4.76 (s, 2H), 11.89 (s, 1H)

### Example 1

### 6-((2R,5R)-2-methyl-5-phenylmorpholino)-2H-pyrido[3,2-b][1,4]oxazin-3(4H)-one

### Method A

A mixture of tris(dibenzylideneacetone)dipalladium(0) (12.8 mg, 0.014 mmol) and 5-(diisopropylphosphino)-1′,3′,5′-triphenyl-1′H-1,4′-bipyrazole (prepared using the method described in Org. Process Res. Dev., 2008, 12(3), 480-489, 13.4 mg, 0.028 mmol) in t-amyl alcohol (0.7 mL) in a sealed reaction vessel was stirred at room temperature under nitrogen for 30 min. (2R,5R)-2-methyl-5-phenylmorpholine (Preparation 2, 100 mg, 0.564 mmol), 6-bromo-2H-pyrido[3,2-b][1,4]oxazin-3(4H)-one (129 mg, 0.564 mmol) and hexamethylphosphoramide (0.516 g, 2.82 mmol) were added to the mixture followed by solid lithium t-butoxide (91.2 mg, 1.13 mmol) and a solution of lithium t-butoxide in t-amyl alcohol (1 M, 2.26 mL, 2.26 mmol). The reaction mixture was stirred at 60° C. overnight. The solution was diluted with ethyl acetate and extracted with saturated aqueous ammonium chloride. The aqueous layer was extracted with ethyl acetate. The combined organic layers were extracted with saturated aqueous sodium chloride, dried over magnesium sulfate, filtered, and concentrated. The crude material was purified by column chromatography on silica gel (gradient: 5-50% ethyl acetate/heptanes). The resulting solid was triturated with acetonitrile to afford the title compound (31 mg, 17%). ¹H NMR (400 MHz, DMSO-d₆) δ ppm 1.12 (3H, d, J=6.0 Hz), 2.81 (1H, m), 3.62 (1H, m), 3.94 (2H, m), 4.26 (1H, m), 4.44 (2H, s), 5.23 (1H, d, J=3.5 Hz), 6.23 (1H, d, J=8.8 Hz), 7.16 (2H, m), 7.28 (4H, m), 10.81 (1H, s).

### Method B

A mixture of tris(dibenzylideneacetone)dipalladium(0) (12.8 mg, 0.014 mmol) and 5-(diisopropylphosphino)-1′,3′,5′-triphenyl-1′H-1,4′-bipyrazole (prepared using the method described in Org. Process Res. Dev., 2008, 12(3), 480-489, 13.4 mg, 0.028 mmol) in t-amyl alcohol (0.7 mL) in a sealed reaction vessel was stirred at room temperature under nitrogen for 30 min. (2R,5R)-2-methyl-5-phenylmorpholine (Preparation 2, 100 mg, 0.564 mmol), and 6-bromo-2H-pyrido[3,2-b][1,4]oxazin-3(4H)-one (129 mg, 0.564 mmol) were added to the mixture followed by dimethylsulfoxide (0.480 mL, 6.77 mmol), solid lithium t-butoxide (91.2 mg, 1.13 mmol) and a solution of lithium t-butoxide in t-amyl alcohol (1 M, 2.26 mL, 2.26 mmol). The reaction mixture was stirred at 60° C. overnight. The solution was diluted with ethyl acetate and extracted with saturated aqueous ammonium chloride. The aqueous layer was extracted with ethyl acetate. The combined organic layers were extracted with saturated aqueous sodium chloride, dried over magnesium sulfate, filtered, and concentrated. The crude material was purified by column chromatography on silica gel (gradient: 5-50% ethyl acetate/heptanes). The resulting solid was triturated with acetonitrile to afford the title compound (72 mg, 39%). ¹H NMR (400 MHz, DMSO-d₆) δ ppm 1.12 (3H, d, J=6.0 Hz), 2.81 (1H, m), 3.62 (1H, m), 3.94 (2H, m), 4.26 (1H, m), 4.44 (2H, s), 5.23 (1H, d, J=3.5 Hz), 6.23 (1H, d, J=8.8 Hz), 7.16 (2H, m), 7.28 (4H, m), 10.81 (1H, s).

### Method C

### Step 1: N-(3-formyl-6-((2R,5R)-2-methyl-5-phenylmorpholino)pyridin-2-yl)pivalamide

A mixture of (2R,5R)-2-methyl-5-phenylmorpholine (Preparation 2, 53 g, 300 mmol), N-(6-chloro-3-formylpyridin-2-yl)pivalamide, N,N-dimethylformamide (150 mL) and diisopropylethylamine (53 mL, 300 mmol) was stirred at 100° C. for 18 h. The mixture was cooled to room temperature and concentrated. The residue was dissolved in ethyl acetate (1 L) and water was added (600 mL). The layers were separated. The organic layer was extracted with aqueous hydrochloric acid (1 N, 500 mL), dried over sodium sulfate, filtered and concentrated. The residue was dissolved in dichloromethane and filtered through silica gel, rinsing through with 50% ethyl acetate in heptanes (3 L) followed by 100% ethyl acetate (500 mL) to provide the title compound. ¹H NMR (400 MHz, CHLOROFORM-d) δ ppm 1.28 (3H, d, J=6.2 Hz), 1.36 (9H, s), 3.04 (1H, dd, J=13.6, 11.0 Hz), 3.75 (1H, m), 4.04 (1H, dd, J=12.0, 3.8 Hz), 4.45 (1H, dd, J=12.1, 1.6 Hz), 6.24 (1H, d, J=9.0 Hz), 7.26 (4H, m), 7.60 (1H, d, J=8.8 Hz), 9.52 (1H, m), 11.58 (1H, br s).

### Step 2: N-(3-hydroxy-6-((2R,5R)-2-methyl-5-phenylmorpholino)pyridin-2-yl)pivalamide

To a 0° C. solution of N-(3-formyl-6-((2R,5R)-2-methyl-5-phenylmorpholino)pyridin-2-yl)pivalamide (88.8 g, 232.9 mmol) in methanol (300 mL) was added urea hydrogen peroxide (87.66 g, 931.9 mmol) and sodium hydroxide in methanol (1 M, 930 mL, 930 mmol). The reaction mixture was stirred at 0° C. for 42 h. Sodium sulfite (100 g) was added and the mixture was stirred at 0° C. for 30 min. The reaction mixture was concentrated. The residue was dissolved in water (1 L) and ethyl acetate (500 mL). The layers were separated and the aqueous layer was extracted with ethyl acetate (2×300 mL). The combined organic layers were extracted with saturated aqueous sodium chloride, dried over magnesium sulfate, filtered and concentrated. The crude product was filtered through silica gel (500 g), eluting with dichloromethane (5 L). The filtrate was concentrated to provide the title compound (42.7 g, 49%). ¹H NMR (400 MHz, CHLOROFORM-d) δ ppm 1.24 (3H, d, J=6.2 Hz), 1.35 (9H, s), 2.93 (1H, m), 3.74 (1H, m), 3.87 (1H, m), 4.05 (1H, m), 4.40 (1H, dd, J=11.7, 1.6 Hz), 5.13 (1H, m), 6.37 (1H, d, J=8.8 Hz), 7.16-7.32 (6H, m), 7.76 (1H, br s), 9.46 (1H, s).

### Step 3: Ethyl 2-(6-((2R,5R)-2-methyl-5-phenylmorpholino)-2-pivalamidopyridin-3-yloxy)acetate

A mixture of N-(3-hydroxy-6-((2R,5R)-2-methyl-5-phenylmorpholino)pyridin-2-yl)pivalamide (61.48 g, 166.4 mmol), sodium iodide (5.0 g, 33.4 mmol), acetone (475 mL), powdered potassium carbonate (34.5 g, 250 mmol) and ethyl bromoacetate (18.4 mL, 166 mmol) was stirred at reflux overnight. The mixture was cooled to room temperature, filtered and concentrated. The residue was dissolved in dichloromethane and filtered through silica gel (400 g) eluting with 10:1 dichloromethane/ethyl acetate and 1:1 dichloromethane/ethyl acetate. The filtrate was concentrated to provide the title compound (58.88 g, 77.7%). ¹H NMR (400 MHz, CHLOROFORM-d) δ ppm 1.24 (6H, m), 1.35 (9H, s), 3.00 (1H, m), 3.75 (1H, m), 4.09 (2H, m), 4.22 (2H, q, J=7.2 Hz), 4.34 (1H, m), 4.51 (2H, s), 5.20 (1H, m), 6.08 (1H, d, J=8.8 Hz), 7.01 (1H, m), 7.21 (3 H, m), 7.42 (2H, m), 8.64 (1H, s).

### Step 4: 6-((2R,5R)-2-methyl-5-phenylmorpholino)-2H-pyrido[3,2-b][1,4]oxazin-3(4H)-one

Aqueous hydrochloric acid (1 N, 560 mL, 560 mmol) and ethyl 2-(6-((2R,5R)-2-methyl-5-phenylmorpholino)-2-pivalamidopyridin-3-yloxy)acetate (51.4 g, 113 mmol) were stirred at reflux 4 h. The reaction mixture was cooled to room temperature, followed by cooling in an ice/water bath. The precipitate was filtered and rinsed with water. The solid was dried in a vacuum oven at 50° C. overnight to give the title compound as a white crystalline solid. (36.4 g, 99%) ¹H NMR (400 MHz, CHLOROFORM-d) δ ppm 1.25 (3H, d, J=6.2 Hz), 2.97 (1H, dd, J=13.1, 10.7 Hz), 3.73 (1H, m), 3.89 (1H, dd, J=13.1, 3.1 Hz), 4.04 (1H, dd, J=11.8, 3.8 Hz), 4.39 (1H, dd, J=11.7, 1.6 Hz), 4.52 (2H, s), 5.16 (1H, m), 6.11 (1H, d, J=8.8 Hz), 7.08 (1H, m), 7.18-7.33 (5H, m), 7.65 (1H, br s).

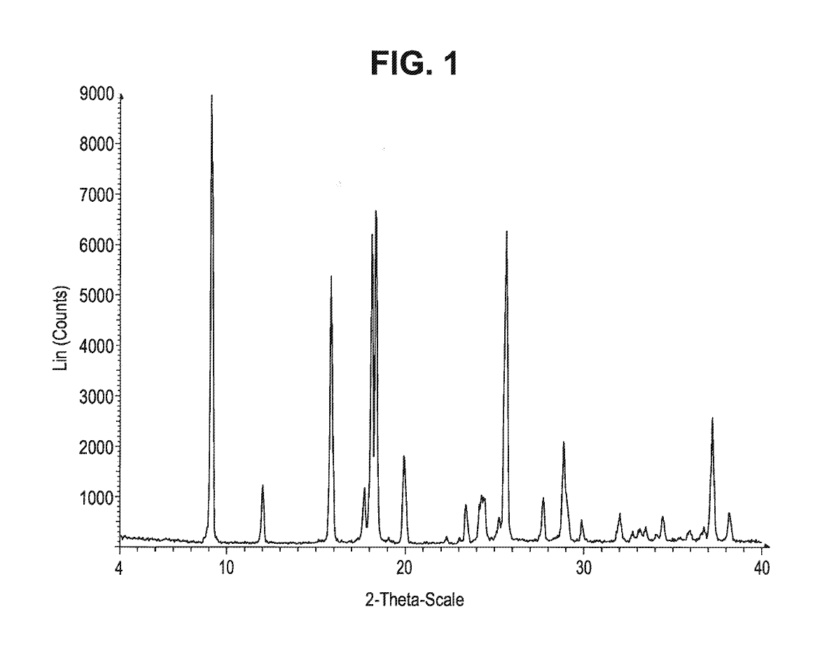

A PXRD of 6-((2R,5R)-2-methyl-5-phenylmorpholino)-2H-pyrido[3,2-b][1,4]oxazin-3(4H)-one is provided in FIG. 1.

### Single Crystal X-Ray Analysis for 6-((2R,5R)-2-methyl-5-phenylmorpholino)-2H-pyrido[3,2-b][1,4]oxazin-3(4H)-one

A crystal suitable for X-ray analysis was prepared by recrystallization from acetonitrile.

Data collection was performed on a Bruker APEX diffractometer at room temperature. Data collection consisted of 3 omega scans at low angle and three at high angle, each with 0.5 step. In addition, 2 phi scans were collected to improve the quality of the absorption correction.

The structure was solved by direct methods using SHELX software suite in the Trigonal space group P3(₁). The structure was subsequently refined by the full-matrix least squares method. All non-hydrogen atoms were found and refined using anisotropic displacement parameters. Locations of all nitrogen and oxygen atoms were confirmed based on reasonable Isotropic/Anisotropic temperature factors and bond angles and distances.

The hydrogen atoms located on nitrogen was found from the Fourier difference map and refined freely. The remaining hydrogen atoms were placed in calculated positions and were allowed to ride on their carrier atoms. The final refinement included isotropic displacement parameters for all hydrogen atoms.

The stereochemistry was determined from the known (R)-2-amino-2-phenylethanol (see Preparation 2) derived chiral center. In addition, the refinement of the opposite enantiomeric Trigonal space group P3(₂) was conducted to compare Flack/Esd parameters, but results were inconclusive due to the absence of a heavy atom(s) in the molecule.

Pertinent crystal, data collection and refinement are summarized in Table 2. Atomic coordinates, bond lengths, bond angles, torsion angles and displacement parameters are listed in Tables 3-6.

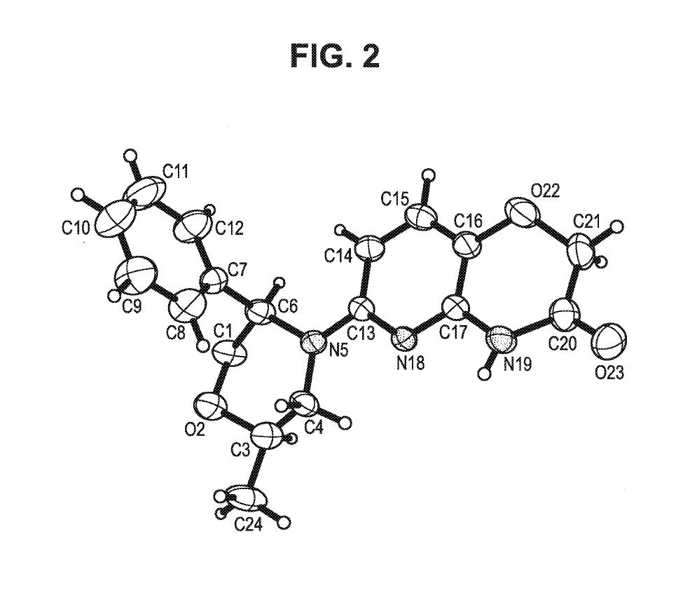

FIG. 2 is an ORTEP Drawing of 6-((2R,5R)-2-methyl-5-phenylmorpholino)-2H-pyrido[3,2-b][1,4]oxazin-3(4H)-one

TABLE 2
Crystal data and structure refinement for 6-((2R,5R)-2-methyl-5-
phenylmorpholino)-2H-pyrido[3,2-b][1,4]oxazin-3(4H)-one.

| | | |
|---|---|---|
| Identification code | I907 | |
| Crystallization | Acetonitrile | |
| Empirical formula | C₁₈H₁₉N₃O₃ | |
| Formula weight | 325.36 | |
| Temperature | 298(2) K | |
| Wavelength | 1.54178 Å | |
| Crystal system | Trigonal | |
| Space group | P3(₁) | |
| Unit cell dimensions | a = 11.1056(2) Å | α = 90°. |
| | b = 11.1056(2) Å | β = 90°. |
| | c = 11.3593(2) Å | γ = 120°. |
| Volume | 1213.29(4) Å³ | |
| Z | 3 | |
| Density (calculated) | 1.336 Mg/m³ | |
| Absorption coefficient | 0.757 mm⁻¹ | |
| F(000) | 516 | |
| Crystal size | 0.26 × 0.20 × 0.04 mm³ | |
| Theta range for data collection | 6.03 to 64.93°. | |
| Index ranges | −9<=h<=13, −13<=k<=10, | |
| | −12<=l<=13 | |
| Reflections collected | 3157 | |
| Independent reflections | 1714 [R(int) = 0.0519] | |
| Completeness to theta = 64.93° | 79.9% | |
| Absorption correction | Empirical | |
| Refinement method | Full-matrix least-squares on F² | |
| Data/restraints/parameters | 1714/1/221 | |
| Goodness-of-fit on F² | 0.781 | |
| Final R indices [I>2sigma(I)] | R1 = 0.0472, wR2 = 0.0965 | |
| R indices (all data) | R1 = 0.0589, wR2 = 0.1009 | |
| Absolute structure parameter | 0.3(4) | |
| Largest diff. peak and hole | 0.189 and − 0.193 e.Å⁻³ | |

TABLE 3
Atomic coordinates (×10⁴) and equivalent isotropic displacement
parameters (Å² × 10³) for 6-((2R,5R)-2-methyl-5-phenylmorpholino)-
2H-pyrido[3,2-b][1,4]oxazin-3(4H)-one. U(eq) is defined as one third
of the trace of the orthogonalized U^{ij} tensor.

| | x | y | z | U(eq) |
|---|---|---|---|---|
| C(1) | 8486(5) | 9230(5) | −5358(4) | 45(1) |
| O(2) | 7248(3) | 8027(3) | −5769(2) | 44(1) |
| C(3) | 6772(5) | 7002(5) | −4854(3) | 41(1) |
| C(4) | 6327(5) | 7472(5) | −3782(3) | 37(1) |
| N(5) | 7420(4) | 8858(4) | −3412(2) | 32(1) |
| C(6) | 8158(5) | 9916(4) | −4318(3) | 34(1) |
| C(7) | 7455(5) | 10733(5) | −4703(3) | 34(1) |
| C(8) | 6055(5) | 10117(6) | −4940(4) | 53(1) |
| C(9) | 5475(6) | 10881(6) | −5370(5) | 62(2) |
| C(10) | 6283(7) | 12254(7) | −5530(5) | 67(2) |
| C(11) | 7675(8) | 12899(6) | −5284(6) | 80(2) |
| C(12) | 8255(6) | 12129(6) | −4865(4) | 59(1) |
| C(13) | 7322(5) | 9285(4) | −2290(3) | 30(1) |
| C(14) | 8210(5) | 10641(5) | −1885(3) | 39(1) |
| C(15) | 8133(5) | 10983(5) | −744(3) | 44(1) |
| C(16) | 7137(5) | 9993(5) | −17(3) | 35(1) |
| C(17) | 6268(4) | 8684(4) | −474(3) | 30(1) |
| N(18) | 6363(4) | 8326(4) | −1569(2) | 31(1) |
| N(19) | 5181(4) | 7700(4) | 221(3) | 38(1) |
| C(20) | 5098(5) | 7879(5) | 1381(3) | 36(1) |
| C(21) | 6334(5) | 9164(6) | 1888(3) | 55(2) |
| O(22) | 6942(4) | 10318(3) | 1121(2) | 55(1) |
| O(23) | 4139(4) | 7067(3) | 2003(2) | 50(1) |
| C(24) | 5560(6) | 5675(5) | −5379(4) | 61(2) |

TABLE 4
Bond lengths [Å] and angles [°] for 6-((2R,5R)-2-
methyl-5-phenylmorpholino)-
2H-pyrido[3,2-b][1,4]oxazin-3(4H)-one.

| | |
|---|---|
| C(1)—O(2) | 1.434(5) |
| C(1)—C(6) | 1.545(5) |
| O(2)—C(3) | 1.434(5) |
| C(3)—C(4) | 1.502(5) |
| C(3)—C(24) | 1.535(6) |
| C(4)—N(5) | 1.467(5) |
| N(5)—C(13) | 1.383(4) |
| N(5)—C(6) | 1.466(5) |
| C(6)—C(7) | 1.528(6) |
| C(7)—C(12) | 1.359(6) |
| C(7)—C(8) | 1.376(7) |
| C(8)—C(9) | 1.385(8) |
| C(9)—C(10) | 1.340(8) |
| C(10)—C(11) | 1.369(9) |
| C(11)—C(12) | 1.387(8) |
| C(13)—N(18) | 1.344(5) |
| C(13)—C(14) | 1.402(6) |
| C(14)—C(15) | 1.366(5) |
| C(15)—C(16) | 1.378(6) |
| C(16)—C(17) | 1.382(5) |
| C(16)—O(22) | 1.388(4) |
| C(17)—N(18) | 1.326(4) |
| C(17)—N(19) | 1.399(5) |
| N(19)—C(20) | 1.342(5) |
| C(20)—O(23) | 1.218(5) |
| C(20)—C(21) | 1.515(6) |
| C(21)—O(22) | 1.412(5) |
| O(2)—C(1)—C(6) | 110.8(3) |
| C(3)—O(2)—C(1) | 107.7(3) |
| O(2)—C(3)—C(4) | 111.5(4) |
| O(2)—C(3)—C(24) | 106.2(3) |
| C(4)—C(3)—C(24) | 111.5(4) |
| N(5)—C(4)—C(3) | 110.7(4) |
| C(13)—N(5)—C(6) | 118.8(3) |
| C(13)—N(5)—C(4) | 117.3(3) |
| C(6)—N(5)—C(4) | 118.5(3) |
| N(5)—C(6)—C(7) | 115.5(3) |
| N(5)—C(6)—C(1) | 108.5(3) |
| C(7)—C(6)—C(1) | 113.0(3) |
| C(12)—C(7)—C(8) | 118.2(5) |
| C(12)—C(7)—C(6) | 118.6(4) |
| C(8)—C(7)—C(6) | 123.1(4) |
| C(7)—C(8)—C(9) | 121.3(5) |
| C(10)—C(9)—C(8) | 119.6(6) |
| C(9)—C(10)—C(11) | 120.3(6) |
| C(10)—C(11)—C(12) | 119.9(6) |
| C(7)—C(12)—C(11) | 120.6(6) |
| N(18)—C(13)—N(5) | 117.2(3) |
| N(18)—C(13)—C(14) | 120.3(3) |
| N(5)—C(13)—C(14) | 122.4(4) |
| C(15)—C(14)—C(13) | 120.1(4) |
| C(14)—C(15)—C(16) | 118.9(4) |
| C(15)—C(16)—C(17) | 118.3(3) |
| C(15)—C(16)—O(22) | 121.2(4) |
| C(17)—C(16)—O(22) | 120.4(4) |
| N(18)—C(17)—C(16) | 123.3(4) |
| N(18)—C(17)—N(19) | 117.6(4) |
| C(16)—C(17)—N(19) | 119.0(3) |
| C(17)—N(18)—C(13) | 119.0(3) |
| C(20)—N(19)—C(17) | 122.4(4) |
| O(23)—C(20)—N(19) | 123.9(4) |
| O(23)—C(20)—C(21) | 121.5(3) |
| N(19)—C(20)—C(21) | 114.6(4) |
| O(22)—C(21)—C(20) | 114.6(3) |
| C(16)—O(22)—C(21) | 113.9(4) |

Symmetry transformations used to generate equivalent atoms.

TABLE 5
Anisotropic displacement parameters (Å² × 10³) for 6-((2R,5R)-2-methyl-
5-phenylmorpholino)-2H-pyrido[3,2-b][1,4]oxazin-3(4H)-one. The
anisotropic displacement factor exponent takes the form:
−2π²[h² a*²U¹¹ + . . . + 2 h k a* b* U¹²]

| | U¹¹ | U²² | U³³ | U²³ | U¹³ | U¹² |
|---|---|---|---|---|---|---|
| C(1) | 36(3) | 42(3) | 46(2) | −3(2) | 13(2) | 12(2) |
| O(2) | 41(2) | 45(2) | 37(1) | −2(1) | 11(1) | 13(2) |
| C(3) | 39(3) | 39(3) | 41(2) | −1(2) | 12(2) | 15(3) |
| C(4) | 39(3) | 33(3) | 31(2) | −5(2) | 2(2) | 11(2) |
| N(5) | 30(2) | 28(2) | 30(1) | 1(1) | 1(1) | 9(2) |
| C(6) | 32(3) | 31(3) | 33(2) | −2(2) | 5(2) | 11(2) |
| C(7) | 34(3) | 33(3) | 32(2) | 5(2) | 8(2) | 15(2) |
| C(8) | 35(3) | 52(4) | 66(3) | 7(2) | 1(2) | 19(3) |
| C(9) | 46(4) | 61(4) | 86(4) | 2(3) | −6(3) | 32(3) |
| C(10) | 71(5) | 56(4) | 84(4) | 13(3) | 5(3) | 41(4) |
| C(11) | 81(5) | 33(4) | 110(4) | 16(3) | −11(4) | 16(3) |
| C(12) | 44(4) | 42(3) | 82(3) | 10(3) | 1(3) | 14(3) |
| C(13) | 30(3) | 29(2) | 28(2) | −2(2) | −3(2) | 12(2) |
| C(14) | 34(3) | 30(3) | 38(2) | 1(2) | −3(2) | 5(2) |
| C(15) | 43(3) | 34(3) | 39(2) | −8(2) | −10(2) | 7(2) |
| C(16) | 41(3) | 37(3) | 29(2) | −3(2) | −3(2) | 21(2) |
| C(17) | 31(2) | 30(3) | 28(2) | −2(2) | −3(2) | 14(2) |
| N(18) | 39(2) | 31(2) | 24(1) | −2(1) | 2(1) | 18(2) |
| N(19) | 38(2) | 36(2) | 33(2) | −2(1) | 6(2) | 14(2) |
| C(20) | 37(3) | 43(3) | 32(2) | 1(2) | 4(2) | 23(2) |
| C(21) | 65(4) | 54(4) | 28(2) | 4(2) | 4(2) | 15(3) |
| O(22) | 68(3) | 42(2) | 35(1) | −10(1) | 5(2) | 14(2) |
| O(23) | 56(2) | 49(2) | 44(2) | 7(2) | 17(2) | 26(2) |
| C(24) | 63(4) | 45(3) | 50(2) | −16(2) | 14(2) | 9(3) |

TABLE 6
Hydrogen coordinates (×10⁴) and isotropic displacement parameters
(Å² × 10³) For 6-((2R,5R)-2-methyl-5-phenylmorpholino)-
2H-pyrido[3,2-b][1,4]oxazin-3(4H)-one.

| | x | y | z | U(eq) |
|---|---|---|---|---|
| H(3) | 7508 | 6827 | −4643 | 80 |
| H(4A) | 5493 | 7495 | −3955 | 80 |
| H(4B) | 6129 | 6822 | −3153 | 80 |
| H(6) | 9037 | 10580 | −3983 | 80 |
| H(8) | 5469 | 9138 | −4804 | 80 |
| H(9) | 4502 | 10429 | −5553 | 80 |
| H(10) | 5882 | 12789 | −5819 | 80 |
| H(11) | 8249 | 13883 | −5401 | 80 |
| H(12) | 9230 | 12585 | −4688 | 80 |
| H(14) | 8872 | 11331 | −2412 | 80 |
| H(15) | 8763 | 11899 | −453 | 80 |
| H(19X) | 4480(70) | 7060(70) | −150(50) | 80 |
| H(21A) | 6045 | 9427 | 2592 | 80 |
| H(21B) | 7030 | 8934 | 2107 | 80 |
| H(24A) | 4802 | 5830 | −5551 | 80 |
| H(24B) | 5261 | 4927 | −4825 | 80 |
| H(24C) | 5860 | 5438 | −6091 | 80 |

### Example 2

### 2-((2R,5R)-2-methyl-5-phenylmorpholino)-6H-pyrimido[5,4-b][1,4]oxazin-7(8H)-one

A mixture of 2-chloro-6H-pyrimido[5,4-b][1,4]oxazin-7(8H)-one (Preparation 22, 100 mg, 0.539 mmol), 1-methyl-2-pyrrolidinone (2 mL), (2R,5R)-2-methyl-5-phenylmorpholine (Preparation 2, 500 mg, 2.8 mmol) and triethylamine (0.3 mL, 2 mmol) was heated to 200° C. under microwave irradiation for 1 h. The reaction was poured into aqueous hydrochloric acid (1 N) and ethyl acetate was added. The layers were separated. The organic layer was extracted with saturated aqueous sodium chloride, dried over sodium sulfate, filtered, and concentrated. The crude material was purified by silica gel column chromatography (gradient: 0-30% heptanes/acetone) to provide the title compound (75 mg, 43%) as a crystalline solid. ¹H NMR (400 MHz, CHLOROFORM-d) δ ppm 1.22 (3H, d, J=6.2 Hz), 2.85 (1H, dd, J=13.7, 10.9 Hz), 3.66 (1H, m), 3.98 (1H, dd, J=11.9, 3.7 Hz), 4.37 (1H, m), 4.46 (1H, dd, J=11.9, 1.2 Hz), 4.57 (2H, s), 5.64 (1H, br s), 7.22 (1H, m), 7.30 (2H, m), 7.41 (2H, m), 7.64 (1H, br s), 7.97 (1H, d, J=1.0 Hz).

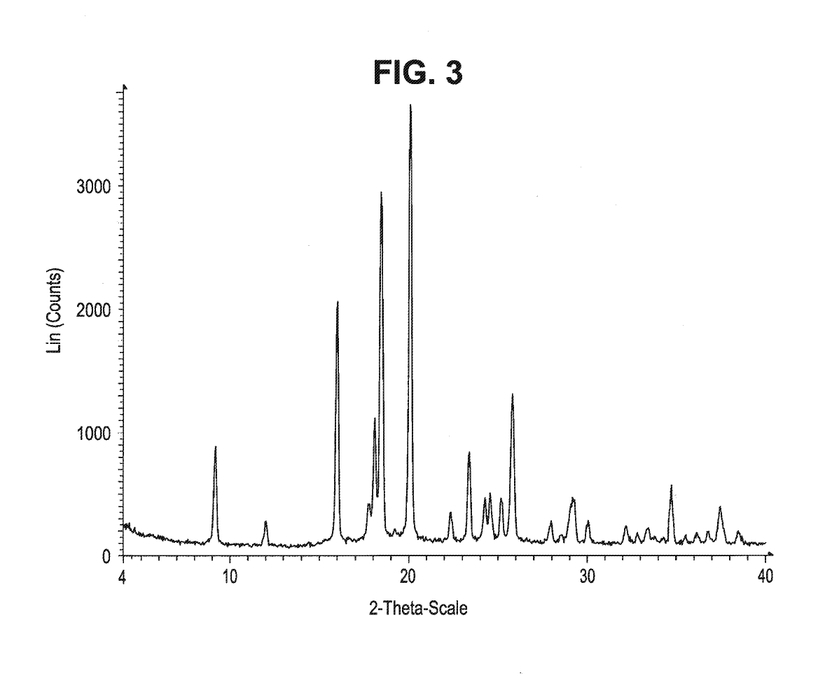

FIG. 3 is a PXRD of 2-((2R,5R)-2-methyl-5-phenylmorpholino)-6H-pyrimido[5,4-b][1,4]oxazin-7(8H)-one.

### Example 3

### 7-((2R,5R)-2-methyl-5-phenylmorpholino)quinoxalin-2(1H)-one

The title compound (109 mg, 30.1%) was prepared from 7-bromoquinoxalin-2(1H)-one and (2R,5R)-2-methyl-5-phenylmorpholine (Preparation 2) by the general method used for Example 45. ¹H NMR (400 MHz, CHLOROFORM-d) δ ppm 1.32 (3H, d, J=6.2 Hz), 3.16 (1H, m), 3.61 (1H, dd, J=12.6, 3.2 Hz), 3.85 (1H, m), 4.12 (1H, m), 4.41 (1H, m), 4.92 (1H, m), 6.51 (1H, dd, J=2.3, 0.4 Hz), 6.90 (1H, m), 7.23 (6H, m), 7.64 (1H, d, J=9.2 Hz), 8.02 (1H, s), 12.00 (1H, m).

### Example 4

### 6-((2R,5R)-2-methyl-5-phenylmorpholino)-2H-benzo[b][1,4]thiazin-3(4H)-one

The title compound (85.1 mg, 52%) was prepared from 6-bromo-2H-1,4-benzothiazin-3(4H)-one and (2R,5R)-2-methyl-5-phenylmorpholine (Preparation 2) by the method used for Example 1, Method B. ¹H NMR (400 MHz, CHLOROFORM-d) δ ppm 1.29 (3H, d, J=6.2 Hz), 3.06 (1H, dd, J=12.3, 10.3 Hz), 3.33 (1H, m), 3.35 (2H, s), 3.82 (1H, m), 4.11 (1H, m), 4.33 (1H, dd, J=11.7, 1.8 Hz), 4.66 (1H, m), 6.18 (1H, d, J=2.7 Hz), 6.53 (1H, dd, J=8.7, 2.6 Hz), 7.11 (1H, d, J=8.8 Hz), 7.25 (8H, m).

### Example 5

### 8-methyl-6-((2R,5R)-2-methyl-5-phenylmorpholino)-2H-pyrido[3,2-b][1,4]oxazin-3(4H)-one

The title compound (12 mg, 4.5%) was prepared from 6-bromo-8-methyl-2H-pyrido[3,2-b][1,4]oxazin-3(4H)-one and (2R,5R)-2-methyl-5-phenylmorpholine (Preparation 2) by the general method used for Example 45. ¹H NMR (500 MHz, CHLOROFORM-d) δ ppm 1.27 (3H, d, J=6.1 Hz), 2.17 (3H, s), 2.96 (1H, dd, J=13.1, 10.6 Hz), 3.75 (1H, m), 3.86 (1H, m), 4.06 (1H, dd, J=11.7, 3.9 Hz), 4.43 (1H, m), 4.56 (2H, s), 5.24 (1H, m), 6.02 (1H, s), 7.24 (1H, m), 7.32 (4H, m), 7.68 (1H, br s).

### Example 6

### 6-[(2R,5R)-2-methyl-5-phenylmorpholin-4-yl]-3,4-dihydro-2H-pyrido[3,2-b][1,4]oxazine

To a 0° C. solution of 6-[(2R,5R)-2-methyl-5-phenylmorpholin-4-yl]-2H-pyrido[3,2-b][1,4]oxazin-3(4H)-one (Example 1, 89 mg, 027 mmol) in tetrahydrofuran (2.8 mL) was added lithium aluminum hydride (2 M in tetrahydrofuran, 280 μL, 0.56 mmol). The solution was stirred at 0° C. until gas evolution ceased then at room temperature for 24 h. Water was added (0.030 mL) followed by aqueous sodium hydroxide (4 N, 30 μL). The mixture was stirred for 15 min and water (60 μL) was added. The reaction mixture was filtered through celite and concentrated. The residue was purified via column chromatography (gradient: 0-50% ethyl acetate in heptanes) to provide the title compound (60 mg, 70%) as a colorless solid. ¹H NMR (400 MHz, CHLOROFORM-d) □ ppm 1.21 (3H, d, J=6.1 Hz), 2.89 (1H, dd, J=12.9, 10.6 Hz), 3.49 (2H, m), 3.71 (1H, m), 3.85 (1H, dd, J=12.9, 3.1 Hz), 4.04 (1H, dd, J=11.6, 3.8 Hz), 4.13 (2H, dd, J=4.7, 4.3 Hz), 4.39 (1H, m), 4.44 (1H, br s), 5.19 (1H, d, J=3.7 Hz), 5.76 (1H, d, J=8.6 Hz), 6.83 (1H, d, J=8.4 Hz), 7.18 (1H, m), 7.26 (1H, m), 7.25 (2H, s), 7.34 (2H, m).

### Example 7

### (±)-6-(cis-5-(2-fluorophenyl)-2-methylmorpholino)-2H-pyrido[3,2-b][1,4]oxazin-3(4H)-one

The title compound (59 mg, 70%) was prepared from 5-(2-fluorophenyl)-2-methylmorpholine (Preparation 6) and 6-bromo-2H-pyrido[3,2-b][1,4]oxazin-3(4H)-one by the general method used for Example 1, Method B. ¹H NMR (400 MHz, CHLOROFORM-d) δ ppm 1.31 (3H, d, J=6.2 Hz), 3.12 (1H, dd, J=13.1, 10.9 Hz), 3.77 (1H, m), 4.07 (2H, m), 4.30 (1H, m), 4.52 (1H, s), 5.27 (1H, m), 6.14 (1H, d, J=8.8 Hz), 7.02 (2H, m), 7.10 (1H, m), 7.21 (1H, m), 7.25 (5H, s).

### Example 8

### 6-((2R,5R)-5-(2-fluorophenyl)-2-methylmorpholino)-2H-pyrido[3,2-b][1,4]oxazin-3(4H)-one

The title compound was prepared by chiral separation of Example 7 by supercritical fluid chromatography on Chiralcel OJ-H column 10×250 mm, mobile phase 85/15 carbon dioxide/methanol, flow rate 10.0 mL/min. UV detection 210 nm. Peak 2: retention time 6.02 min. ¹H NMR (400 MHz, CHLOROFORM-d) δ ppm 1.30 (3H, d, J=6.2 Hz), 3.11 (1H, dd, J=13.1, 10.7 Hz), 3.76 (1H, m), 4.06 (2H, m), 4.30 (1H, m), 4.50 (2H, s), 5.27 (1H, m), 6.12 (1H, d, J=8.8 Hz), 7.03 (3H, m), 7.20 (2H, m), 7.69 (1H, m).

### Example 9

### (±)-6-(cis-5-(4-fluorophenyl)-2-methylmorpholino)-2H-pyrido[3,2-b][1,4]oxazin-3(4H)-one

The title compound (18 mg, 7.5%) was prepared from 6-bromo-2H-pyrido[3,2-b][1,4]oxazin-3(4H)-one and (t)-cis-5-(4-fluorophenyl)-2-methylmorpholine (Preparation 13) by the general method used for Example 45. ¹H NMR (400 MHz, CHLOROFORM-d) δ ppm 1.28 (3H, d, J=6.2 Hz), 2.93 (1H, dd, J=12.8, 10.6 Hz), 3.75 (1H, dt, J=7.4, 3.1 Hz), 3.81 (1H, d, J=12.7 Hz), 4.06 (1H, dd, J=11.7, 3.7 Hz), 4.36 (1H, dd, J=11.8, 1.3 Hz), 4.56 (2H, s), 5.22 (1H, d, J=3.3 Hz), 6.12 (1H, d, J=8.8 Hz), 6.98 (1H, t, J=8.7 Hz), 7.11 (2H, d, J=8.8 Hz), 7.34 (2H, dd, J=8.6, 5.3 Hz), 7.71 (1H, br s).

### Example 10

### 6-((2R,5R)-5-(4-fluorophenyl)-2-methylmorpholino)-2H-pyrido[3,2-b][1,4]oxazin-3(4H)-one

The title compound was prepared by chiral separation of Example 9 by supercritical fluid chromatography on Chiralcel OJ-H column 10×250 mm, mobile phase 70/30 carbon dioxide/methanol, flow rate 10.0 mL/min. UV detection 210 nM. Peak 2: retention time 6.60 min. ¹H NMR (400 MHz, CHLOROFORM-d) δ ppm 1.25 (3H, d, J=6.0 Hz), 2.91 (1H, dd, J=12.9, 10.5 Hz), 3.48 (2H, d, J=5.1 Hz), 3.72 (1H, m), 3.80 (1H, m), 4.03 (1H, dd, J=11.8, 3.8 Hz), 4.34 (1H, dd, J=11.7, 1.4 Hz), 4.53 (2H, s), 5.19 (1H, d, J=3.9 Hz), 6.10 (1H, d, J=8.8 Hz), 6.96 (1H, t, J=8.8 Hz), 7.09 (1H, d, J=8.6 Hz), 7.31 (2H, m).

### Example 11

### (±)-6-(cis-5-(3-fluorophenyl)-2-methylmorpholino)-2H-pyrido[3,2-b][1,4]oxazin-3(4H)-one

The title compound (60 mg, 28%) was prepared from 5-(3-fluorophenyl)-2-methylmorpholine (Preparation 7) and 6-bromo-2H-pyrido[3,2-b][1,4]oxazin-3(4H)-one by the general method used for Example 1, Method B. ¹H NMR (400 MHz, DMSO-d₆) δ ppm 1.13 (3H, d, J=6.1 Hz), 2.79 (1H, m), 3.62 (1H, m), 3.91 (2H, m), 4.26 (1H, m), 4.44 (2H, s), 5.26 (1H, d, J=3.5 Hz), 6.25 (1H, d, J=8.8 Hz), 7.00 (1H, m), 7.14 (3H, m), 7.30 (1H, td, J=8.1, 6.2 Hz), 10.85 (1H, br s).

### Example 12

### (±)-6-(cis-2-methyl-5-phenylmorpholino)-2H-pyrido[3,2-b][1,4]oxazin-3(4H)-one

The title compound (12 mg, 5.5%) was prepared from (±)-cis-2-methyl-5-phenylmorpholine (Preparation 1) and 6-bromo-2H-pyrido[3,2-b][1,4]oxazin-3(4H)-one by the general method used for Example 45. ¹H NMR (400 MHz, CHLOROFORM-d) δ ppm 1.24 (3H, d, J=6.3 Hz), 2.96 (1H, dd, J=13.1, 10.7 Hz), 3.73 (1H, m), 3.89 (1H, dd, J=12.9, 3.1 Hz), 4.04 (1H, dd, J=11.7, 3.9 Hz), 4.38 (1H, dd, J=11.7, 1.6 Hz), 4.52 (2H, s), 5.16 (1H, d, J=4.1 Hz), 6.10 (1H, d, J=8.8 Hz), 7.07 (1H, m), 7.20 (3H, m), 7.29 (3H, m), 7.68 (1H, br s).

### Example 13

### (R)-6-(2,2-dimethyl-5-phenylmorpholino)-2H-pyrido[3,2-b][1,4]oxazin-3(4H)-one

The title compound (9.3 mg, 2.3%) was prepared from (R)-2,2-dimethyl-5-phenylmorpholine (Preparation 8) and 6-bromo-2H-pyrido[3,2-b][1,4]oxazin-3(4H)-one by the general method used for Example 45. ¹H NMR (400 MHz, CHLOROFORM-d) δ ppm 1.26 (6H, d, J=9.4 Hz), 3.26 (1H, d, J=13.7 Hz), 4.00 (3H, m), 4.51 (2H, s), 4.96 (1H, m), 5.98 (1H, d, J=8.8 Hz), 7.01 (1H, d, J=8.8 Hz), 7.30 (4H, m).

### Example 14

### (R)-6-(3-(4-fluorophenyl)morpholino)-2H-pyrido[3,2-b][1,4]oxazin-3(4H)-one

The title compound (50 mg, 36%) was prepared from 3-(4-fluorophenyl)morpholine and 6-bromo-2H-pyrido[3,2-b][1,4]oxazin-3(4H)-one by the general method used for Example 45 followed by chiral separation of the enantiomer mixture by supercritical fluid chromatography on Chiralcel OJ-H column 10×250 mm, mobile phase 70/30 carbon dioxide/ethanol, flow rate 10.0 mL/min. UV detection 210 nM. Peak 1: retention time 4.53 min. ¹H NMR (400 MHz, CHLOROFORM-d) δ ppm 3.47 (1H, m), 3.59 (1H, m), 3.79 (1H, m), 3.99 (2H, m), 4.10 (1H, m), 4.53 (2H, s), 5.01 (1H, m), 6.11 (1H, d, J=8.6 Hz), 6.95 (1H, m), 6.95 (1H, t, J=8.8 Hz), 7.06 (1H, m), 7.32 (2H, m), 7.75 (1H, br s).

### Example 15

### 6-((2S,5R)-2-methyl-5-phenylmorpholino)-2H-pyrido[3,2-b][1,4]oxazin-3(4H)-one

The title compound (47 mg, 13%) was prepared from (2S,5R)-2-methyl-5-phenylmorpholine (Preparation 3) and from 6-bromo-2H-pyrido[3,2-b][1,4]oxazin-3(4H)-one by the general method used for Example 45. ¹H NMR (400 MHz, CHLOROFORM-d) δ ppm 1.26 (3H, d, J=6.2 Hz), 2.79 (1H, dd, J=12.9, 10.1 Hz), 3.54 (1H, dd, J=11.8, 9.9 Hz), 3.98 (2H, m), 4.24 (1H, dd, J=9.9, 4.2 Hz), 4.51 (2H, s), 6.09 (1H, d, J=8.6 Hz), 6.92 (1H, m), 7.19 (1H, m), 7.27 (2H, m), 7.25 (2H, s), 7.59 (1H, br s).

### Example 16

### (R)-6-(3-phenylmorpholino)-2H-pyrido[3,2-b][1,4]oxazin-3(4H)-one

The title compound (125 mg, 66%) was prepared from (R)-3-phenylmorpholine and from 6-bromo-2H-pyrido[3,2-b][1,4]oxazin-3(4H)-one by the general method used for Example 1, Method A. ¹H NMR (400 MHz, DMSO-d₆) δ ppm 3.31 (2H, m), 3.60 (1 H, td, J=11.1, 3.4 Hz), 3.76 (1H, dt, J=13.1, 2.9 Hz), 3.84 (1H, dd, J=11.7, 3.7 Hz), 3.91 (1H, m), 4.14 (1H, m), 4.44 (2H, s), 5.14 (1H, t, J=3.1 Hz), 6.20 (1H, d, J=8.8 Hz), 7.16 (1H, m), 7.28 (4H, m).

### Example 17

### 6-(2-methylmorpholino)-2H-pyrido[3,2-b][1,4]oxazin-3(4H)-one

The title compound (190 mg, 87%) was prepared from 2-methylmorpholine and from 6-bromo-2H-pyrido[3,2-b][1,4]oxazin-3(4H)-one by the general method used for Example 45. ¹H NMR (400 MHz, CHLOROFORM-d) δ ppm 1.24 (3H, d, J=6.2 Hz), 2.49 (1H, dd, J=12.5, 10.3 Hz), 2.84 (1H, m), 3.66 (1H, m), 3.70 (1H, m), 3.78 (1H, dd, J=11.7, 2.1 Hz), 3.87 (1H, m), 3.99 (1H, m), 4.54 (2H, s), 6.22 (1H, d, J=8.8 Hz), 7.14 (1H, m), 7.62 (1H, br s).

### Example 18

### (S)-6-(2-methylmorpholino)-2H-pyrido[3,2-b][1,4]oxazin-3(4H)-one

The title compound was prepared chiral separation of the enantiomer mixture from Example 17 by supercritical fluid chromatography on Chiralcel OJ-H column 10×250 mm, mobile phase 75/25 carbon dioxide/methanol and flow rate 10.0 mL/min. UV detection 210 nM. Peak 2: retention time 7.03 min. ¹H NMR (400 MHz, CHLOROFORM-d) δ ppm 1.24 (3H, d, J=6.3 Hz), 2.49 (1H, m), 2.85 (1H, m), 3.65 (1H, m), 3.70 (1H, m), 3.77 (1H, m), 3.86 (1H, m), 3.98 (1H, m), 4.54 (2H, s), 6.22 (1 H, d, J=8.8 Hz), 7.14 (1H, d, J=8.6 Hz), 7.60 (1H, m).

### Example 19

### (R)-6-(2-methylmorpholino)-2H-pyrido[3,2-b][1,4]oxazin-3(4H)-one

The title compound was prepared chiral separation of the enantiomer mixture from Example 17 by the method described in Example 18. Peak 1: retention time 5.50 min. ¹H NMR (400 MHz, CHLOROFORM-d) δ ppm 1.22 (3H, d, J=6.3 Hz), 2.48 (1H, m), 2.83 (1H, td, J=12.1, 3.5 Hz), 3.64 (2H, m), 3.77 (1H, m), 3.85 (1H, dt, J=12.4, 2.0 Hz), 3.97 (1H, m), 4.53 (2H, s), 6.20 (1H, d, J=8.6 Hz), 7.13 (1H, d, J=8.6 Hz), 8.19 (1H, br s).

### Example 20

### 6-((4aR,9aS)-2,3,9,9a-tetrahydroindeno[2,1-b][1,4]oxazin-4(4aH)-yl)-2H-pyrido[3,2-b][1,4]oxazin-3(4H)-one

The title compound (290 mg, 79%) was prepared from (4aR,9aS)-2,3,4,4a,9,9a-hexahydroindeno[2,1-b][1,4]oxazine (WO 2007/125398) and 6-bromo-2H-pyrido[3,2-b][1,4]oxazin-3(4H)-one by the general method used for Example 1, Method A. ¹H NMR (400 MHz, CHLOROFORM-d) δ ppm 3.01 (2H, m), 3.12 (1H, m), 3.66 (1H, td, J=11.6, 2.4 Hz), 3.78 (1H, m), 3.84 (1H, m), 4.39 (1H, t, J=3.9 Hz), 4.57 (2H, s), 5.52 (1H, m), 6.33 (1H, d, J=8.8 Hz), 6.83 (1H, d, J=7.4 Hz), 7.08 (1H, t, J=7.5 Hz), 7.20 (2H, m), 7.29 (1H, m), 7.58 (1H, br s).

### Example 21

### (R)-6-(3-phenylmorpholino)-2H-benzo[b][1,4]oxazin-3(4H)-one

A mixture of (R)-3-phenylmorpholine (292 mg, 1.8 mmol), tris (dibenzylideneacetone)dipalladium (0) (1.5 mg, 0.018 mmol), 6-bromo-2H-benzo[b][1,4]oxazin-3(4H)-one (342 mg, 1.5 mmol), 2-(2-dicyclohexylphosphanylphenyl)-N,N-dimethylaniline (1.5 mg, 0.36 mmol), lithium bis(trimethylsilyl)amide (1 M solution in hexanes, 3.3 mL) and tetrahydrofuran (6 mL) in was stirred at 70° C. overnight. The reaction mixture was diluted with ethyl acetate and extracted with saturated aqueous ammonium chloride. The aqueous layer was extracted with ethyl acetate. The combined organic layer was extracted with saturated aqueous sodium chloride, dried over magnesium sulfate, filtered, and concentrated. The crude material was purified by silica gel column chromatography (gradient: 0-80% ethyl acetate/heptanes) to afford the title compound (75 mg, 16%) as a light yellow solid. ¹H NMR (400 MHz, CHLOROFORM-d) δ ppm 3.02 (1H, ddd, J=12.1, 9.3, 4.5 Hz), 3.29 (1H, m), 3.55 (1H, dd, J=11.5, 9.0 Hz), 3.93 (3H, m), 4.15 (1H, dd, J=9.0, 3.5 Hz), 4.48 (2H, s), 6.35 (1H, d, J=2.5 Hz), 6.58 (1H, dd, J=8.8, 2.5 Hz), 6.73 (1H, d, J=8.6 Hz), 7.17 (2H, m), 7.25 (3H, m), 7.58 (1H, br s).

### Example 22

### (R)-8-methyl-6-(3-phenylmorpholino)-2H-benzo[b][1,4]oxazin-3(4H)-one

The title compound (10 mg, 8%) was prepared from 3-phenylmorpholine and 6-bromo-2H-pyrido[3,2-b][1,4]oxazin-3(4H)-one by the general method used for Example 21 followed by chiral separation of the racemic product by supercritical fluid chromatography on Chiralcel OJ-H column 10×250 mm, mobile phase 80/20 carbon dioxide/methanol, flow rate 10.0 mL/min. UV detection 210 nM. Peak 1: retention time 3.97 min. ¹H NMR (400 MHz, CHLOROFORM-d) δ ppm 2.07 (3H, s), 2.99 (1H, ddd, J=12.2, 9.2, 4.4 Hz), 3.27 (1H, dt, J=12.2, 2.8 Hz), 3.53 (1H, m), 3.91 (3H, m), 4.14 (1H, dd, J=9.0, 3.5 Hz), 4.49 (2H, s), 6.21 (1H, m), 6.46 (1H, dd, J=2.0, 0.6 Hz), 7.13 (1H, m), 7.20 (2H, m), 7.26 (2H, m), 8.24 (1H, s).

### Example 23

### 6-((2R,4aR,9aS)-2-methyl-2,3,9,9a-tetrahydroindeno[2,1-b][1,4]oxazin-4(4aH)-yl)-2H-pyrido[3,2-b][1,4]oxazin-3(4H)-one

The title compound (30 mg, 20%) was prepared from (4aR,9aS)-2-methyl-2,3,4,4a,9,9a-hexahydroindeno[2,1-b][1,4]oxazine (Preparation 12) and 6-bromo-2H-pyrido[3,2-b][1,4]oxazin-3(4H)-one by the general method used for Example 1, Method B and followed by chiral separation by supercritical fluid chromatography on Chiralcel OJ-H column 10×250 mm, mobile phase 75/25 carbon dioxide/methanol, flow rate 10.0 mL/min. UV detection 210 nm. Peak 2: retention time 9.00 min. ¹H NMR (400 MHz, CHLOROFORM-d) δ ppm 1.12 (3H, d, J=6.2 Hz), 2.58 (1H, m), 2.96 (1H, m), 3.08 (1H, m), 3.66 (1H, m), 3.81 (1H, m), 4.44 (1H, t, J=4.0 Hz), 4.55 (2H, s), 5.43 (1H, d, J=4.1 Hz), 6.31 (1H, d, J=8.8 Hz), 6.83 (1H, d, J=7.4 Hz), 7.06 (1H, t, J=7.4 Hz), 7.19 (2H, m), 7.28 (1H, m), 7.88 (1H, m).

### Example 24

### 6-((2S,4aR,9aS)-2-methyl-2,3,9,9a-tetrahydroindeno[2,1-b][1,4]oxazin-4(4aH)-yl)-2H-pyrido[3,2-b][1,4]oxazin-3(4H)-one

The title compound (29 mg, 20%) was prepared from (4aR,9aS)-2-methyl-2,3,4,4a,9,9a-hexahydroindeno[2,1-b][1,4]oxazine (Preparation 12) and 6-bromo-2H-pyrido[3,2-b][1,4]oxazin-3(4H)-one by the general method used for Example 1, Method B followed by chiral separation by supercritical fluid chromatography on Chiralcel OJ-H column 10×250 mm, mobile phase 75/25 carbon dioxide/methanol, flow rate 10.0 mL/min. UV detection 210 nm. Peak 1: retention time 7.01 min. ¹H NMR (400 MHz, CHLOROFORM-d) δ ppm 1.31 (3H, d, J=6.6 Hz), 2.92 (1H, d, J=16.6 Hz), 3.15 (2H, m), 3.57 (1H, m), 3.94 (1H, m), 4.56 (2H, s), 4.71 (1H, t, J=4.6 Hz), 5.59 (1H, d, J=4.7 Hz), 6.28 (1H, m), 6.91 (1H, d, J=7.4 Hz), 7.10 (1H, t, J=7.4 Hz), 7.21 (2H, m), 7.28 (1H, m), 7.72 (1H, br s).

### Example 25

### 6-(cis-2,6-dimethylmorpholino)-2H-pyrido[3,2-b][1,4]oxazin-3(4H)-one

The title compound (35 mg, 54%) was prepared from cis-2,6-dimethylmorpholine and 6-bromo-2H-pyrido[3,2-b][1,4]oxazin-3(4H)-one by the general method used for Example 1, Method B. ¹H NMR (400 MHz, CHLOROFORM-d) δ ppm 1.24 (6H, d, J=6.2 Hz), 2.43 (2H, m), 3.70 (2H, m), 3.83 (1H, m), 3.86 (1H, m), 4.54 (2H, s.), 6.23 (1H, d, J=8.8 Hz), 7.14 (1H, d, J=8.8 Hz), 7.68 (1H, m).

### Example 26

### 6-(2-ethylmorpholino)-2H-pyrido[3,2-b][1,4]oxazin-3(4H)-one

The title compound (20 mg, 30%) was prepared from 2-ethylmorpholine and 6-bromo-2H-pyrido[3,2-b][1,4]oxazin-3(4H)-one by the general method used for Example 1, Method B. ¹H NMR (400 MHz, CHLOROFORM-d) δ ppm 0.98 (3H, m), 1.58 (2H, m), 2.55 (1H, dd, J=12.5, 10.3 Hz), 2.90 (1H, td, J=12.0, 3.7 Hz), 3.69 (1H, m), 3.77 (2H, m), 3.87 (1H, m), 4.00 (1H, ddd, J=11.5, 3.6, 1.5 Hz), 4.56 (2H, s), 6.30 (1H, d, J=8.8 Hz), 7.17 (1H, m).

### Example 27

### N-(6-((2R,5R)-2-methyl-5-phenylmorpholino)pyridin-2-yl)methanesulfonamide

The title compound (44 mg, 51%) was prepared from (2R,5R)-2-methyl-5-phenylmorpholine (Preparation 2) and N-(6-bromopyridin-2-yl)methanesulfonamide by the general method used for Example 1, Method A. ¹H NMR (400 MHz, CHLOROFORM-d) δ ppm 1.30 (3H, d, J=6.3 Hz), 1.53 (1H, s), 2.82 (3H, s), 3.09 (1H, m), 3.42 (1H, dd, J=12.4, 3.2 Hz), 3.84 (1H, m), 4.31 (1H, dd, J=11.5, 1.8 Hz), 4.72 (1 H, m), 6.18 (1H, s), 6.48 (1H, m), 6.67 (2H, m), 7.17 (1H, m), 7.24 (4H, m).

### Example 28

### 6-((2R,5R)-2-methyl-5-phenylmorpholino)-2H-benzo[b][1,4]oxazin-3(4H)-one

The title compound (123 mg, 33%) was prepared from 6-bromo-2H-benzo[b][1,4]oxazin-3(4H)-one and (2R,5R)-2-methyl-5-phenylmorpholine (Preparation 2) by the general method used for Example 45. ¹H NMR (400 MHz, DMSO-d₆) δ ppm 1.18 (3H, d, J=6.2 Hz), 2.90 (1H, m), 3.72 (1H, m), 3.97 (1H, dd, J=11.6, 3.6 Hz), 4.12 (1H, m), 4.38 (2H, s), 4.67 (1H, m), 6.34 (1H, d, J=2.9 Hz), 6.44 (1H, dd, J=9.0, 2.9 Hz), 6.72 (1H, d, J=9.0 Hz), 7.14 (1H, m), 7.21 (4H, m), 10.37 (1H, s).

### Example 29

### 6-((2R,5R)-5-(3-fluorophenyl)-2-methylmorpholino)-2H-pyrido[3,2-b][1,4]oxazin-3(4H)-one

The title compound was prepared by separating the enantiomer mixture from Example 11 by supercritical fluid chromatography on Chiralcel OJ-H column 10×250 mm, mobile phase 80/20 carbon dioxide/methanol and flow rate 10.0 mL/min. UV detection 210 nm. Peak 1: retention time 5.27 min. ¹H NMR (400 MHz, DMSO-d₆) δ ppm 1.12 (3H, d, J=6.0 Hz), 2.78 (1H, dd, J=13.0, 10.8 Hz), 3.88 (2H, m), 4.25 (1H, d, J=12.1 Hz), 4.43 (2H, s), 5.25 (1H, br s), 6.24 (1H, d, J=8.8 Hz), 6.99 (1H, d, J=2.3 Hz), 7.14 (2H, d, J=8.6 Hz), 7.28 (1H, m), 10.83 (1H, s).

### Example 30

### 2,2-difluoro-6-((2R,5R)-2-methyl-5-phenylmorpholino)-2H-pyrido[3,2-b][1,4]oxazin-3(4H)-one

The title compound (16 mg, 39%) was prepared from 6-chloro-2,2-difluoro-2H-pyrido[3,2-b][1,4]oxazin-3(4H)-one (Preparation 24) and (2R,5R)-2-methyl-5-phenylmorpholine (Preparation 2) by the general method used for Example 1, Method B. ¹H NMR (400 MHz, CHLOROFORM-d) δ ppm 1.25 (3H, d, J=6.2 Hz), 2.98 (1H, dd, J=13.2, 10.8 Hz), 3.72 (1H, m), 3.97 (1H, dd, J=13.0, 3.2 Hz), 4.03 (1H, dd, J=11.9, 3.9 Hz), 4.40 (1H, dd, J=11.9, 1.6 Hz), 5.16 (1H, d, J=4.9 Hz), 6.24 (1H, d, 8.0 Hz), 7.21-7.26 (2H, m), 7.27-7.39 (4H, m), 7.81 (1H, br s).

### Example 31

### 6-((2R,5R)-2-cyclopropyl-5-phenylmorpholino)-2H-pyrido[3,2-b][1,4]oxazin-3(4H)-one

The title compound (21 mg, 35%) was prepared from (2R,5R)-2-cyclopropyl-5-phenylmorpholine (Preparation 14) and 6-bromo-2H-pyrido[3,2-b][1,4]oxazin-3(4H)-one by the general method used for Example 1, Method B. ¹H NMR (400 MHz, CHLOROFORM-d) δ ppm 0.25-0.32 (1H, m), 0.39-0.46 (1H, m), 0.51-0.62 (2H, m), 0.88-0.98 (1H, m), 2.84-2.92 (1H, m), 3.16 (1H, dd, J=13.2, 10.8 Hz), 3.92-4.00 (2H, m), 4.41 (1H, dd, J=11.7, 1.6 Hz), 4.52 (2H, s), 5.14-5.19 (1H, m), 6.12 (1H, d, J=8.8 Hz), 7.07 (1H, dd, J=8.7, 0.7 Hz), 7.18-7.23 (1H, m), 7.24-7.30 (2H, m), 7.31-7.36 (2 H, m), 7.76 (1H, br s).

### Example 32

### (±)-7-(cis-5-(2-fluorophenyl)-2-methylmorpholino)-1H-pyrido[3,4-b][1,4]oxazin-2(3H)-one

The title compound was prepared from (±)-cis-5-(2-fluorophenyl)-2-methylmorpholine (Preparation 6) and 7-chloro-1H-pyrido[3,4-b][1,4]oxazin-2(3H)-one by the general method used for Example 1, Method B. ¹H NMR (400 MHz, CHLOROFORM-d) δ ppm 1.29 (3H, d, J=6.2 Hz), 3.06-3.18 (1H, m), 3.70-3.82 (1H, m), 4.06 (1H, dd, J=11.8, 4.2 Hz), 4.24 (1H, dd, J=13.3, 3.3 Hz), 4.30 (1H, m), 4.53 (2 H, m), 5.15 (1H, d, J=4.5 Hz), 5.90 (1H, s), 6.98-7.08 (2H, m), 7.16-7.25 (2H, m), 7.55 (1H, br s), 7.86 (1H, s).

### Example 33

### 7-((2R,5R)-5-(2-fluorophenyl)-2-methylmorpholino)-1H-pyrido[3,4-b][1,4]oxazin-2(3H)-one

The enantiomer mixture from Example 32 was separated by preparative SFC on Chiralcel OJ-H column 10×250 mm, mobile phase 80/20 carbon dioxide/methanol, flow rate 10.0 mL/min, UV detection at 210 nm, to provide the title compound. Peak 2: retention time 4.54 min. ¹H NMR (400 MHz, CHLOROFORM-d) δ ppm 1.29 (3H, d, J=6.2 Hz), 3.06-3.18 (1H, m), 3.70-3.82 (1H, m), 4.06 (1H, dd, J=11.8, 4.2 Hz), 4.24 (1 H, dd, J=13.3, 3.3 Hz), 4.30 (1H, m), 4.53 (2H, m), 5.15 (1H, d, J=4.5 Hz), 5.90 (1H, s), 6.98-7.08 (2H, m), 7.16-7.25 (2H, m), 7.55 (1H, br s), 7.86 (1H, s).

### Example 34

### (±)-2-(cis-5-(2-fluorophenyl)-2-methylmorpholino)-6H-pyrimido[5,4-b][1,4]oxazin-7(8H)-one

The title compound was prepared from (±)-cis-5-(2-fluorophenyl)-2-methylmorpholine (Preparation 6) and 2-chloro-6H-pyrimido[5,4-b][1,4]oxazin-7(8H)-one (Preparation 22) by the general method used for Example 1, Method B. ¹H NMR (400 MHz, CHLOROFORM-d) δ ppm 1.25 (3H, d, J=6.1 Hz), 3.06 (1H, dd, J=13.6, 10.8 Hz), 3.62-3.72 (1H, m), 3.95-4.01 (1H, m), 4.35-4.40 (1H, m), 4.43 (1H, dd, J=13.5, 3.1 Hz), 4.54 (2H, s), 5.75 (1H, d, J=4.3 Hz), 6.97-7.05 (2H, m), 7.16-7.28 (2H, m), 7.88 (1H, br s), 7.95 (1H, s).

### Example 35

### 2-((2R,5R)-5-(2-fluorophenyl)-2-methylmorpholino)-6H-pyrimido[5,4-b][1,4]oxazin-7(8H)-one

The enantiomer mixture from Example 34 was separated by supercritical fluid chromatography on a Chiralpak AD-H column 10×250 mm, mobile phase 80/20 carbon dioxide/propanol, flow rate 1.0 mL/min, UV detection at 210 nm to provide the title compound. Peak 1, retention time 3.37 min. ¹H NMR (400 MHz, CHLOROFORM-d) δ ppm 1.25 (3H, d, J=6.1 Hz), 3.06 (1H, dd, J=13.6, 10.8 Hz), 3.62-3.72 (1H, m), 3.95-4.01 (1H, m), 4.35-4.40 (1H, m), 4.43 (1H, dd, J=13.5, 3.1 Hz), 4.54 (2H, s), 5.75 (1 H, d, J=4.3 Hz), 6.97-7.05 (2H, m), 7.16-7.28 (2H, m), 7.88 (1H, br s), 7.95 (1H, s).

### Example 36

### 6-(2,3-dihydrospiro[indene-1,3′-morpholin]-4′-yl)-2H-pyrido[3,2-b][1,4]oxazin-3(4H)-one

The title compound was prepared from 2,3-dihydrospiro[indene-1,3′-morpholine] (Preparation 15) and 6-bromo-2H-pyrido[3,2-b][1,4]oxazin-3(4H)-one using the method described in Example 1, Method B. ¹H NMR (400 MHz, CHLOROFORM-d) δ ppm 2.20-2.32 (1H, m), 2.55-2.64 (1H, m), 2.88-3.05 (2H, m), 3.14-3.27 (1H, m), 3.41-3.52 (2H, m), 3.85-3.96 (1H, m), 4.04-4.15 (2H, m), 4.48 (2H, s), 5.65 (1H, d, J=8.8 Hz), 6.73-6.81 (1H, m), 7.06-7.13 (2H, m), 7.17-7.23 (2H, m), 7.54 (1H, br s).

### Example 37

### 7-fluoro-6-((2R,5R)-2-methyl-5-phenylmorpholino)-2H-pyrido[3,2-b][1,4]oxazin-3(4H)-one

A 0° C. mixture of 6-((2R,5R)-2-methyl-5-phenylmorpholino)-2H-pyrido[3,2-b][1,4]oxazin-3(4H)-one (Example 1, 150 mg, 0.46 mmol), N,N-dimethylformamide (2 mL) and 1-chloromethyl-4-fluoro-1,4-diazonabicyclo[2.2.2]octane bis(tetrafluoroborate) (170 mg, 0.47 mmol) was stirred at 0° C. for 30 min, then at room temperature overnight. The reaction mixture was concentrated and the residue partitioned between ethyl acetate and water. The organic layer was washed with saturated aqueous sodium chloride, dried over magnesium sulfate, filtered, and concentrated. The residue was purified by silica gel column chromatography (gradient 0-50% ethyl acetate/heptanes) to provide the title compound (17 mg, 11%) as a solid. ¹H NMR (400 MHz, CHLOROFORM-d) □ ppm 1.23 (3H, d, J=6.3 Hz), 3.00 (1H, dd, J=13.5, 10.4 Hz), 3.55 (1H, m), 3.78-3.88 (1H, m), 4.11 (1H, dd, J=11.9, 3.7 Hz), 4.36 (1H, dd, J=11.8, 1.7 Hz), 4.54 (2H, s), 4.99-5.04 (1H, m), 7.00 (1H, d, J=11.9 Hz), 7.20-7.31 (3H, m), 7.40-7.44 (2H, m), 7.55 (1H, br s).

### Example 38

### 6-(2-methyl-2-phenylmorpholino)-2H-benzo[b][1,4]oxazin-3(4H)-one

The title compound was prepared from 6-bromo-2H-benzo[b][1,4]oxazin-3(4H)-one and 2-methyl-2-phenylmorpholine using the method described in Example 21. ¹H NMR (400 MHz, DMSO-d₆) δ ppm 1.38 (3H, s), 2.79-2.87 (1H, m), 2.90 (1H, d, J=12.5 Hz), 2.94-3.02 (1H, m), 3.28 (2H, s), 3.53-3.62 (1H, m), 3.64-6.71 (1H, m), 3.74-3.84 (1H, m), 4.44 (2H, s), 6.48-6.52 (1H, m), 6.59 (1H, dd, J=8.8, 2.7 Hz), 6.81 (1H, d, J=8.8 Hz), 7.19-7.25 (1H, m), 7.30-7.37 (2H, m), 7.40-7.46 (2H, m), 10.46 (1H, br s).

### Example 39

### 6-((2S,5R)-2-(fluoromethyl)-5-phenylmorpholino)-2H-pyrido[3,2-b][1,4]oxazin-3(4H)-one

The title compound (15 mg, 31%) was prepared from (2S,5R)-2-(fluoromethyl)-5-phenylmorpholine (Preparation 16) and 6-bromo-2H-pyrido[3,2-b][1,4]oxazin-3(4H)-one by the general method used for Example 1, Method B. ¹H NMR (400 MHz, CHLOROFORM-d) δ ppm 3.18 (1H, dd, J=13.1, 11.1 Hz), 3.93 (1H, m), 4.06 (1H, m), 4.44 (2H, m), 4.58 (1H, m), 4.53 (2H, s), 5.18 (1H, m), 5.28 (1H, s), 6.13 (1H, d, J=8.8 Hz), 7.10 (1H, d, J=9.2 Hz), 7.28 (5H, m), 7.77 (1H, br, s).

### Example 40

### 2-((2R,5R)-4-(3-oxo-3,4-dihydro-2H-pyrido[3,2-b][1,4]oxazin-6-yl)-5-phenylmorpholin-2-yl)acetonitrile

### Step 1: 6-((2S,5R)-2-(hydroxymethyl)-5-phenylmorpholino)-2H-pyrido[3,2-b][1,4]oxazin-3(4H)-one

The title compound (230 mg, 67.1%) was prepared from ((2S,5R)-5-phenylmorpholin-2-yl)methanol (Preparation 16, Step 1) and 6-bromo-2H-pyrido[3,2-b][1,4]oxazin-3(4H)-one by the general method used for Example 1, Method B. ¹H NMR (400 MHz, CHLOROFORM-d) δ ppm 3.13 (2H, d, J=3.9 Hz), 3.79 (1H, m), 3.95 (2H, m), 4.15 (1H, m), 4.51 (2H, d, J=2.1 Hz), 4.57 (2H, m), 6.36 (1H, d, J=8.6 Hz), 7.15 (1H, d, J=8.6 Hz), 7.29 (4H, m), 7.47 (2H, m).

### Step 2: 2-((2R,5R)-4-(3-oxo-3,4-dihydro-2H-pyrido[3,2-b][1,4]oxazin-6-yl)-5-phenylmorpholin-2-yl)acetonitrile

A mixture of 6-((2S,5R)-2-(hydroxymethyl)-5-phenylmorpholino)-2H-pyrido[3,2-b][1,4]oxazin-3(4H)-one (57 mg, 0.17 mmol), dichloroethane (5 mL), triethylamine (38.6 μL, 0.273 mmol) and methanesulfonic anhydride (43.1 mg, 1.2 mmol) was stirred at 0° C. for 2 h and at room temperature for 6 h. The reaction mixture was then partitioned between dichloromethane (20 mL) and aqueous sodium hydroxide (1 N, 20 mL). The organic layer was separated, extracted with saturated aqueous sodium chloride, dried over sodium sulfate, filtered and concentrated to afford ((2S,5R)-4-(3-oxo-3,4-dihydro-2H-pyrido[3,2-b][1,4]oxazin-6-yl)-5-phenylmorpholin-2-yl)methyl methanesulfonate (35 mg, 50%). A mixture of ((2S,5R)-4-(3-oxo-3,4-dihydro-2H-pyrido[3,2-b][1,4]oxazin-6-yl)-5-phenylmorpholin-2-yl)methyl methanesulfonate (35 mg, 0.083 mmol), N,N-dimethylformamide (1 mL) and sodium cyanide (82 mg, 1.7 mmol) was stirred at 120° C. for 4 h. The mixture was partitioned between ethyl acetate (10 mL) and aqueous sodium hydroxide (1 N, 10 mL). The aqueous layer was extracted with ethyl acetate (2×50 mL). The combined organic layers were extracted with saturated aqueous sodium chloride (10 mL), dried over sodium sulfate, filtered and concentrated. The residue was purified by silica gel column chromatography (gradient: 0-100% ethyl acetate in heptanes) to provide the title compound (4.5 mg, 15%) as a white solid. ¹H NMR (400 MHz, CHLOROFORM-d) δ ppm 2.63 (2H, m), 3.10 (1H, m), 3.94 (1H, m), 4.10 (2H, m), 4.46 (1H, m), 4.55 (2H, m), 5.16 (1H, br s), 5.27 (2H, m), 6.15 (1H, m), 7.11 (1H, m), 7.30 (4H, m), 7.72 (1H, br s).

### Example 41

### (S)-6-(3-phenylmorpholino)-2H-benzo[b][1,4]oxazin-3(4H)-one

A mixture of 6-bromo-2H-benzo[b][1,4]oxazin-3(4H)-one (197 mg, 0.864 mmol), (S)-3-phenylmorpholine (172.7 mg, 1.058 mmol), 2-(dicylclohexylphosphino)-2′-(N,N-dimethylamino)biphenyl (11.2 mg, 0.028 mmol), tris(dibenzylideneacetone)dipalladium (0) (8.9 mg, 0.01 mmol), tetrahydrofuran (3.3 mL) and lithium bis(trimethylsilyl)amide in tetrahydrofuran (1 M, 1.93 mL, 2 mmol) was stirred at 70° C. overnight. The mixture was cooled to room temperature and saturated aqueous ammonium chloride and ethyl acetate were added. The layers were separated and the aqueous layer was extracted with ethyl acetate (3×). The organic layers were combined, dried over magnesium sulfate, filtered, and concentrated. The residue was purified by silica gel column chromatography (gradient: 0-25% ethyl acetate in heptanes) to afford the title compound (105.6 mg, 39.4%). ¹H NMR (400 MHz, CHLOROFORM-d) δ ppm 3.01(1H, ddd, J=12.2, 9.3, 4.3 Hz), 3.29 (1H, m), 3.54 (1H, dd, J=11.4, 8.9 Hz), 3.92 (3H, m), 4.15 (1H, m), 4.48 (2H, s), 5.28 (1H, s), 6.37 (1H, d, J=2.5 Hz), 6.56 (1H, m), 6.72 (1 H, d, J=8.8 Hz), 7.16 (3H, m), 7.26 (1H, m), 7.24 (2H, s), 7.97 (1H, s).

### Example 42

### 2-((2S,3R,6R)-2,6-dimethyl-3-phenylmorpholino)-6H-pyrimido[5,4-b][1,4]oxazin-7(8H)-one

The title compound (10 mg, 21%) was prepared from (2S,3R,6R)-2,6-dimethyl-3-phenylmorpholine (Preparation 18) and 2-chloro-6H-pyrimido[5,4-b][1,4]oxazin-7(8H)-one (Preparation 22) by the general method used for Example 1, Method B. ¹H NMR (400 MHz, CHLOROFORM-d) δ ppm 1.10 (d, J=6.6 Hz, 3H), 1.40 (d, J=6.2 Hz, 3H), 3.02 (dd, J=13.6, 11.2 Hz, 2H), 3.78-3.89 (m, 1H), 4.07 (qd, J=6.5, 3.4 Hz, 1H), 4.27 (d, J=11.3 Hz, 1H), 4.56 (s, 2H), 7.22-7.33 (m, 3H), 7.66 (d, J=6.6 Hz, 2H), 7.82-7.92 (m, 1H), 7.96 (s, 1H).

### Example 43

### 7-((2S,3R,6R)-2,6-dimethyl-3-phenylmorpholino)-1H-pyrido[3,4-b][1,4]oxazin-2(3H)-one

The title compound (15 mg, 27%) was prepared from (2S,3R,6R)-2,6-dimethyl-3-phenylmorpholine (Preparation 18) and 7-chloro-1H-pyrido[3,4-b][1,4]oxazin-2(3H)-one by the general method used for Example 1, Method B. NMR (400 MHz, CHLOROFORM-d) δ ppm 1.07 (d, J=6.4 Hz, 3H), 1.43 (d, J=6.2 Hz, 3H), 3.04-3.12 (m, 2H), 3.58 (d, J=9.0 Hz, 0H), 3.89-3.96 (m, 1H), 4.16 (qd, J=6.5, 3.4 Hz, 1H), 4.54 (s, 2H), 5.10 (d, J=3.3 Hz, 1H), 5.88 (s, 1H), 7.27 (m, 3H), 7.41 (br s, 1H), 7.48-7.54 (m, 2H), 7.91 (s, 1H).

### Example 44

### 2-((2R,5R)-2-cyclopropyl-5-phenylmorpholino)-6H-pyrimido[5,4-b][1,4]oxazin-7(8H)-one

The title compound (15 mg, 14%) was prepared from (2R,5R)-2-cyclopropyl-5-phenylmorpholine (Preparation 14) and 2-chloro-6H-pyrimido[5,4-b][1,4]oxazin-7(8H)-one (Preparation 22) by the general method used for Example 1, Method B. ¹H NMR (400 MHz, CHLOROFORM-d) δ ppm 0.28-0.48 (m, 2H), 0.52-0.64 (m, 2H), 0.85-0.96 (m, 1H), 2.84 (ddd, J=11.1, 8.2, 2.7 Hz, 1H), 3.07 (dd, J=13.7, 10.9 Hz, 1H), 3.93 (dd, J=11.9, 3.7 Hz, 1H), 4.50 (m, J=11.7, 1.0 Hz, 2H), 4.59 (s, 2H), 5.64 (d, J=3.3 Hz, 1H), 7.27 (s, 1H), 7.29-7.37 (m, 2H), 7.46 (d, J=7.0 Hz, 2H), 7.66 (br s, 1H), 7.99 (s, 1H).

### Example 45

### 5-((2R,5R)-2-methyl-5-phenylmorpholino)benzo[d]oxazol-2(3H)-one

Potassium t-butoxide (88 mg, 0.79 mmol) was added to a solution of 5-bromo-2-benzoxazolinone (28 mg, 0.15 mmol), (2R,5R)-2-methyl-5-phenylmorpholine (Preparation 2, 27 mg, 0.15 mmol), Tris(dibenzylideneacetone) dipalladium(0) (7 mg, 0.008 mmol) and 5-(di-tert-butylphosphino)-1′, 3′,5′-triphenyl-1H-[1,4]bipyrazole (7 mg, 0.015 mmol) in t-amyl alcohol (0.5 mL). The reaction mixture was stirred at 60° C. overnight. The reaction was cooled to room temperature and extracted with ethyl acetate and saturated aqueous ammonium chloride. The organic layer was dried over sodium sulfate and concentrated. The residue was dissolved in dimethylsulfoxide and purified by preparative HPLC Method A. Gradient: 85% water/acetonitrile linear gradient to 100% acetonitrile in 8.5 min. Analytical LCMS Method A: retention time 2.84 minutes; LCMS (ES+): 311.14 (M+H).

### Example 46

### 7-((2R,5R)-2-methyl-5-phenylmorpholino)-1H-pyrido[3,4-b][1,4]oxazin-2(3H)-one

The title compound was prepared from (2R,5R)-2-methyl-5-phenylmorpholine (Preparation 2) and 7-chloro-1H-pyrido[3,4-b][1,4]oxazin-2(3H)-one by the general method used for Example 45. The residue was dissolved in dimethylsulfoxide and purified by preparative HPLC Method A. Gradient: 90% water/acetonitrile linear gradient to 100% acetonitrile in 8.5 min. Analytical LCMS Method A: retention time 1.92 min; LCMS (ES+): 326.17 (M+H).

### Example 47

### 7-[(2R,5R)-2-methyl-5-phenylmorpholin-4-yl]-1H-4,2,1-benzoxathiazine 2,2-dioxide

The title compound was prepared from 7-bromo-1H-4,2,1-benzoxathiazine 2,2-dioxide (Preparation 23) and (2R,5R)-2-methyl-5-phenylmorpholine (Preparation 2) by the general method used for Example 1, Method A. The residue was dissolved in dimethylsulfoxide and purified by preparative HPLC Method A. Gradient: 80% water/acetonitrile linear gradient to 100% acetonitrile in 8.5 min. Analytical LCMS Method A: retention time 3.05 min; LCMS (ES+): 361.11 (M+H).

### Example 48

### (±)-6-(trans-5-(4-fluorophenyl)-2-methylmorpholino)-2H-pyrido[3,2-b][1,4]oxazin-3(4H)-one

The title compound was prepared from 6-bromo-2H-pyrido[3,2-b][1,4]oxazin-3(4H)-one and (±)-trans-5-(4-fluorophenyl)-2-methylmorpholine (Preparation 5) by the general method used for Example 45. The residue was dissolved in dimethylsulfoxide and purified by preparative HPLC Method C. Gradient: 85% water/acetonitrile linear gradient to 100% acetonitrile in 8.5 min. Analytical LCMS Method A: retention time 2.87 min; LCMS (ES+): 344.19 (M+H).

### Example 49

### 6-((2S,5R)-2-(methoxymethyl)-5-phenylmorpholino)-2H-pyrido[3,2-b][1,4]oxazin-3(4H)-one

The title compound was prepared from (2S,5R)-2-(methoxymethyl)-5-phenylmorpholine (Preparation 9) and from 6-bromo-2H-pyrido[3,2-b][1,4]oxazin-3(4H)-one by the general method used for Example 1, Method B. The residue was dissolved in dimethylsulfoxide and purified by preparative HPLC Method B. Gradient: 75% water/acetonitrile linear gradient to 100% acetonitrile in 8.5 min. Analytical LCMS Method A: retention time 2.78 min; LCMS (ES+): 361.11 (M+H).

### Example 50

### (±)-7-(cis-5-(3-fluorophenyl)-2-methylmorpholino)-1H-pyrido[3,4-b][1,4]oxazin-2(3H)-one

The title compound was prepared from (±)-cis-5-(3-fluorophenyl)-2-methylmorpholine (Preparation 7) and 7-chloro-1H-pyrido[3,4-b][1,4]oxazin-2(3H)-one by the general method used for Example 1, Method B. The residue was dissolved in dimethylsulfoxide and purified by preparative HPLC Method B. Gradient: 90% water/acetonitrile linear gradient to 100% acetonitrile in 8.5 min, hold at 100% acetonitrile to 10.0 min. Analytical LCMS Method A: retention time 2.04 min; LCMS (ES+): 344.11 (M+H).

### Example 51

### (±)-2-(cis-5-(3-fluorophenyl)-2-methylmorpholino)-6H-pyrimido[5,4-b][1,4]oxazin-7(8H)-one

The title compound was prepared from (±)-cis-5-(3-fluorophenyl)-2-methylmorpholine (Preparation 7) and 2-chloro-6H-pyrimido[5,4-b][1,4]oxazin-7(8H)-one (Preparation 22) by the general method used for Example 1, Method B. The residue was dissolved in dimethylsulfoxide and purified by preparative HPLC Method B. Gradient: 85% water/acetonitrile linear to 100% acetonitrile in 8.5 min, hold at 100% acetonitrile to 10.0 min. Analytical LCMS Method A: retention time 2.79 min; LCMS (ES+): 345.12 (M+H).

### Example 52

### 6-((2R,5R)-5-(2,4-difluorophenyl)-2-methylmorpholino)-2H-pyrido[3,2-b][1,4]oxazin-3(4H)-one

The title compound was prepared from (2R,5R)-5-(2,4-difluorophenyl)-2-methylmorpholine (Preparation 10) and 6-bromo-2H-pyrido[3,2-b][1,4]oxazin-3(4H)-one by the general method used for Example 1, Method B. The residue was dissolved in dimethylsulfoxide and purified by preparative HPLC Method C. Gradient: 90% water/acetonitrile linear to 100% acetonitrile in 8.5 min, hold 100% acetonitrile to 10.0 min. Analytical LCMS Method A: retention time 3.05 min LCMS (ES+): 362.12 (M+H).

### Example 53

### 6-(2,2-dimethyl-3-phenylmorpholino)-2H-pyrido[3,2-b][1,4]oxazin-3(4H)-one

The title compound was prepared from 2,2-dimethyl-3-phenylmorpholine (Journal of Organic Chemistry (1972), 37 (20), 3130) and 6-bromo-2H-pyrido[3,2-b][1,4]oxazin-3(4H)-one by the general method used for Example 1, Method B. The residue was dissolved in dimethylsulfoxide and purified by preparative HPLC Method C. Gradient: 80% water/acetonitrile linear gradient 100% acetonitrile in 8.5 min, hold at 100% acetonitrile to 10.0 min. Analytical LCMS Method A: retention time 3.03 min; LCMS (ES+): 340.32 (M+H).

### Example 54

### 6-((2R,6R)-2,6-dimethylmorpholino)-2H-pyrido[3,2-b][1,4]oxazin-3(4H)-one

The title compound was prepared from (2R,6R)-2,6-dimethylmorpholine and 6-bromo-2H-pyrido[3,2-b][1,4]oxazin-3(4H)-one by the general method used for Example 1, Method B. The residue was dissolved in dimethylsulfoxide and purified by preparative HPLC Method B. Gradient: 85% water/acetonitrile linear gradient to 100% acetonitrile in 8.5 min, hold at 100% acetonitrile to 10.0 min. Analytical LCMS Method A: retention time 2.34 min; LCMS (ES+): 264.088 (M+H).

### Example 55

### 2-((2R,5R)-5-(4-fluorophenyl)-2-methylmorpholino)-6H-pyrimido[5,4-b][1,4]oxazin-7(8H)-one

The title compound was prepared from (2R,5R)-5-(4-fluorophenyl)-2-methylmorpholine (Preparation 4) and 2-chloro-6H-pyrimido[5,4-b][1,4]oxazin-7(8H)-one (Preparation 22) by the general method used for Example 1, Method B. The residue was dissolved in dimethylsulfoxide and purified by preparative HPLC Method C. Gradient: 80% water/acetonitrile linear gradient to 100% acetonitrile in 8.5 min, hold at 100% acetonitrile to 10.0 min. Analytical LCMS Method A: retention time 2.79 min; LCMS (ES+): 345.12 (M+H).

### Example 56

### (±)-2-(cis-5-(2-methoxyphenyl)-2-methylmorpholino)-6H-pyrimido[5,4-b][1,4]oxazin-7(8H)-one

The title compound was prepared from (±)-cis-5-(2-methoxyphenyl)-2-methylmorpholine (Preparation 11) and 6-bromo-2H-pyrido[3,2-b][1,4]oxazin-3(4H)-one by the general method used for Example 1, Method B. The residue was dissolved in dimethylsulfoxide and purified by preparative HPLC Method A. Gradient: 80% water/acetonitrile linear gradient to 100% acetonitrile in 8.5 min, hold at 100% acetonitrile to 10.0 min. Analytical LCMS Method A: retention time 3.05 min; LCMS (ES+): 356.16 (M+H).

### Example 57

### 6-(cis-2,6-diethylmorpholino)-2H-pyrido[3,2-b][1,4]oxazin-3(4H)-one

The title compound was prepared from cis-2,6-diethylmorpholine (J. Het. Chem. (1984), 21 (3), 647) and 6-bromo-2H-pyrido[3,2-b][1,4]oxazin-3(4H)-one by the general method used for Example 1, Method B. The residue was dissolved in dimethylsulfoxide and purified by preparative HPLC Method C. Gradient: 90% water/acetonitrile linear gradient to 100% acetonitrile in 8.5 min, hold at 100% acetonitrile to 10.0 min. Analytical LCMS Method A: retention time 3.07 min; LCMS (ES+): 292.16 (M+H).

### Example 58

### 7-((2R,5R)-2-methyl-5-phenylmorpholino)-1H-pyrido[2,3-b][1,4]oxazin-2(3H)-one

The title compound was prepared from 7-bromo-1H-pyrido[2,3-b][1,4]oxazin-2-one and (2R,5R)-2-methyl-5-phenylmorpholine (Preparation 2) by the general method used for Example 1, Method B. The residue was dissolved in dimethylsulfoxide and purified by preparative HPLC Method C. Gradient: 95% water/acetonitrile linear gradient to 50% water/acetonitrile in 8.5 min to 100% acetonitrile in 9.0 min, hold at 100% acetonitrile to 10.0 min. Analytical LCMS Method A: retention time 2.32 min; LCMS (ES+): 326.25 (M+H).

### Example 59

### 7-((2R,5R)-2-methyl-5-phenylmorpholino)quinolin-2(1H)-one

The title compound was prepared from 7-bromoquinolin-2(1H)-one and (2R,5R)-2-methyl-5-phenylmorpholine (Preparation 2) by the general method used for Example 45. The residue was dissolved in dimethylsulfoxide and purified by preparative HPLC Method A. Gradient: 80% water/acetonitrile linear gradient to 100% acetonitrile in 8.5 min, hold 100% acetonitrile to 10.0 min. Analytical LCMS Method A: retention time 2.71 min; LCMS (ES+): 321.14 (M+H).

### Example 60

### 6-((2R,5R)-2-methyl-5-phenylmorpholino)-3-oxo-3,4-dihydro-2H-pyrido[3,2-b][1,4]oxazine-7-carbonitrile

### Step 1: 7-bromo-64(2R,5R)-2-methyl-5-phenvlmorpholino)-2H-pyrido[3,2-b][1,4]oxazin-3(4H)-one

A mixture of 6-((2R,5R)-2-methyl-5-phenylmorpholino)-2H-pyrido[3,2-b][1,4]oxazin-3(4H)-one (Example 1, 200 mg, 0.615 mmol), N,N-dimethylformamide (3 mL) and N-bromosuccinimide (110 mg, 0.618 mmol) was stirred in the dark for 1 h. The mixture was concentrated and the residue was purified by silica gel column chromatography (gradient 0-100% ethyl acetate/heptanes) to provide the title compound (217 mg, 87%) as a solid. ¹H NMR (400 MHz, CHLOROFORM-d) δ ppm 1.37 (3H, d, J=6.3 Hz), 3.08-3.11 (2H, m), 3.93-4.04 (2H, m), 4.18 (1H, dd, J=11.7, 4.7 Hz), 4.54 (2H, s), 4.80-4.85 (1H, m), 7.14-7.24 (3H, m), 7.31-7.35 (2H, m), 7.40 (1H, s), 7.54 (1H, br s).

### Step 2: 6-((2R,5R)-2-methyl-5-phenylmorpholino)-3-oxo-3,4-dihydro-2H-pyrido[3,2-b][1,4]oxazine-7-carbonitrile

A mixture of 7-bromo-6-((2R,5R)-2-methyl-5-phenylmorpholino)-2H-pyrido[3,2-b][1,4]oxazin-3(4H)-one (23 mg, 0.057 mmol), zinc cyanide (12 mg, 0.10 mmol), N,N-dimethylformamide (0.5 mL) and tetrakis(triphenylphosphine)palladium(0) (5 mg, 0.004 mmol) was heated to 100° C. under microwave irradiation for 2 h. The reaction mixture was diluted with ethyl acetate and extracted with saturated aqueous sodium bicarbonate. The organic layer was concentrated. The residue was dissolved in dimethylsulfoxide and purified by preparative HPLC Method A. Gradient: 90% water/acetonitrile linear gradient to 100% acetonitrile in 10.5 min, hold at 100% acetonitrile to 12.0 min. Analytical LCMS Method A: retention time 2.96 min; LCMS (ES+): 351.15 (M+H).

### Example 61

### 7-((2R,5R)-5-(4-fluorophenyl)-2-methylmorpholino)-1H-pyrido[3,4-b][1,4]oxazin-2(3H)-one

The title compound was prepared from (2R,5R)-5-(4-fluorophenyl)-2-methylmorpholine (Preparation 4) and 7-chloro-1H-pyrido[3,4-b][1,4]oxazin-2(3H)-one by the general method used for Example 1, Method B. The residue was dissolved in dimethylsulfoxide and purified by preparative HPLC Method B. Gradient: 90% water/acetonitrile linear gradient to 100% acetonitrile in 8.5 min. Analytical LCMS Method A: retention time 2.09 min; LCMS (ES+): 344.12 (M+H).

### Example 62

### (±)-7-(cis-5-(2-methoxyphenyl)-2-methylmorpholino)-1H-pyrido[3,4-b][1,4]oxazin-2(3H)-one

The title compound was prepared from (±)-cis-5-(2-methoxyphenyl)-2-methylmorpholine (Preparation 11) and 7-chloro-1H-pyrido[3,4-b][1,4]oxazin-2(3H)-one by the general method used for Example 1, Method B. The residue was dissolved in dimethylsulfoxide and purified by preparative HPLC Method B. Gradient: 90% water/acetonitrile linear gradient to 100% acetonitrile in 8.5 min. Analytical LCMS Method A: retention time 2.03 min; LCMS (ES+): 356.12 (M+H).

### Example 63

### N-(2-methyl-3-((2R,5R)-2-methyl-5-phenylmorpholino)phenyl)methanesulfonamide

The title compound was prepared from N-(3-bromo-2-methylphenyl)methanesulfonamide (WO 2004/052847) and (2R,5R)-2-methyl-5-phenylmorpholine (Preparation 2) by the general method used for Example 1, Method B. The residue was dissolved in dimethylsulfoxide and purified by preparative HPLC Method B. Gradient: 80% water/acetonitrile linear gradient to 40% water/acetonitrile in 7 min. Analytical LCMS Method A: retention time 3.09 min; LCMS (ES+): 361.16 (M+H).

### Example 64

### (±)-2-(cis-5-(2-chlorophenyl)-2-methylmorpholino)-6H-pyrimido[5,4-b][1,4]oxazin-7(8H)-one

The title compound was prepared from (±)-cis-5-(2-chlorophenyl)-2-methylmorpholine (Preparation 17) and 2-chloro-6H-pyrimido[5,4-b][1,4]oxazin-7(8H)-one (Preparation 22) by the general method used for Example 1, Method B. The residue was dissolved in dimethylsulfoxide and purified by preparative HPLC Method A. Gradient: 85% water/acetonitrile linear gradient to 100% acetonitrile in 8.5 min. Analytical LCMS Method B: retention time 2.98 min; LCMS (ES+): 361.16 (M+H)

### Example 65

### (±)-7-(cis-5-(2-chlorophenyl)-2-methylmorpholino)-1H-pyrido[3,4-b][1,4]oxazin-2(3H)-one

The title compound was prepared from (±)-cis-5-(2-chlorophenyl)-2-methylmorpholine (Preparation 17) and 7-chloro-1H-pyrido[3,4-b][1,4]oxazin-2(3H)-one by the general method used for Example 1, Method B. The residue was dissolved in dimethylsulfoxide and purified by preparative HPLC Method A. Gradient: 90% water/acetonitrile linear gradient to 100% acetonitrile in 8.5 min. Analytical LCMS Method B: retention time 2.16 min; LCMS (ES+): 360.17 (M+H).

### Example 66

### (±)-6-(cis-5-(2-chlorophenyl)-2-methylmorpholino)-2H-pyrido[3,2-b][1,4]oxazin-3(4H)-one

The title compound was prepared from (±)-cis-5-(2-chlorophenyl)-2-methylmorpholine (Preparation 17) and 6-bromo-2H-pyrido[3,2-b][1,4]oxazin-3(4H)-one by the general method used for Example 1, Method B. The residue was dissolved in dimethylsulfoxide and purified by preparative HPLC Method C. Gradient: 85% water/acetonitrile linear gradient to 100% acetonitrile in 8.5 min. Analytical LCMS Method A: retention time 3.0 min; LCMS (ES+): 360.22 (M+H).

### Example 67

### 6-((2R,5R)-5-(2-chlorophenyl)-2-methylmorpholino)-2H-pyrido[3,2-b][1,4]oxazin-3(4H)-one

The enantiomer mixture from Example 66 was separated by supercritical fluid chromatography on a Chiralpak AD-H column 10×250 mm, mobile phase 70/30 carbon dioxide/propanol, flow rate 10.0 mL/min, UV detection at 210 nm to provide the title compound (peak 2, retention time 6.19 min). ¹H NMR (400 MHz, CHLOROFORM-d) δ ppm 1.19 (2H, d, J=6.3 Hz), 1.32 (3H, d, J=6.3 Hz), 3.21 (1H, dd, J=13.1, 10.9 Hz), 3.78 (1H, m), 4.04 (2H, m), 4.26 (1H, dd, J=11.8, 1.7 Hz), 4.49 (2H, s), 5.28 (1H, m), 6.00 (1H, m), 7.04 (1H, m), 7.15 (2H, m), 7.35 (1H, m), 7.69 (1H, br s).

### Example 68

### 4-methyl-2-((2R,5R)-2-methyl-5-phenylmorpholino)-6H-pyrimido[5,4-b][1,4]oxazin-7(8H)-one

The title compound was prepared from (2R,5R)-2-methyl-5-phenylmorpholine (Preparation 2) and 2-chloro-4-methyl-6H-pyrimido[5,4-b][1,4]oxazin-7(8H)-one (Preparation 25) by the general method used for Example 1, Method B. The residue was dissolved in dimethylsulfoxide and purified by preparative HPLC Method C. Gradient: 85% water/acetonitrile linear gradient to 100% acetonitrile in 8.5 min. Analytical LCMS Method A: retention time 2.94 min; LCMS (ES+): 341.27(M+H)

### Example 69

### 6-((2S,3R,6R)-2,6-dimethyl-3-phenylmorpholino)-2H-pyrido[3,2-b][1,4]oxazin-3(4H)-one

The title compound was prepared from (2S,3R,6R)-2,6-dimethyl-3-phenylmorpholine (Preparation 18) and 6-bromo-2H-pyrido[3,2-b][1,4]oxazin-3(4H)-one by the general method used for Example 1, Method B. The residue was dissolved in dimethylsulfoxide and purified by preparative HPLC Method A. Gradient: 80% water/acetonitrile linear gradient to 100% acetonitrile in 8.5 min. Analytical LCMS Method B: retention time 2.87 min; LCMS (ES+): 340.28 (M+H).

### Example 70

### (R)-6-(7-phenyl-2,5-dioxa-8-azaspiro[3.5]nonan-8-yl)-2H-pyrido[3,2-b][1,4]oxazin-3(4H)-one

The title compound was prepared from (R)-7-phenyl-2,5-dioxa-8-azaspiro[3.5]nonane (Preparation 19) and 6-bromo-2H-pyrido[3,2-b][1,4]oxazin-3(4H)-one by the general method used for Example 1, Method B. The residue was dissolved in dimethylsulfoxide and purified by preparative HPLC Method C. Gradient: 85% water/acetonitrile linear gradient to 100% acetonitrile in 8.5 min. Analytical LCMS Method B: retention time 2.46 min; LCMS (ES+): 354.25 (M+H).

### Example 71

### 6-((2R,5R)-2,5-dimethylmorpholino)-2H-pyrido[3,2-b][1,4]oxazin-3(4H)-one

The title compound was prepared from (2R,5R)-2,5-dimethylmorpholine (US 2006/007583) and 6-bromo-2H-pyrido[3,2-b][1,4]oxazin-3(4H)-one by the general method used for Example 1, Method B. The residue was dissolved in dimethylsulfoxide and purified by preparative HPLC Method C. Gradient: 85% water/acetonitrile linear gradient to 100% acetonitrile in 8.5 min. Analytical LCMS Method B: retention time 2.16 min; LCMS (ES+): 264.27 (M+H).

### General Method for Example 72 to 75

To a 0.1 M solution of 6-bromo-2H-benzo[b][1,4]oxazin-3(4H)-one in t-amyl alcohol (125 μmol) were added the amine (125 μmol), potassium hydroxide pellets (88%, 250 μmol), 5-(Di-tert-butylphosphino)-1′,3′,5′-triphenyl-1H-[1,4]bipyrazole (6.25 μmol) and tris(dibenzylideneacetone)dipalladium (0) (3.125 μmol). The mixture was shaken at 100° C. for 16 h, filtered and concentrated.

### Example 72

### 6-(3′,4′-dihydro-2′H-spiro[morpholine-2,1′-naphthalene]-4-yl)-2H-benzo[b][1,4]oxazin-3(4H)-one

The title compound was prepared from 3′,4′-dihydro-2′H-spiro[morpholine-2,1′-naphthalene] (Preparation 20) and purified by preparative HPLC Method D. Gradient: 54% acetonitrile/ammonium hydroxide linear gradient to 84% acetonitrile/ammonium hydroxide in 12 min. Analytical LCMS Method C: retention time 3.406 min; LCMS (ES+): 351 (M+H).

### Example 73

### 6-(spiro[chroman-4,2′-morpholine]-4′-yl)-2H-benzo[b][1,4]oxazin-3(4H)-one

The title compound was purified was prepared from spiro[chroman-4,2′-morpholine] (Preparation 21) and purified by preparative HPLC Method D. Gradient: 51% acetonitrile/ammonium hydroxide linear gradient to 81% acetonitrile/ammonium hydroxide in 12 min. Analytical LCMS Method C: retention time 3.174 min; LCMS (ES+): 353 (M+H).

### Example 74

### 6-(2-methyl-2-p-tolylmorpholino)-2H-benzo[b][1,4]oxazin-3(4H)-one

The title compound was prepared from 2-methyl-2-p-tolylmorpholine and purified by preparative HPLC Method D. Gradient: 51% acetonitrile/ammonium hydroxide linear gradient to 81% acetonitrile/ammonium hydroxide in 12 min. Analytical LCMS Method C: retention time 3.367 min; LCMS (ES+): 339 (M+H).

### Example 75

### 6-(2,3-diphenylmorpholino)-2H-benzo[b][1,4]oxazin-3(4H)-one

The title compound was prepared from 2,3-diphenylmorpholine and purified by preparative HPLC Method D. Gradient: 54% acetonitrile/ammonium hydroxide linear gradient to 84% acetonitrile/ammonium hydroxide in 12 min. Analytical LCMS Method C: retention time 3.067 min; LCMS (ES+): 387 (M+H).

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed:

1. A compound of the Formula I 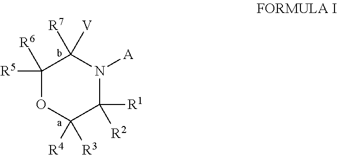
a prodrug thereof, or a pharmaceutically acceptable salt of said compound or of said prodrug;
wherein R¹ and R², are each independently H, (C₁-C₄)alkyl or cyclo(C₃-C₆)alkyl said (C₁-C₄)alkyl optionally mono-substituted with (C₁-C₄)alkoxy or cyano or optionally substituted with one to nine fluoros and said cyclo(C₃-C₆)alkyl optionally substituted with one to six fluoros;
wherein R³, R⁴, R⁵ and R⁶ are each independently H, phenyl, (C₁-C₄)alkyl, cyclo(C₃-C₆)alkyl, or cyclooxa(C₃-C₆)alkyl, said (C₁-C₄)alkyl optionally mono-substituted with (C₁-C₄)alkoxy or cyano or optionally substituted with one to nine fluoros and said cyclo(C₃-C₆)alkyl optionally substituted with one to six fluoros;
wherein at least three of R¹, R², R³, R⁴, R⁵ and R⁶ are H;
or wherein R³ and R⁴ can be linked together to form a three to six membered ring optionally having one oxygen, said ring optionally fused to phenyl;
wherein V is H, phenyl, naphthyl, (C₁-C₄)alkyl or cyclo(C₃-C₆)alkyl, said phenyl, naphthyl, (C₁-C₄)alkyl or cyclo(C₃-C₆)alkyl may optionally be mono-, di- or tri-substituted with R⁸ with the proviso that if V is H, then at least two of R¹, R², R³, R⁴, R⁵, R⁶ or R⁷ are not H;
R⁷ is H or wherein when V is phenyl V is optionally linked together with R⁷ to form a fused nine to ten membered carbobicyclic ring;
or wherein when V is phenyl it is optionally linked together with R⁵ to form the tricyclic moiety 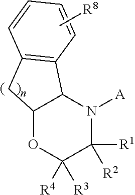
R⁸ is H, halo, (C₁-C₄)alkyl, cyclo(C₃-C₆)alkyl or (C₁-C₄)alkoxy, said (C₁-C₄)alkyl optionally substituted with from one to nine fluoros;
n is 1 or 2;
wherein A is 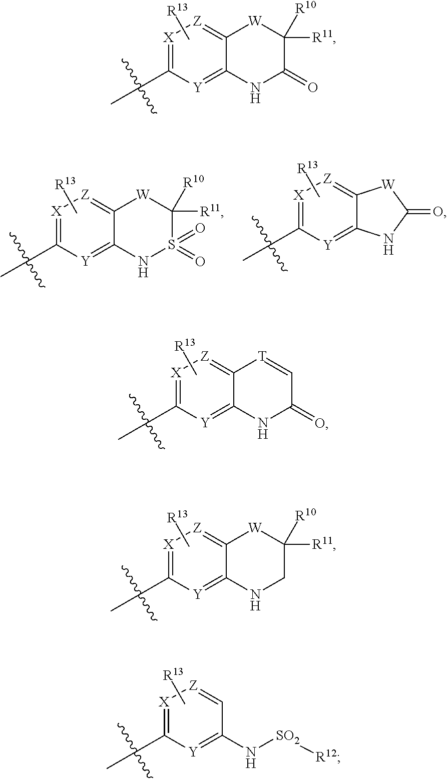
T is CH or N;
X, Y and Z are independently CH or N;
W is CH₂, O, S or NH;
R¹⁰ and R¹¹ are independently H or fluoro;
R¹² is (C₁-C₄)alkyl or cyclo(C₃-C₆)alkyl said (C₁-C₄)alkyl or cyclo(C₃-C₆)alkyl optionally substituted with one to nine fluoros; and
R¹³ is H, (C₁-C₄)alkyl, halo or cyano.

2. A compound as recited in claim 1 wherein
the morpholine C^{a} is (R);
the morpholine C^{b} is (R); and
A is

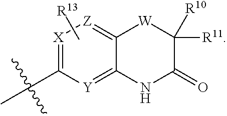

3. A compound as recited in claim 2 wherein
V is phenyl;
W is O;
X is CH;
Y is N;
Z is CH;
R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

4. A compound as recited in claim 3 wherein
R³ is (C₁-C₄)alkyl or cyclo(C₃-C₆)alkyl;
R⁸ is H, halo, (C₁-C₄)alkyl, cyclo(C₃-C₆)alkyl or (C₁-C₄)alkoxy;
R¹⁰ and R¹¹ are H; and
R¹³ is H.

5. A compound as recited in claim 4 wherein
R³ is (C₁-C₄)alkyl; and
R⁸ is H, halo or (C₁-C₄)alkyl.

6. A compound as recited in claim 1 wherein
the morpholine C^{a} is (R);
the morpholine C^{b} is (R);
A is 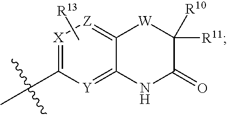
V is phenyl;
W is O;
X is CH;
Y is CH;
Z is CH;
R¹, R², R⁴, R⁵ and R⁸ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

7. A compound as recited in claim 1 wherein
the morpholine C^{a} is (R);
the morpholine C^{b} is (R);
A is 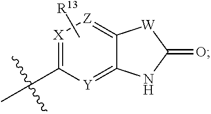
V is phenyl;
W is O;
R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

8. A compound as recited in claim 1 wherein
the morpholine C^{a} is (R);
the morpholine C^{b} is (R);
A is 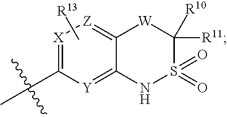
V is phenyl;
W is O;
R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

9. A compound as recited in claim 1 wherein
the morpholine C^{a} is (R);
the morpholine C^{b} is (R);
A is 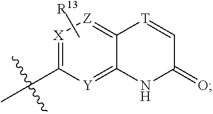
V is phenyl;
R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

10. A compound as recited in claim 1 wherein
the morpholine C^{a} is (R);
the morpholine C^{b} is (R);
A is 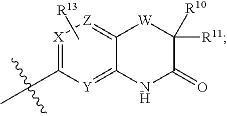
V is phenyl;
W is O;
X is N;
Y is CH;
Z is CH;
R¹, R², R⁴, R⁵ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

11. A compound as recited in claim 1 wherein
the morpholine C^{a} is (R);
the morpholine C^{b} is (R);
A is 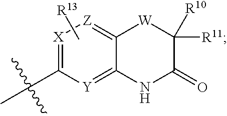
V is phenyl;
W is O;
X is N;
Y is N;
Z is CH;
R¹, R², R⁴, R⁶ and R⁶ are each H;
R³ is H, (C₁-C₄)alkyl or cyclo(C₃-C₆)alkyl, said (C₁-C₄)alkyl optionally substituted with one to nine fluoros and said cyclo(C₃-C₆)alkyl optionally substituted with one to six fluoros;
R⁸ is H, halo, (C₁-C₄)alkyl, cyclo(C₃-C₆)alkyl or (C₁-C₄)alkoxy, said (C₁-C₄)alkyl optionally substituted with from one to nine fluoros; and
R¹³ is H or (C₁-C₄)alkyl.

12. A compound as recited in claim 1 wherein the compound is selected from
6-((2R,5R)-2-methyl-5-phenylmorpholino)-2H-pyrido[3,2-b][1,4]oxazin-3(4H)-one,
6-((2R,5R)-5-(4-fluorophenyl)-2-methylmorpholino)-2H-pyrido[3,2-b][1,4]oxazin-3(4H)-one,
(R)-6-(2,2-dimethyl-5-phenylmorpholino)-2H-pyrido[3,2-b][1,4]oxazin-3(4H)-one,
2-((2R,5R)-2-methyl-5-phenylmorpholino)-6H-pyrimido[5,4-b][1,4]oxazin-7(8H)-one,
6-((2R,5R)-2-methyl-5-phenylmorpholino)-2H-benzo[b][1,4]oxazin-3(4H)-one,
7-((2R,5R)-2-methyl-5-phenylmorpholino)-1H-pyrido[3,4-b][1,4]oxazin-2(3H)-one,
2-((2R,5R)-4-(3-oxo-3,4-dihydro-2H-pyrido[3,2-b][1,4]oxazin-6-yl)-5-phenylmorpholin-2-yl)acetonitrile,
6-((2R,5R)-5-(2-fluorophenyl)-2-methylmorpholino)-2H-pyrido[3,2-b][1,4]oxazin-3(4H)-one,
6-(cis-2,6-dimethylmorpholino)-2H-pyrido[3,2-b][1,4]oxazin-3(4H)-one,
6-((2R,5R)-5-(3-fluorophenyl)-2-methylmorpholino)-2H-pyrido[3,2-b][1,4]oxazin-3(4H)-one,
2-((2R,5R)-5-(2-fluorophenyl)-2-methylmorpholino)-6H-pyrimido[5,4-b][1,4]oxazin-7(8H)-one,
7-((2R,5R)-5-(2-fluorophenyl)-2-methylmorpholino)-1H-pyrido[3,4-b][1,4]oxazin-2(3H)-one,
6-((2R,5R)-5-(2,4-difluorophenyl)-2-methylmorpholino)-2H-pyrido[3,2-b][1,4]oxazin-3(4H)-one,
6-((2S,5R)-2-(fluoromethyl)-5-phenylmorpholino)-2H-pyrido[3,2-b][1,4]oxazin-3(4H)-one,
6-((2S,3R,6R)-2,6-dimethyl-3-phenylmorpholino)-2H-pyrido[3,2-b][1,4]oxazin-3(4H)-one and
6-((2R,5R)-5-(2-chlorophenyl)-2-methylmorpholino)-2H-pyrido[3,2-b][1,4]oxazin-3(4H)-one.

13. 6-(2-methyl-5-phenylmorpholino)-2H-pyrido[3,2-b][1,4]oxazin-3(4H)-one.

14. 6-((2R,5R)-2-methyl-5-phenylmorpholino)-2H-pyrido[3,2-b][1,4]oxazin-3(4H)-one or a pharmaceutically acceptable salt thereof.

15. A compound having the Formula II

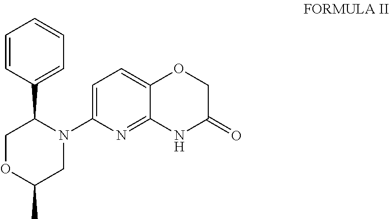

16. A method for treating cardiovascular conditions, renal conditions, liver conditions, inflammatory conditions, pain, retinopathy, neuropathy, insulinopathy, diabetic nephropathy, edema, endothelial dysfunction or baroreceptor dysfunction in a mammal (including a human being either male or female) by administering to a mammal in need of such treatment a cardiovascular conditions, renal conditions, liver conditions, inflammatory conditions, pain, retinopathy, neuropathy, insulinopathy, diabetic nephropathy, edema, endothelial dysfunction or baroreceptor dysfunction treating amount of a compound of claim 1, a prodrug thereof, or a pharmaceutically acceptable salt of said compound or of said prodrug.

17. A method as recited in claim 16 wherein diabetic nephropathy is treated.

18. A pharmaceutical composition which comprises a therapeutically effective amount of a compound of claim 1, a prodrug thereof, or a pharmaceutically acceptable salt of said compound or of said prodrug and a pharmaceutically acceptable carrier, vehicle or diluent.

19. A pharmaceutical combination composition comprising: a therapeutically effective amount of a composition comprising
a first compound, said first compound being a compound of claim 1, a prodrug thereof, or a pharmaceutically acceptable salt of said compound or of said prodrug;
a second compound, said second compound being a diuretic; and
a pharmaceutical carrier, vehicle or diluent.

20. A pharmaceutical combination composition as recited in claim 19 wherein the second compound is a torsemide.
